(19) United States
(12) Patent Application Publication
(10) Pub. No.: US 2025/0288431 A1
(43) Pub. Date: Sep. 18, 2025

(54) INSTRUMENTATION FOR TOTAL SPINAL JOINT REPLACEMENT

(71) Applicant: 3Spine, Inc., Chattanooga, TN (US)

(72) Inventors: Christian Davis, Cambridge, MA (US); Marc M. Peterman, Duxbury, MA (US); Steven C. Humphreys, Soldotna, AK (US); Scott Hodges, Chattanooga, TN (US); Ron Yarbrough, Round Rock, TX (US)

(21) Appl. No.: 19/076,602

(22) Filed: Mar. 11, 2025

Related U.S. Application Data
(63) Continuation of application No. PCT/US23/32543, filed on Sep. 12, 2023.
(60) Provisional application No. 63/375,379, filed on Sep. 12, 2022.

(51) Int. Cl. A61F 2/46; A61B 17/00; A61B 17/16; A61B 17/17

(52) U.S. Cl. CPC A61F 2/4611; A61B 17/1671; A61B 17/1757; A61B 2017/00464

(57) ABSTRACT

The inventions disclosed herein relate to methods, devices and systems of surgical instruments for use in spinal surgery. More specifically, the inventions relate to methods, devices, and systems of surgical instruments for use in spinal surgery to restore spinal alignment and motion.

### CROSS-REFERENCE TO RELATED APPLICATIONS

This application in a continuation application of Patent Cooperation Treaty Application Serial No. PCT/US23/32543 entitled “INSTRUMENTATION FOR TOTAL SPINAL JOINT REPLACEMENT” filed Sep. 12, 2023, which claims the benefit of U.S. Provisional Patent Application Ser. No. 63/375,379 entitled “SURGICAL INSTRUMENTATION FOR TOTAL SPINAL JOINT REPLACEMENT” filed Sep. 12, 2022, the disclosures of which are incorporated by reference herein in their entireties.

### TECHNICAL FIELD

The invention relates to methods, devices, and systems for spine surgical procedures. More specifically, the invention relates to methods, devices and systems of surgical instruments used to restore spinal alignment and motion.

### BACKGROUND OF THE INVENTION

Spinal disorders such as degenerative disc disease, disc herniation, osteoporosis, spondylolisthesis, stenosis, scoliosis and other curvature abnormalities may result from a variety of factors including trauma, disease and degenerative conditions caused by injury and/or aging. Spinal disorders typically result in symptoms including pain, nerve damage, and partial or complete loss of mobility.

While non-surgical treatments such as medication, rehabilitation and exercise can be effective in addressing many spinal pathologies, such no or less-invasive treatments may fail to adequately address and/or relieve symptoms experienced by some patient populations. In such cases, surgical treatment of a spinal disorder may be necessitated, including surgical corrections, fusion, fixation, discectomy, laminectomy and the placement of implantable prosthetics. As part of a surgical treatment regimen, spinal implants are often used to provide stability to a treated region, as well as redirect stresses away from a damaged or defective region of the spine and restore proper alignment. Surgical instruments are employed, to successfully deploy the spinal implants into their targeted region. This disclosure describes various improvements existing technologies in the art.

### BRIEF DESCRIPTION OF THE SEVERAL VIEWS OF THE DRAWINGS

FIGS. 1A-1E depicts different plan views of one embodiment of a powered reciprocating system;

FIG. 2 depicts a top plan view of one embodiment of the surgical instrumentation kit for a total joint replacement spinal implant system procedure;

FIGS. 3A-3H depicts different plan views of one embodiment of closed surgical instrument kit;

FIG. 4 depicts exploded isometric view of one embodiment of a case tray lid and base;

FIGS. 5A-5F depicts different plan views of one embodiment of a base of a case tray of FIG. 4.

FIGS. 6A-6E depicts an isometric view of one embodiment of a lid for the case tray of FIG. 4;

FIGS. 7A-7F depicts different plan views of one embodiment of a manual handle;

FIGS. 8A-8C depicts different plan views of one embodiment of an AO connector;

FIGS. 9A-9F depicts different plan views and a cross-sectional view of one embodiment of a distractor tool;

FIGS. 9G-9H depicts a front and side view of the various available sizes of the distractor tool of FIGS. 9A-9F;

FIGS. 10A-10F depicts different plan views and across-sectional view of embodiment of a height trial tool;

FIGS. 10G-10H depicts a front and side view of the various available sizes of the height trial tool of FIGS. 10A-10F;

FIGS. 11A-11G depicts different plan views and across-sectional view of one embodiment of a length trial tool;

FIGS. 11H-11I depicts a front and side view of the various available sizes of the length trial tool of FIGS. 11A-11G;

FIGS. 11J-11K depicts a side view of an assembled length trial tool and the length trial tool inserted into an intervertebral disc space;

FIGS. 12A-12H depicts different plan views and across-sectional view of one embodiment of a flat rasp;

FIGS. 12I-12K depicts different plan views of a head of a flat rasp of FIGS. 12A-12H;

FIGS. 12L-12M depicts a front view of the various available sizes of the flat rasp of 12A-12H;

FIGS. 13A-13H depicts different plan views and across-sectional view of one embodiment of a keel rasp;

FIGS. 13I-13K depicts different plan views of a head of a keel rasp of FIGS. 13A-13H;

FIGS. 13L-13M depicts a front view of the various available sizes of the keel rasp of 13A-13H;

FIGS. 14A-14G depicts different plan views of one embodiment of an alignment guide assembly;

FIGS. 15A-15BI depicts cross-sectional views of the alignment guide assembly of FIGS. 14A-14G;

FIGS. 15C-15D depicts an isometric exploded view of the alignment guide assembly of FIGS. 14A-14G;

FIGS. 16A-16H depicts different plan views and across-sectional view of one embodiment of a guide arm;

FIGS. 17A-17H depicts different plan views and across-sectional view of one embodiment of a guide base;

FIGS. 18A-18H depicts different plan views and across-sectional view of one embodiment of a guide latch;

FIGS. 19A-19G depicts different plan views and across-sectional view of one embodiment of a guide button;

FIGS. 20A-20E depicts different side and isometric views of an assembly one an alignment guide system;

FIGS. 20F-20G depicts magnified side views of a first end of an alignment guide system;

FIGS. 21A-21D depicts side and isometric views of a method of use of an alignment guide system;

FIGS. 22A-22G depicts different views of one embodiment of an inserter assembly;

FIGS. 22H-22J depicts cross-sectional and transparent views of the inserter assembly of FIGS. 22A-22G;

FIG. 23 depicts an exploded isometric view of the inserter assembly of FIGS. 22A-22G;

FIGS. 24A-24G depicts different plan views and a cross-sectional view of one embodiment of a handle;

FIGS. 25A-25F depicts different plan views and a cross-sectional view of one embodiment of a handle core;

FIGS. 26A-26E depicts different plan views of one embodiment of an end cap;

FIGS. 27A-27G depicts different plan views and a cross-sectional view of one embodiment of a locking knob;

FIGS. 28A-28E depicts different plan views and a cross-sectional view of one embodiment of an inserter shaft;

FIGS. 28F-28J depicts different magnified views of the inserter shaft of FIGS. 28A-28E;

FIGS. 29A-29J depicts different plan views and a magnified view of one embodiment of a grasping tip;

FIGS. 30A-30F depicts different plan views and a cross-sectional view of one embodiment of a draw bar;

FIGS. 30G-30J depicts magnified views of the draw bar of FIGS. 30A-30F;

FIGS. 31A-31J depicts different plan views and across-sectional view of one embodiment of a screw guide;

FIGS. 32A-32H depicts different plan views and a cross-sectional view of one embodiment of a screwdriver;

FIGS. 33A-33D depicts different views of one embodiment of a preparation or assembly of an inserter assembly and a spinal implant;

FIGS. 34A-34E depicts cross-sectional views of the preparation or assembly of the inserter assembly and the spinal implant of FIGS. 33A-33D;

FIGS. 35A-35F depicts different views of one embodiment of a method of use for the inserter assembly and screw guide within an intervertebral space to deploy the spinal implant;

FIGS. 36A-36H depicts different plan views and a cross-sectional view of one embodiment of a removal driver;

FIGS. 37A-37F depicts different plan views and a cross-sectional view of one embodiment of an implant removal hook tool;

FIGS. 37G-37I depicts different magnified views of the removal hook tool of FIGS. 37A-37F;

FIGS. 38A-38H depicts different plan views and a cross-sectional view of one embodiment of a slap hammer assembly;

FIG. 39 depicts an isometric exploded view of the slap hammer assembly of FIGS. 38A-38H;

FIGS. 40A-40F depicts different plan views and a cross-sectional view of one embodiment of a stop of the slap hammer assembly;

FIGS. 41A-41D depicts different plan views of one embodiment of a hammer body or weight of the slap hammer assembly;

FIGS. 42A-42F depicts different plan views and a cross-sectional view of one embodiment of a guide shaft of the slap hammer assembly;

FIGS. 42G-42J depicts different magnified views of the guide shaft of the slap hammer assembly;

FIGS. 43A-43B depicts a cross sectional views of one embodiment of a retention mechanism;

FIGS. 44A-44B depicts a side view of one embodiment of a method of use for the removal driver;

FIGS. 45A-45C depicts a side view and isometric view of a method of use for the removal hook tool;

FIGS. 46A through 46F depict various views of an embodiment of a protection sleeve for a powered rasping tool; and

FIGS. 47A through 47D depict steps of assembling the sleeve of FIGS. 46A through 46F onto a powered handpiece and rasp.

### DETAILED DESCRIPTION OF THE INVENTION

Various features of the present invention include the realization of a need for improved surgical implants, tools, instrumentation and/or procedures for addressing and/or treating spinal pathologies. It is one objective of this invention to provide improved surgical instrumentation for preparing an intervertebral disc space for deploying and receiving an artificial spinal implant. The surgical instrumentation and methods of this invention can be adapted for use with a spinal implant having an upper and/or superior component and an inferior and/or lower component which undergo motion relative to each other, while the upper and lower components each engage with their respective adjacent vertebral surfaces. Much of the surgical instrumentation and/or methods described herein can be utilized with a wide variety of spinal implants, including implant components having a stabilizing structure or “keel” extending into channels or other openings created within the vertebral bodies.

Embodiments of the disclosure are generally directed to surgical instruments and devices including distractors, trialing tools, drivers, inserters, alignment guides, etc. The surgical instrumentation may be adapted to permit insertion through a standard open surgical procedure, minimally invasive procedure and/or micro-incision. The surgical instrumentation may be further adapted to permit navigation and/or robotic identification, registration and/or use. The surgical instrumentation can be suitable for spinal surgeries and procedures. Examples of surgical procedures suitable for employing the surgical instrumentation described herein include, but are not limited to, insertion and securement of orthopedic implants, including spinal implants, fasteners (such as screws and caps), or any procedure operating on a patient.

Orthopedic surgeries and related procedures often require removal, repositioning and/or shaping of bone and other tissues. One commonly used tool to cut s to cut or prepare the bone is a reciprocating surgical system. An orthopedic prosthesis or implant may be deployed onto the prepared bone. Different tools with different cutting features can be coupled to a powered handle of the reciprocating system to facilitate cutting through the bone

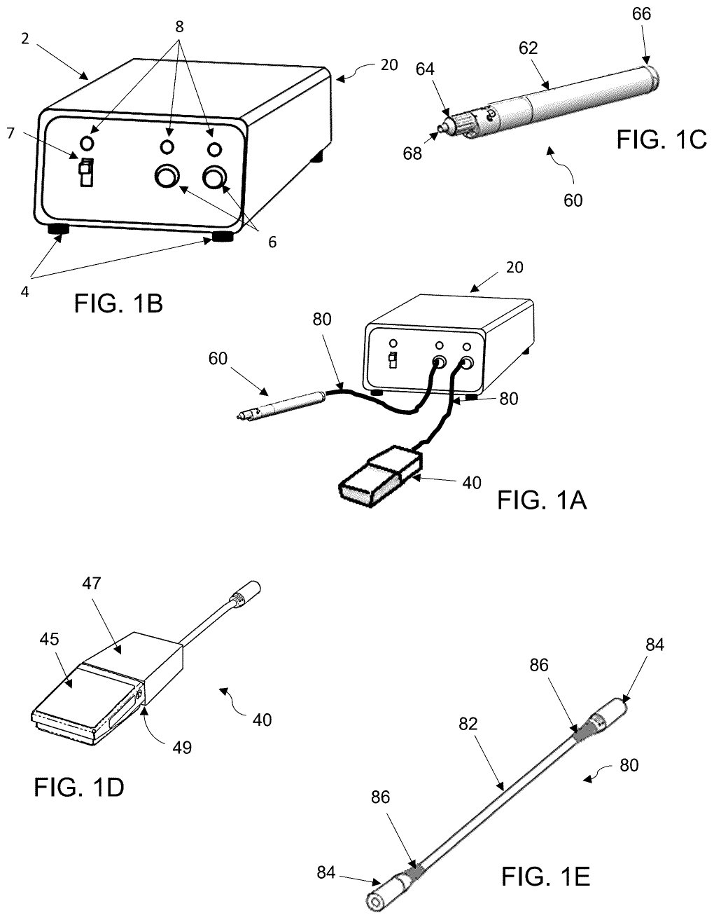

FIGS. 1A-1E depict different views of one exemplary embodiment of a powered surgical cutting and/or shaping system, which includes a control box 20, a footswitch pedal 40, a powered handle 60 and one or more cable connectors 80. As best seen in FIG. 1B, the control box includes an enclosure 2 having a plurality of flexible foot pads 4 extending downwardly from a bottom surface thereof. The foot pads may comprise a flexible material, such as a polymer and/or a rubber, and will desirably help absorb any vibrations exhibited during activation of the system, as well as isolate the enclosure from voltages and/or currents (e.g., static charges) from the underlying support surface. The enclosure 2 may comprise a metallic and/or nonconductive material, which may include a Restriction of the Use of Certain Hazardous Substances in Electrical and Electronic Equipment (RoHS) material, such as a polymer and/or a metal. In one exemplary embodiment, the enclosure may comprise a metal such as aluminum and/or stainless steel. The enclosure may further include various coatings and/or other substances (e.g., antiviral, antibacterial, flame retardant, chemical resistive coatings) and/or desired materials (e.g., thermally conductive and/or dimensionally stable materials). In one exemplary embodiment, the coating may include a clear, chromate coating.

The control box 20 may further include two or more jacks or electrical connectors 6. The two or more electrical connectors may be disposed on various surfaces (e.g., top, front, side and/or back) of the enclosure, and may be utilized to couple the footswitch pedal and/or a powered handle (or other components) to the box. As shown, the connectors 6 can comprise a jack-type connector commonly used in powered surgical components, into which associates plugs (not shown) may be connected. The control box 20 may further include one or more indicators or lights, such as LED light indicators 8. The one or more LED lights 8 may be highlighted to a standard color (or may incorporate multiple colors indicating different plug conditions) when power is supplied to the control box (and/or when a power switch 7 of the control box is activated), when the footswitch pedal is properly connected to the control box and/or when the powered handle is properly connected to the control box.

In one exemplary embodiment the enclosure can comprise 5052 aluminum with a ROHS compliant clear chromate coating, a polyester TGIC powder coat paint with epoxy ink silkscreen printing on the front and top of the enclosure. The front panel of the enclosure may comprise a chrome plated brass housing with red/green LED indicators therein. The connectors may comprise chrome plated brass housings with 4-pin push/pull electrical connector for the footswitch and an 8-pin push/pull electrical connector for the handpiece. A standard three-prong power cord (not shown) can be utilized to provide 110 v and/or 220 v power to the control box and associated components, if desired.

The powered reciprocating system comprises a footswitch pedal 40. The footswitch pedal 40 is desirably placed on a floor of the surgical theatre at a location to be actuated with a surgeon or other user's foot. The surgeon and/or other user can desirably use their foot to control the actuation and/or speed of reciprocation and/or oscillation of the attached handpiece (e.g., utilizing a variable speed control foot pedal or rheostat). The actuation method of the footswitch may comprise a slow action, a snap action and/or a potentiometer. The switch function of the footswitch pedal may comprise (1) maintained contact, (2) applied momentary contact, (3) variable depth for actuation and/or speed control, (4) differential foot placement for single stage, two stage, three stage and/or any combinations thereof. The switch configuration of the footswitch pedal may comprise normally open or normally closed, as desired. The actuation plate or pedal 45 of the footswitch may comprise a single plate (as depicted) or incorporate multi-plate (e.g., split plate) design (not shown), with each pate section providing a different actuation operation.

The footswitch pedal can comprise a footswitch body 49, at least one pedal 45 and a footswitch cover 47. In one exemplary embodiment the footswitch pedal 45 can comprise Pocan 3225 20% glass filled polyester, with a footswitch cover 47 and/or footswitch body 49 of die cast aluminum with a powder coat paint and epoxy ink silkscreen printing thereupon.

As depicted, the at least one pedal is desirably movable from a first position, wherein the reciprocation and/or oscillation is turned off, to a second position, wherein the reciprocation and/or oscillation is actuated and/or activated. The footswitch cover is disposed over a portion of the footswitch body and/or a second end of the footswitch body. The pedal is disposed over a portion of the footswitch body and/or a first end of the footswitch body. The footswitch pedal further comprises at least one cable connector 51. The at least one cable connector 51 extends from the footswitch pedal and has a connecting end 53 which desirably couples to a location on the control box.

The footswitch pedal, footswitch body, at least one pedal and/or a footswitch cover can comprise a material, such as a composite polymer, a polymer and/or a metal (e.g., die cast aluminum). The polymer may include thermoset or thermoplastic polymers, while the POCAN may further comprise a glass filled polyester. The footswitch pedal may further incorporate protection features such as partial (or no) activation guards, a full guard or interlock switch, an anti-trip mechanism, a dustproof cover, an explosion proof housing, and/or weather resistant or waterproof elements.

A cable 80 can be connected between the footswitch and the enclosure, as well as between the enclosure and the handpiece. One exemplary cable suitable for use in either or both applications, such as depicted in FIG. 1E, can include a silicone sheathed flexible cable body 82, with proximal and distal connectors 84 (e.g., chrome plated copper alloy housing 4-pin- or 8-pin-push-pull electrical connectors) and optional silicone strain reliefs 86.

FIG. 1C depicts a powered handpiece 60, which comprises a handpiece body 62 having a first end 64, a second end 66 and a connection mechanism 68. The connection mechanism 68 can comprise an inner cavity disposed at the first end, wherein the inner cavity is shaped and/or configured to accommodate or receive a shaft of a replaceable surgical instrument or tool. In various embodiments, a connection mechanism may include one or more connection features for mating or engaging with corresponding connection features of one or more shafts of surgical instruments. The connection features can provide a removably secure connection, desirably preventing the surgical tool from becoming disengaged from the further component during use in a surgical procedure. The connection mechanisms can include male and/or female connectors. If desired, the connection mechanism may incorporate a Hudson connection, an Association for Osteosynthesis (AO) connection, a Trinkle connection and/or others well known in the art. A cable connector (not shown) can be coupled to powered handpiece, with an opposing end of the cable coupled to the control box.

In one alternative embodiment, an inner cavity of the powered handpiece may incorporate a Hudson connection mechanism, with the inner cavity having a flat portion shaped to engage another flat portion formed by a profile of an end of the shaft. The inner cavity may further include one or more ball detents to engage with a channel or recess at the end of the shaft. In this embodiment the Hudson mechanical connection desirably provides a relatively accurate connection in relation to the shaft length, and may include one or more locking positions over 360 degrees.

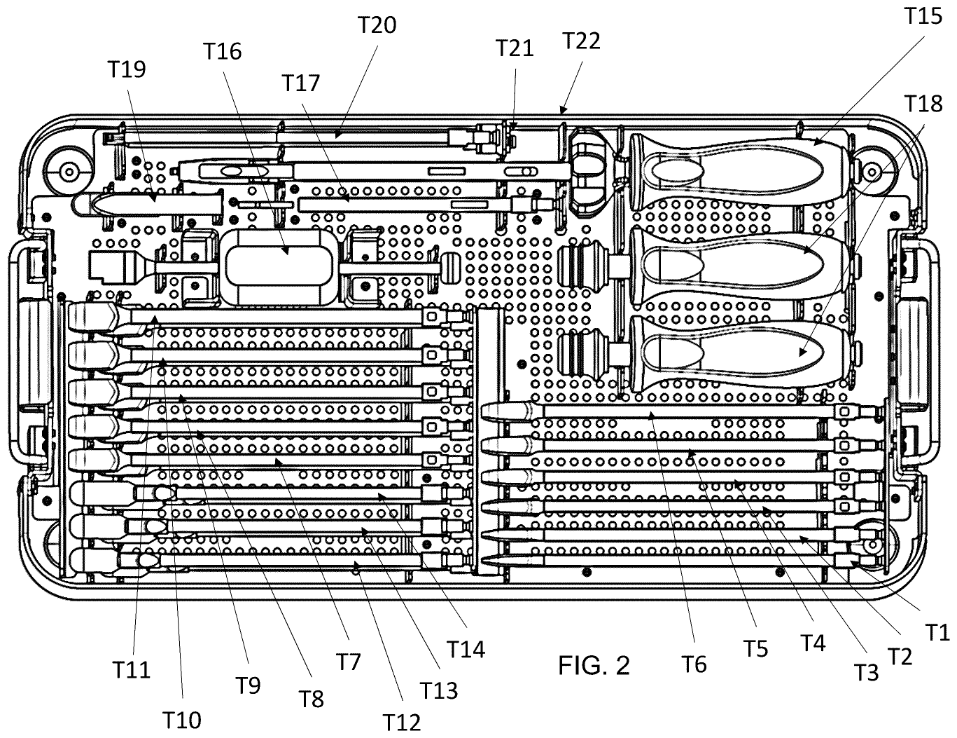

In various embodiments, a surgical instrumentation kit may be provided which comprises a variety of available tools for use throughout the procedure (as shown in FIG. 2). For example, at least three length trials may be provided in the kit, which trials desirably match and/or approximate the lengths of the superior component of the implant. The at least three length trials comprise a short (25 mm), a medium (29 mm), and a long (32 mm). Accordingly, the surgeon may attach the length trial tool to a standard handle known in the art for easier manual manipulation and/or the surgeon may attach the length trial to a custom handle, e.g., the Hudson handle, for easier manual manipulation. Desirably, the tools depicted herein are reusable and re-sterilizable, although various of the tools may be disposable, if desired.

TABLE 1
Exemplary kit components / tools of FIG. 2.

| ITEM No. | Description |
|---|---|
| T1 | Distractor, 5 |
| T2 | Distractor, 6 |
| T3 | Distractor, 7 |
| T4 | Distractor, 8 |
| T5 | Distractor, 9 |
| T6 | Distractor, 10 |
| T7 | Height Trial, 11 |
| T8 | Height Trial, 12 |
| T9 | Height Trial, 13 |
| T10 | Height Trial, 14 |
| T11 | Height Trial, 15 |
| T12 | Length Trial, Short |
| T13 | Length Trial, Medium |
| T14 | Length Trial, Long |
| T15 | Inserter |
| T16 | Slap Hammer |
| T17 | Removal Tool |
| T18 | Hudson Handle |
| T19 | Screw Guide |
| T20 | Screwdriver |
| T21 | Removal Driver |
| T22 | Base |
| T23 | Lid (not shown) |
| T24 | IFU (U.S.) (not shown) |

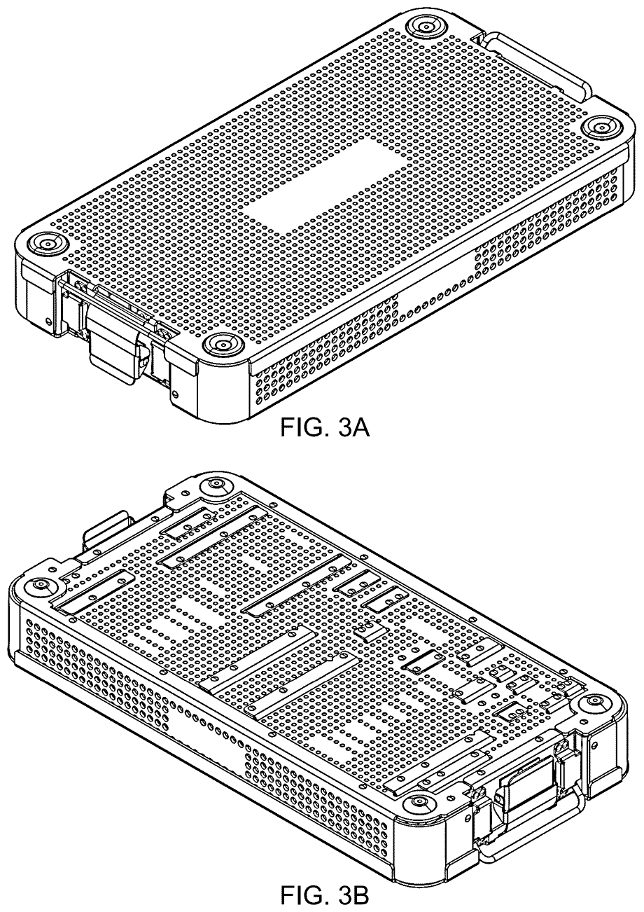

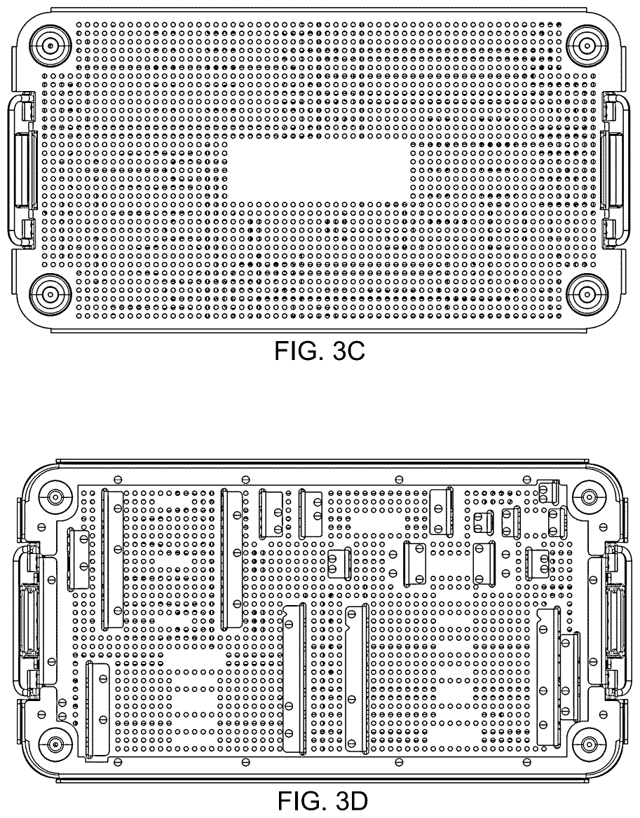

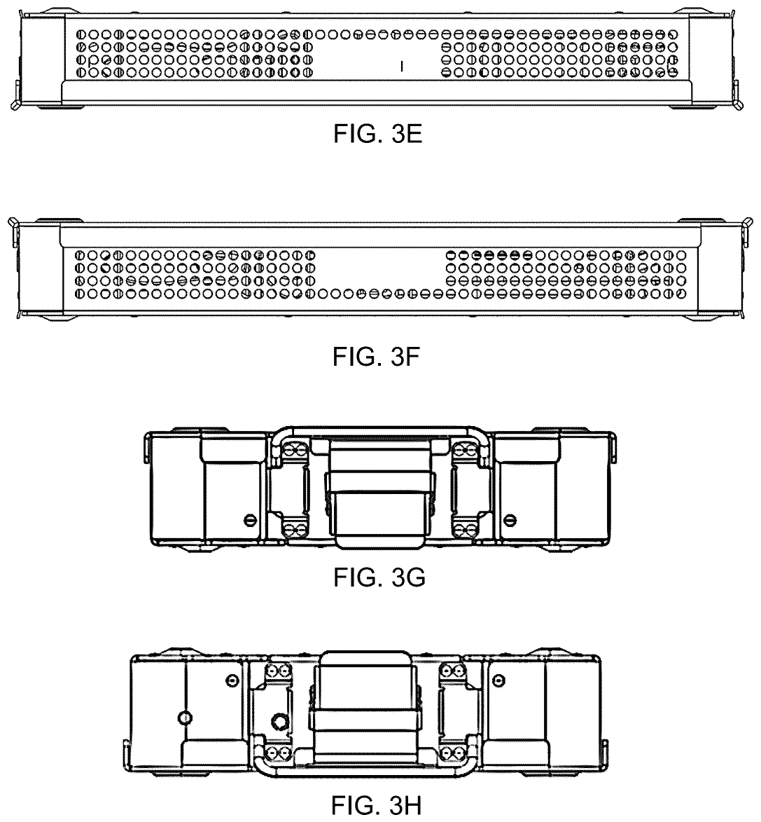

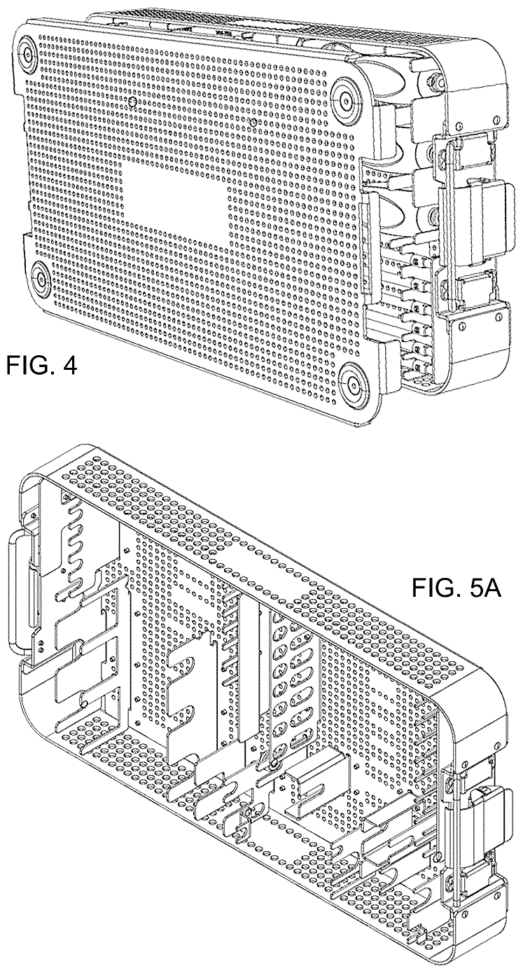

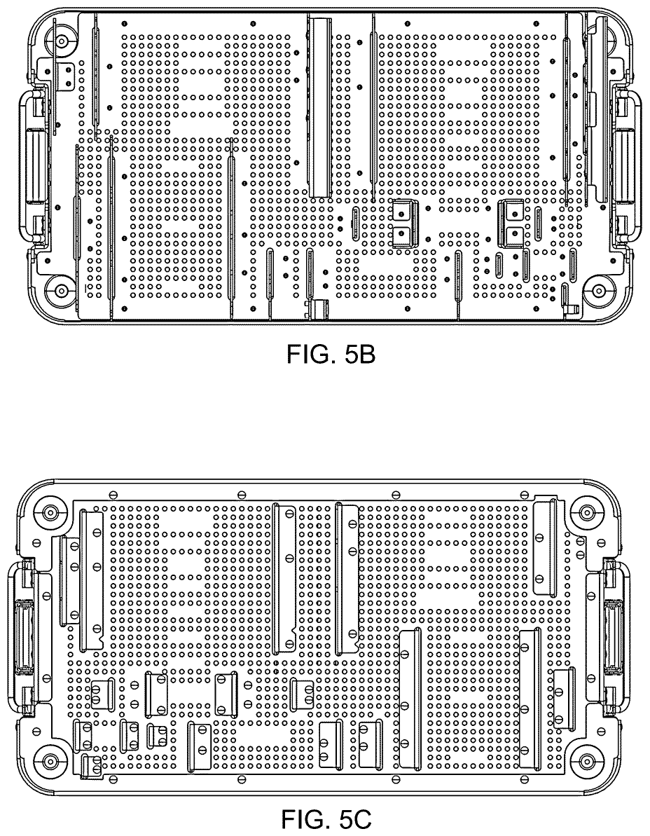

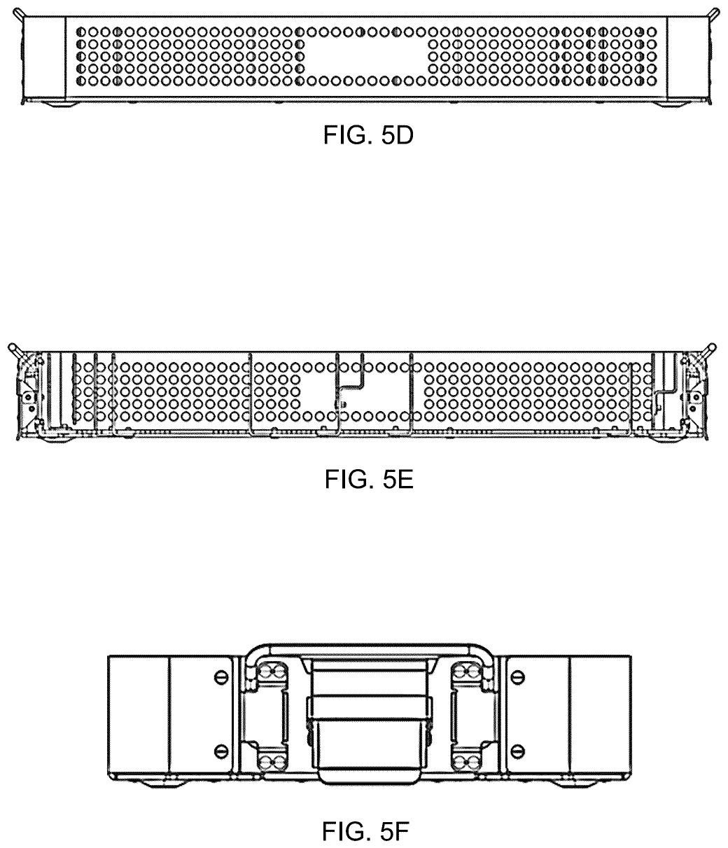

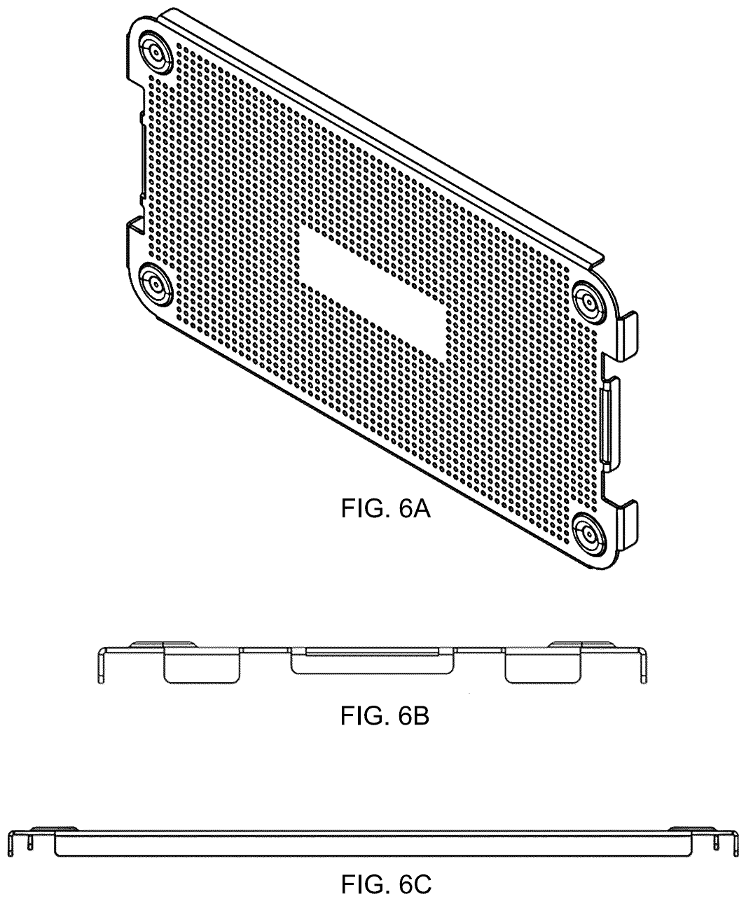

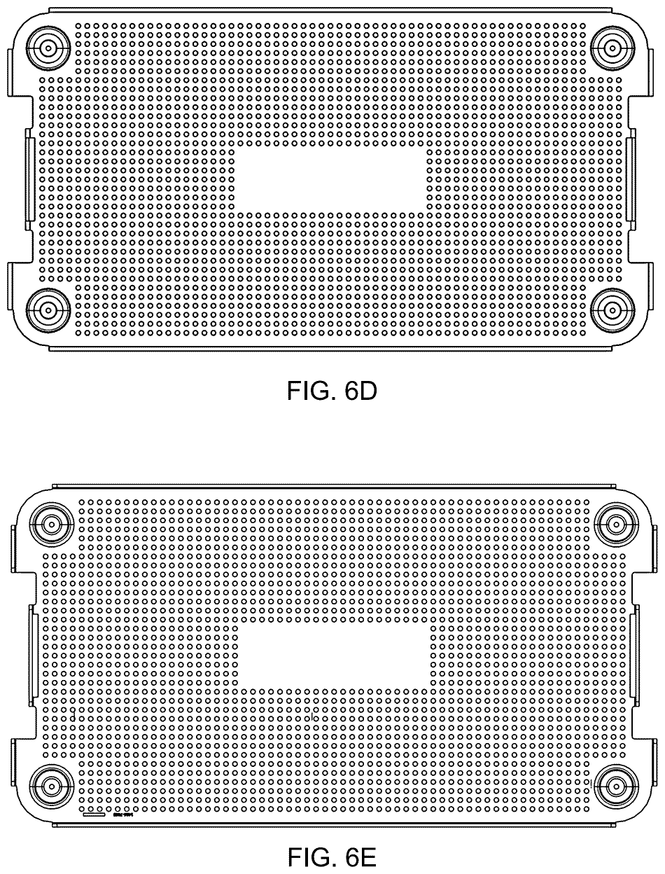

FIGS. 3A through 3H depict various views of a surgical instrument case and case lid, with the case lid in a secured positioned on the case. FIG. 4 depicts an exploded view of the surgical instrument case and case lid of FIGS. 3A-3H. FIGS. 5A-5F depict various views of the surgical instrument case without the case lid, and FIGS. 6A-6E depicts various views of the case lid.

The surgical instrument kit can desirably comprise a modular tray or container system, having at least one case (or base) and a lid. In the disclosed embodiment, the case can further comprise a plurality of dividers and/or other holding elements, which in some embodiments may be user configurable, adjustable and/or removeable. In some embodiments, the case may contain one or more removable/modular trays within a single base. As best seen in FIGS. 5A and 5B, a plurality of dividers and/or retaining elements are coupled to the base and extend into the case to hold, secure and/or separate each of the surgical instruments. In some cases the dividers or other elements can comprise one or more structure having notches, wherein the notches can be sized and configured to receive a portion of the plurality of surgical instruments. As depicted, each of the one or more notches can be spaced apart and axially aligned. The modular tray system may further comprise one or more handles for ease of transport. The modular tray system may further comprise one or more locking mechanisms to ensure the tools and/or the case lid are secured. The lid may be disposed over a portion of the base and coupled to the base.

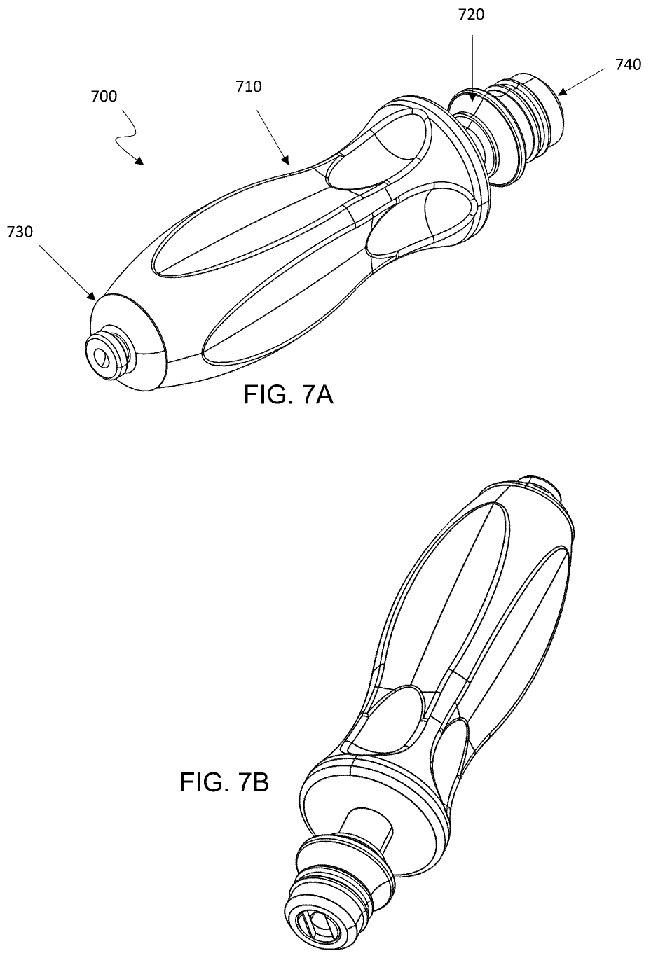

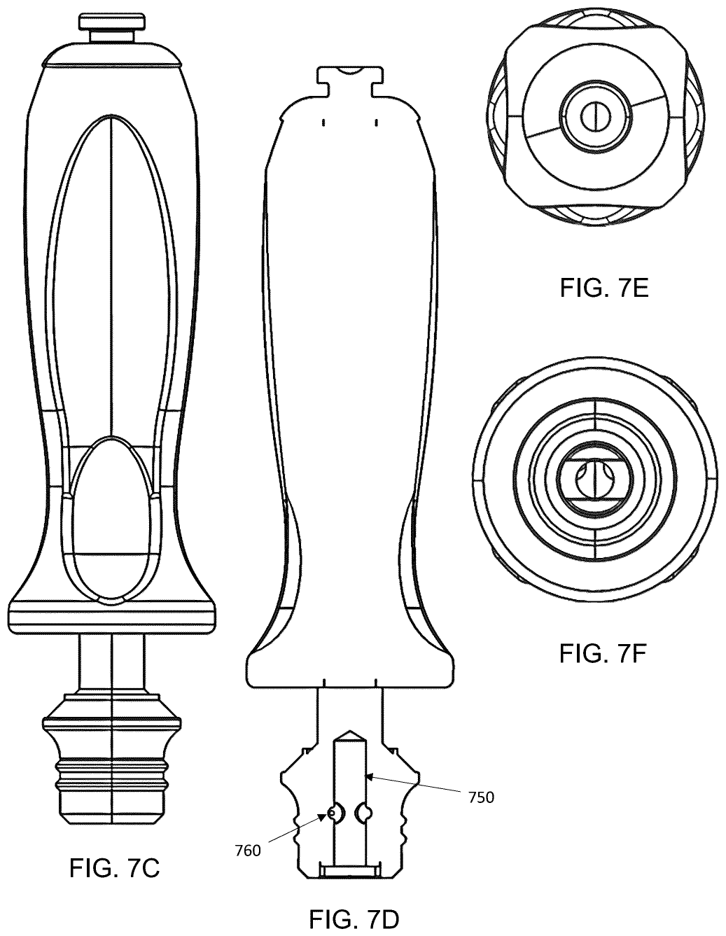

FIGS. 7A-7F depict different plan views of one embodiment of a modular handle 700 for use with various of the surgical instruments within the kit. The handle 700 can comprise a body 710 having a first end 720, a second end 730 and a connection mechanism 740. The connection mechanism 740 can be disposed at the first end of the body (and/or the second end, as desired). AS best seen in FIG. 7D, the connection mechanism can comprise an inner cavity 750, the inner cavity shaped and configured to accommodate or receive a shaft of a replaceable surgical instrument or tool. The connection mechanism can desirably include one or more connection features for mating or engaging with corresponding connection features of one or more shafts of surgical instruments, such as corresponding cavity/tool shapes, as well as engagement and/or locking mechanisms such as a detent mechanism 760 or similar features. The connection features can provide a removably secure connection, preventing the surgical tool from becoming disengaged from the further component during use in a surgical procedure. The connection mechanisms can desirably include male and/or female connectors as well as other well known tool securing arrangements. Exemplary connection mechanisms can include a Hudson connection, an Association for Osteosynthesis (AO) connection and/or a Trinkle connection. The connection mechanisms comprise quick-connect and quick-disconnect.

The outer surface of the body 710 can comprise a variety of shapes and/or configurations, which desirably include a generally cylindrical and/or elliptical form (on at least some portion of the body) to accommodate handling and/or manipulation by the user's hand or hands. If desired, the body may comprise a generally uniform shape and/or non-uniform shape.

As depicted in FIG. 7A, the outer surface further can comprise one or more flattened or textured surfaces which desirably allow the user to utilize their fingers/thumb and/or palm to straighten, press-on and/or guide the tool for better positioning, as well as avoid unwarranted twisting or rotation of the tool during use. The shape may further comprise ergonomic shaping. The body and/or outer surface may further comprise one or more flutes or texturing. The one or more flutes may be disposed along the longitudinal axis and/or the horizontal axis of the body. The one or more flutes may be positioned radially about a central axis of the interface. The positioning may comprise symmetric or asymmetric positioning. The positioning may further include 90 degrees apart.

In one exemplary embodiment, the handle can comprise a body diameter in a range of 25 to 45 mm, more preferably in a range of 35 to 40 mm. The body length can comprise 75 mm to 125 mm, more preferably a range of 75 mm to 100 mm and/or a range of 100 mm to 115 mm. In various other embodiments, a total handle length can be from 75 mm to 145 mm, preferably 100 mm to 145 mm, more preferably 125 mm to 145 mm, and even more preferably 140 mm to 145 mm.

The handle body can comprise a body material, while the connection mechanism can comprise a connection material, which may be the same or different material than the body material. Desirably, the body material will provide for sufficient frictional resistance between a user's or surgeon's hand and the handle to allow for proper use and manipulation of the handle. This is particularly important where a considerable force must be applied with a sweaty hand. The body material should be made of non-slip, non-conductive and compressible materials, and/or may include texturing thereof. In one embodiment, the body material may comprise a rubber or rubberized material. The rubber(ized) material can desirably provide a good grip, thereby reducing any effort needed to use the tool effectively, and desirably prevent the tool from slipping out of the hand. The body material may further comprise electrical and/or heat insulative properties. Depending upon a variety of factors, the body material and/or the connection material mat include a rubber, a polymer, a metal and/or various combinations thereof. Where a polymer is utilized, it may include thermoset, thermoplastic, thermoset elastomers, and/or thermoplastic elastomers.

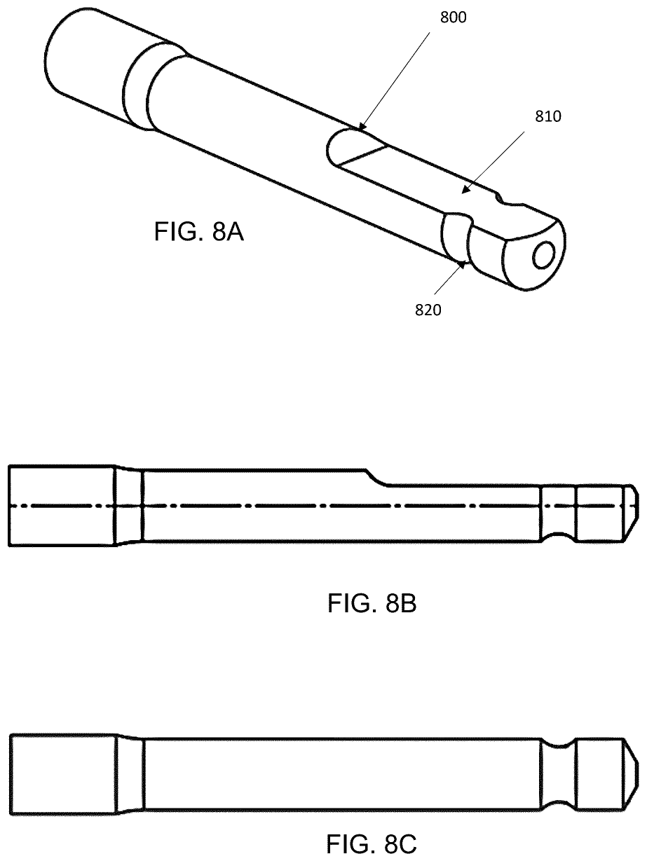

FIGS. 8A-8C depicts different views of one exemplary embodiment of a shaft 800 having an AO-type shaft connection 800. An AO connection mechanism is configured to receive an end of a shaft (e.g., the “male” end) of a surgical instrument or tool having a D-shaped profile. A flat portion 810 defined by the D-shaped profile of engages another flat portion (referred to herein as the “AO flat”) formed in an inner cavity 750 (e.g., the “female” end) of an opposing or corresponding connection mechanism. This engagement desirably prevents rotation of the shaft with respect to the interface. The shaft may be retained in the interface by the operation of one or more ball bearings. Two ball bearings or detents 760 (or similar retaining features) are generally positioned radially about a central axis of the interface, about 180 degrees apart. The ball bearings may engage a groove 830 formed in the end of the shaft. The ball bearings are often held by a substantially straight walled portion of the sleeve. The AO connection mechanism can desirably include at least one locking position which is effective over 360 degrees. The AO connection mechanism may further comprise a variety of materials, such as a rubber, metal and/or polymer material and/or various combinations thereof.

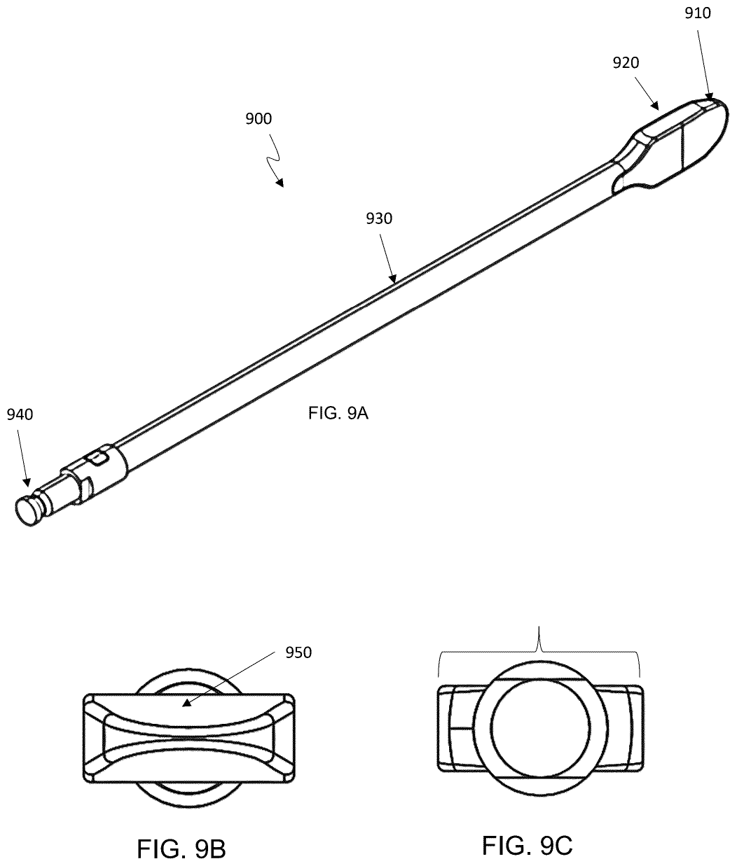

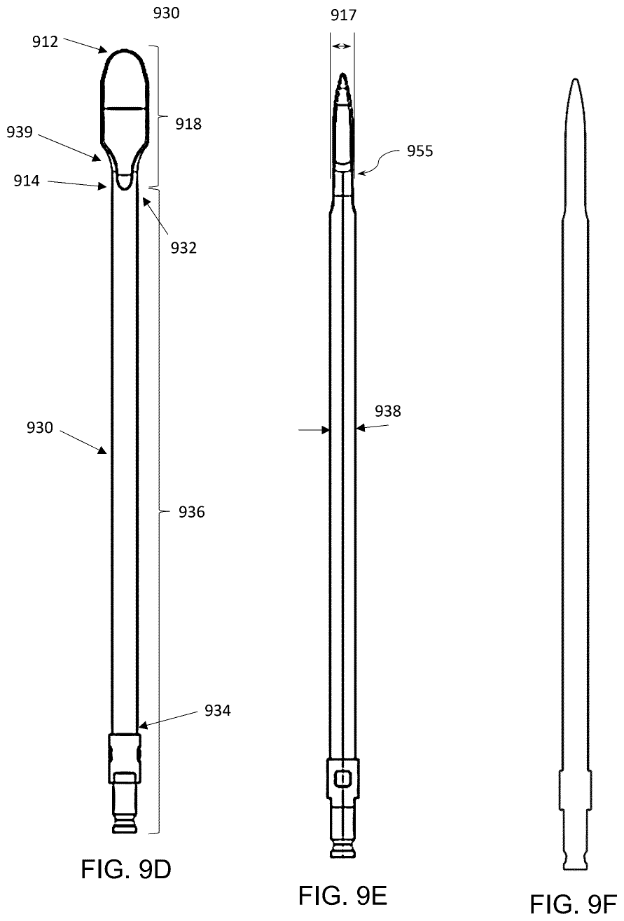

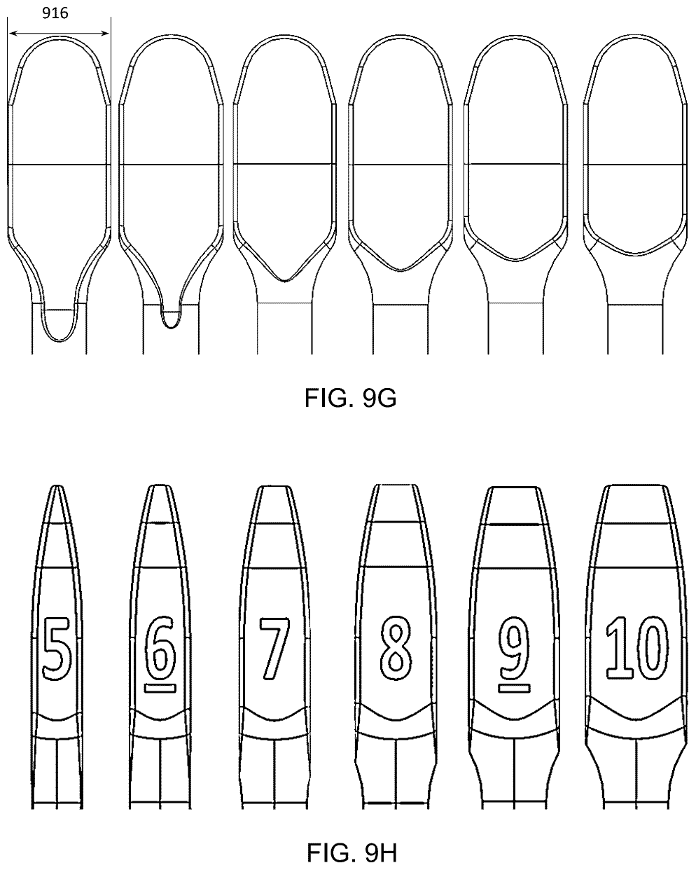

FIGS. 9A-9F depicts different views and a cross-sectional view of one embodiment of a distractor tool 900. The distractor tool 900 is employed to urge two adjacent vertebral bodies apart and to maintain the vertebral bodies in a selected spatial relationship with regards to each other. The distractor tool comprises a head 910, tip or body 920, a shaft 930, and a connector 940. FIGS. 9G-9H depicts front and side views of the various available sizes of the distractor tool of FIGS. 9A-9F.

As best seen in FIGS. 9D, 9E and 9G, each distractor head 910 includes a first end 912, a second end 914, a width 916, a height 917 and a length 918. In various embodiments, the length of the head can range from 25 to 35 mm; while in other embodiments the length may be 25 to 30 mm, while in other embodiments the length may be 30 to 35 mm; and/or in some embodiments the length may be 31 to 33 mm. In the exemplary kit of FIGS. 9G and 9H, the height or depth of the different head components may a set of sequential increasing height or depth, including starting at 5 mm or greater with 1 mm increments (e.g., 5 mm, 6 mm, 7 mm, etc.). The height or depth of the head may be approximately 5 to 15 mm and/or the height may be 5 to 10 mm, with various indicators of tool dimensions desirably provided thereon. The height or depth of the head may comprise at least a portion of uniform height along the longitudinal axis of the head and/or at least a portion of a non-uniform height along the longitudinal axis of the head. The height or depth of the head may comprise at least a portion of a uniform height along the length of the head and/or at least a portion of a non-uniform height along the length of the head. The non-uniform length may comprise at least a portion of the length that is tapered. The width of the head may match or substantially match the width of a spinal implant. If desired, the widths of each of the heads may stay constant as the height and/or depth changes.

In various embodiments, the head may further comprise a plurality of heights, such as a first height and/or a second height. The second height disposed at a second end, and the first height disposed at a first end of the head. The second height may be greater than the first height. The second height may comprise a height of 5 to 10 mm, that decreases to 50 percent or greater height at the first end compared to the second height to create a tapered end to facilitate insertion of head into the intervertebral disc space and urge the vertebral bodies apart into a distracted state. When used anteriorly, the head may be generally of a lesser height at the leading end or first end of the head, which can be advantageous to penetrate the space between the vertebral bodies (such as where the formation of rim osteophytes may have created a reduced separation or “fish mouth” condition). The increase in height, from first height to second height also may be used to position each of the two adjacent vertebral bodies in an angular spatial relationship, such as to create a desired lordosis. The head may be solid or hollow. The head may comprise a shape, the shape may include generally rectangular shape, a round-rectangular shape, an oval shape and/or an elliptical shape. The head may further comprise a taper 950 disposed on a first end.

As best seen in FIG. 9E, the distractor and/or head may further comprise one or more reference markers 955. The reference markers may be used (in combination with a fluoroscope, for example) when the distractor is inserted into the intervertebral disc space to help guide the surgeon and/or user on positioning of the distractor relative to the intervertebral disc space. The one or more reference markers may be confirmed radiographically to determine the length of insertion of the head of the distractor into the intervertebral disc space to achieve the desired distraction and vertebral alignment. The one or more reference markers may comprise a radiopaque material or be radiopaque to allow visualization with an imaging equipment. Furthermore, the one or more reference markers may have its position confirmed radiographically to ensure that the head is accurately positioned within the intervertebral disc space and to assess the depth of the intervertebral disc space relative to the known length of the head.

The distractor may comprise a shaft 930 having a first end 932, a second end 934, a length 936, and/or a diameter 938. The diameter of the shaft may be in the range of 5 mm to 8 mm, of 6 mm to 8 mm, and/or of 6 mm to 7 mm. The diameter of the shaft may be uniform along the longitudinal axis and/or length of the shaft. The shaft may comprise a shape, the shape may be cylindrical or generally cylindrical. The length of the shaft may comprise a range of 130 mm to 150 mm, a range of 135 mm to 145 mm, and/or a range of 140 mm to 145 mm. The head is desirably disposed at a first end of the shaft, and the connector disposed at a second end of the shaft. The junction of the first end of the shaft and the head may form an tapered and/or atraumatic shoulder 939 that abuts or contacts against tissue or bone when the head is inserted between the two adjacent vertebral bodies. The shaft may be solid or hollow.

The connector of the shaft may include a male and/or a female connector. The connector may be configured to engage with a Hudson connection, an Association for Osteosynthesis (AO) connection and/or a Trinkle connection. The connection mechanism may comprise quick-connect and/or quick-disconnect elements. The connector may be disposed within and/or coupled to a manual handle and/or a powered handle or handpiece.

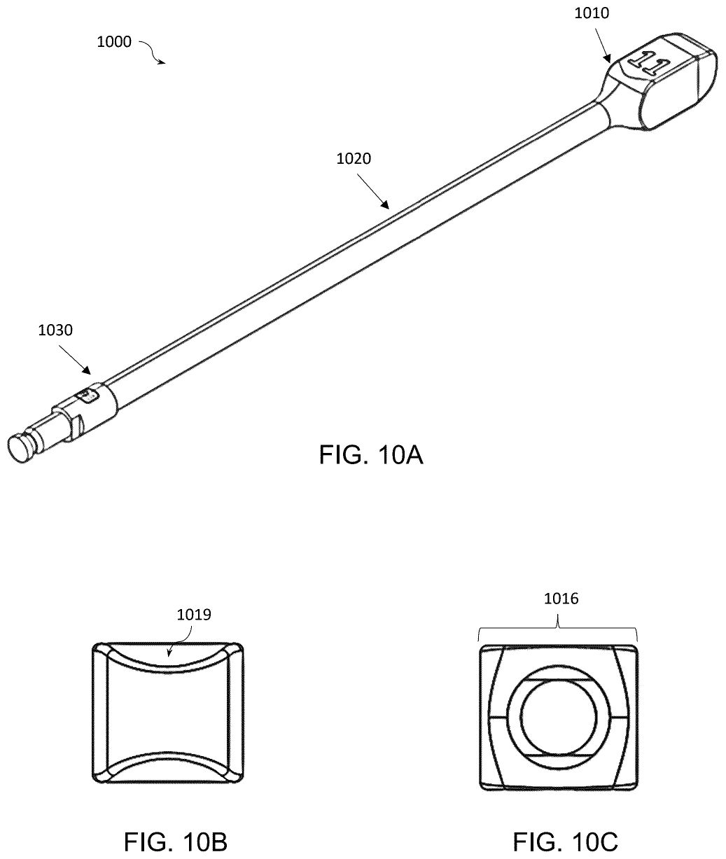

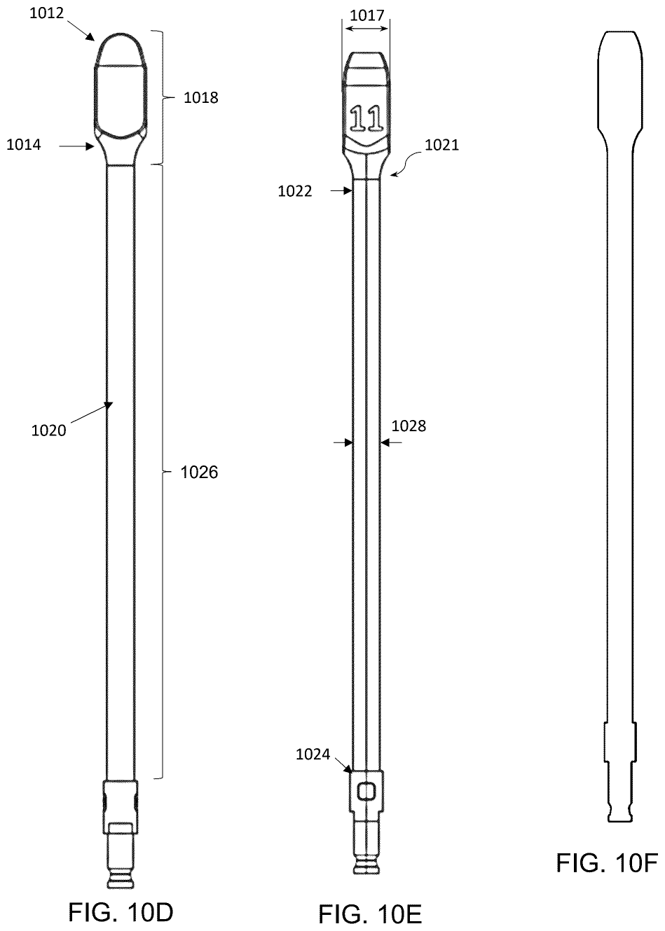

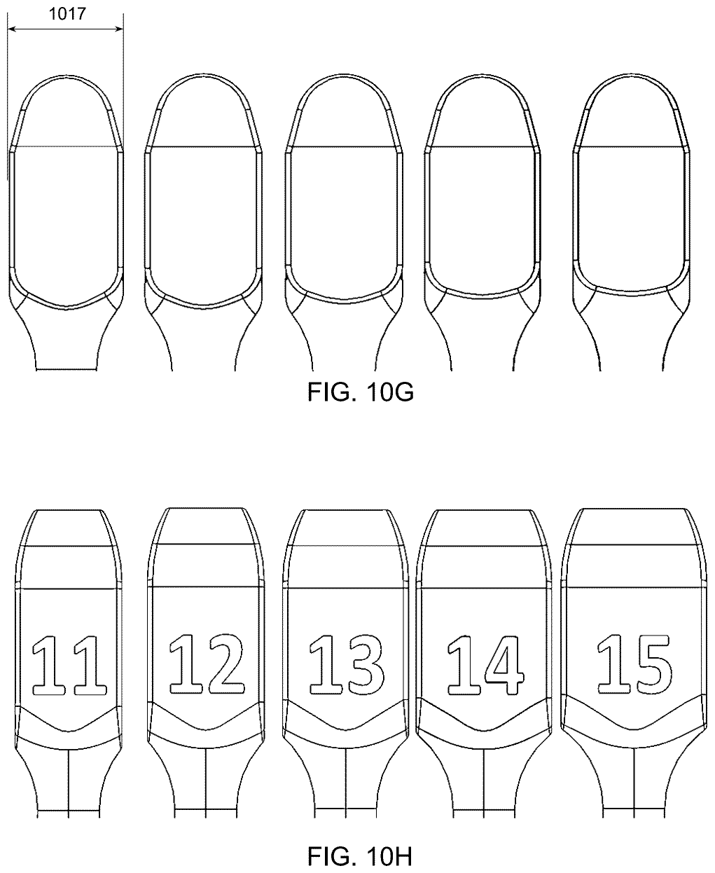

FIGS. 10A-10F depicts different plan views and a cross-sectional view of an exemplary embodiment of a height trial tool 1000 and related tool kit. Each height trial tool 1000 can desirably urge two adjacent vertebral bodies apart and maintains the vertebral bodies in a selected spatial relationship to each other to be able to determine an approximated or proper spinal implant height while maintaining proper tissue tension at and/or near the intervertebral disc space. The height trial tool 1000 comprises a head, tip or body 1010, a shaft 1020, and a connector 1030. FIGS. 10G-10H depicts a front and side view of the various available sizes of the height trial tool of FIGS. 10A-10F.

The head of the height trial comprises a first end 1012, a second end 1014, a width 1016, a height 1017 and a length 1018. The length of the head comprises approximately 25-35 mm; the length of the head may further comprise 25-30 mm; the length of the head may further comprise 30-35 mm; and/or the length of the head may further comprise 28-31 mm. The height or depth of the head comprises sequential increasing height or depth, including starting at 11 mm or greater with 1 mm increments. The height or depth of the head comprises approximately 10 to 20 mm and/or the height may further comprise 11 to 15 mm. The height or depth of the head comprise at least a portion of uniform height along the longitudinal axis of the head and/or at least a portion of a non-uniform height along the longitudinal axis of the head. The height or depth of the head comprise at least a portion of a uniform height along the length of the head and/or at least a portion of a non-uniform height along the length of the head. The non-uniform length may comprise at least a portion of the length that is tapered and/or truncated tapered. The height or depth of the head may match or substantially match the total height of a spinal implant. The width of the head may match or substantially match the width of the spinal implant. The width of each of the heads may be not change as the height or depth changes.

The head of the height trial may further comprise a first height and/or a second height. The second height disposed at a second end, and the first height disposed at a first end of the head. The second height is greater than the first height. The second height comprises a height of 11 mm to 15 mm, that decreases to 25 percent or greater mm at the first end compared to the second height to create a tapered end to facilitate insertion of head into the intervertebral disc space and urge the vertebral bodies apart into a distracted state. When used anteriorly, head is generally of a lesser height at the leading end or first end of the head. The increase in height, from first height to second height also may be used to position each of the two adjacent vertebral bodies in an angular spatial relationship, such as to create lordosis. The head may be solid or hollow. The head may comprise a shape, the shape may include generally rectangular shape, a round-rectangular shape, an oval shape and/or an elliptical shape. The head may further comprise a taper 1019 and/or a truncated taper disposed on a first end.

The height trial tool and/or head of the height trial tool may further comprise one or more reference markers 1021. The reference markers are used when the height trial is inserted into the intervertebral disc space to help guide the surgeon and/or user on positioning of the height trial tool relative to the intervertebral disc space. The one or more reference markers may be confirmed radiographically to determine the length of insertion of the head of the height trial tool into the intervertebral disc space to achieve the desired height, desired tension and vertebral alignment. The one or more reference markers may comprise a radiopaque material or be radiopaque to allow visualization with an imaging equipment. Furthermore, the one or more reference markers may have its position confirmed radiographically to ensure that the head of the height trial tool is accurately positioned within the intervertebral disc space and to assess the depth of the intervertebral disc space relative to the known length of the head of the height trial tool. The one or more reference markers may comprise a laser marking, a ring, and/or an opening. The one or more reference markers may be disposed onto a portion of the head and/or the shaft.

The height trial tool may comprise a shaft 1020. The shaft comprises a first end 1022, a second end 1024, a length 1026, and/or a diameter 1028. The diameter of the shaft may comprise a range of 5 mm to 8 mm; the diameter may further comprise a range of 6 mm to 8 mm; and/or the diameter may further comprise a range of 6 mm to 7 mm. The diameter of the shaft may be uniform along the longitudinal axis and/or length of the shaft. The shaft may comprise a shape, the shape may be cylindrical or generally cylindrical. The length of the shaft may comprise a range of 130 mm to 150 mm; the length of the shaft may comprise a range of 135 mm to 145 mm; and/or the length of the shaft may comprise a range of 140 mm to 145 mm. The head is disposed at a first end, and the connector disposed at a second end. The junction of first end of the shaft and head forms an atraumatic shoulder that abuts or contacts against tissue or bone when the head is inserted between the two adjacent vertebral bodies. The shaft may be solid or hollow.

The height trial tool may include a connector 1030, such as a male and/or a female connector. The connectors may be configured to engage with a Hudson connection, an Association for Osteosynthesis (AO) connection and/or a Trinkle connection. The connection mechanisms may comprise a quick-connect and/or quick-disconnect. The connector may be disposed within and/or coupled to a manual handle and/or a powered handle or handpiece.

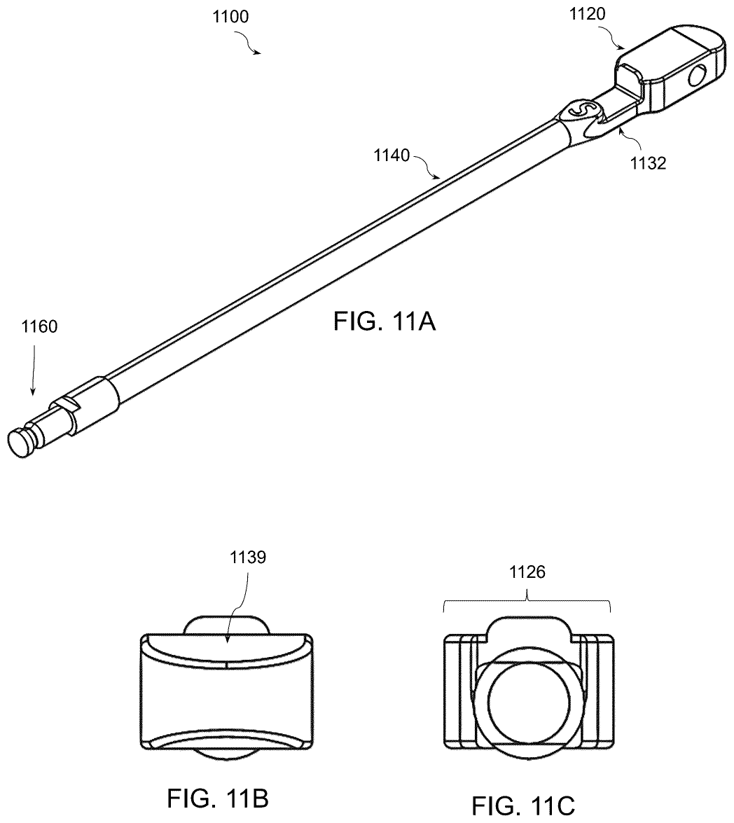

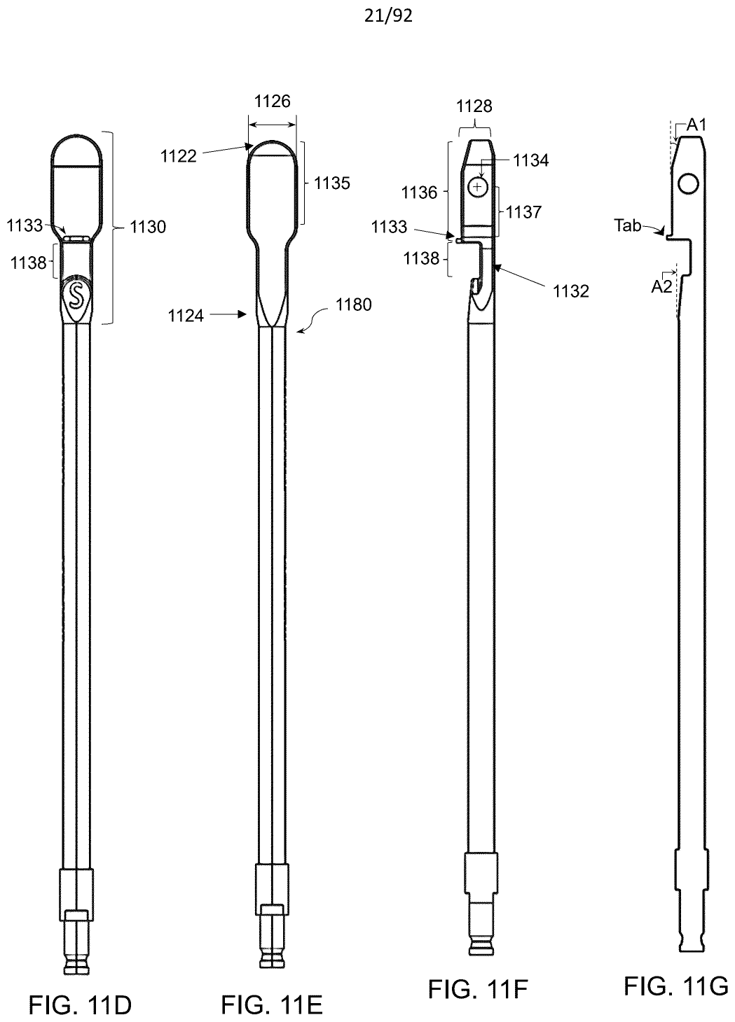

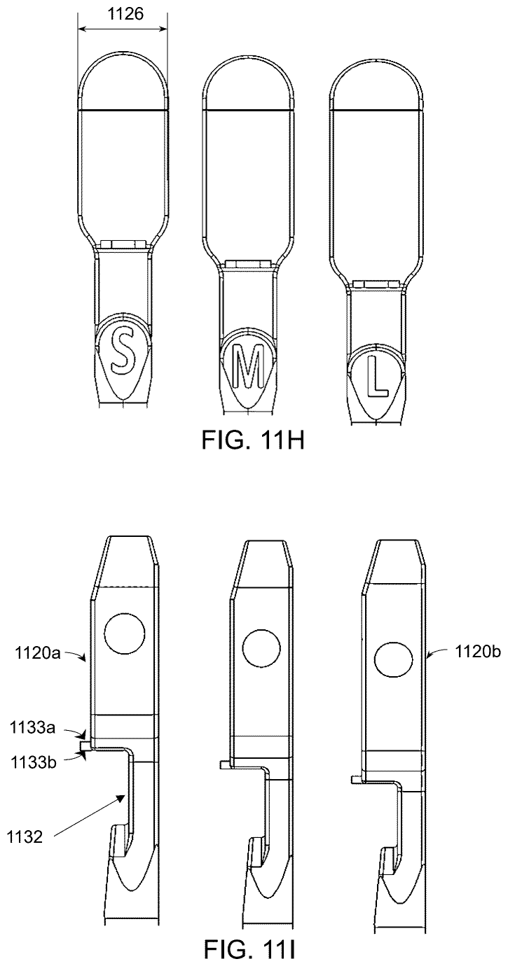

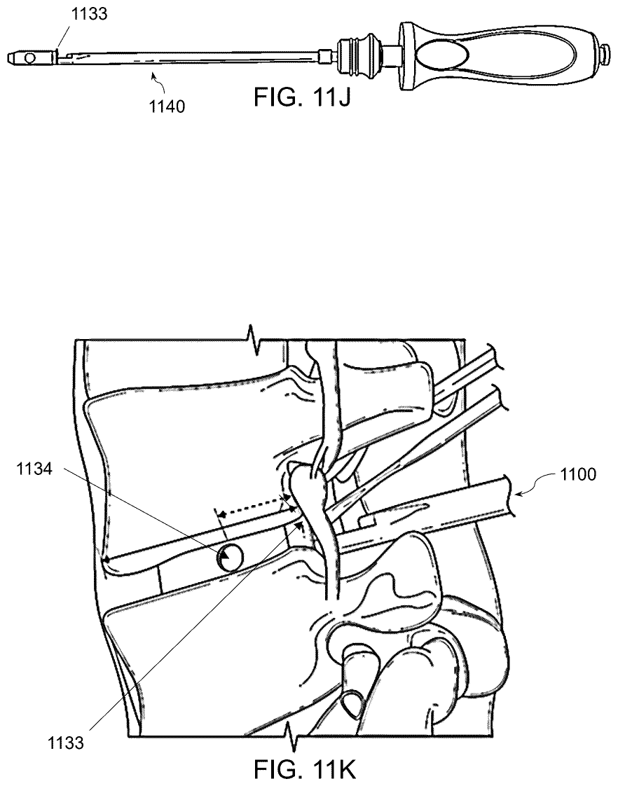

FIGS. 11A-11G depict different plan views and a cross-sectional view of embodiment of a length trial tool 1100. The length trial tool will desirably be utilized to urge two adjacent vertebral bodies apart and maintain the vertebral bodies in a selected spatial relationship to each, while allowing the physician to determine an approximated or proper spinal implant length within and/or near the intervertebral disc space. The length trial tool comprises a head, tip or body 1120, a shaft 1140, a connector 1160 and a reference marker 1180. FIGS. 11H-11I depicts a front and side view of the various available sizes of the length trial tool of FIGS. 11A-11G.

The head 1120 of the length trial comprises a first end 1122, a second end 1124, a width 1126, a height 1128 and a total length or first length (L1) 1130. The head of the length trial further comprises a bridge 1132, a stop tab 1133 and/or a center of rotation (COR) marker 1134. The head of the length trial further comprises a transition length (L2) 1135, tab-to-end length (L3) 1136, a tab-to-opening length (L4) 1137, a bridge opening length (L5) 1138. The total length, L1, of the head may comprise a range 40 to 60 mm; a range of 45 to 55 mm; and/or a range of 45-53 mm.

In one embodiment, the head of the length trial may comprise the dimensions listed within Table 2 for different sizes. For example, the length trials may comprise a short length trial (L3, 25 mm), a medium length trial (L3, 29 mm) and/or a long length trial (L3, 33 mm). The tab-to-end length, L3, may match or substantially match the length of a superior component of a spinal implant. The total length, L1, may match or substantially match the inferior component of a spinal implant.

TABLE 2
Length Trial Dimension Embodiments

| | L1 | L2 | L3 | L4 | L5 | Width | Height |
|---|---|---|---|---|---|---|---|
| Tool Description | (mm) | (mm) | (mm) | (mm) | (mm) | (mm) | (mm) |
| Length Trial, Short | 45.0 | 21.0 | 25.2 | 12.7 | 8.75 | 12.0 | 8.0 |
| Length Trial, Medium | 49.0 | 25.0 | 29.2 | 14.4 | 8.75 | 12.0 | 8.0 |
| Length Trial, Long | 53.3 | 29.0 | 33.5 | 16.0 | 8.75 | 12.0 | 8.0 |

The height or depth of the head of the length trial comprises sequential increasing height or depth, the starting depth at 8 mm or greater and having increasing increments of 1 mm. Alternatively, the height or depth of the head of the length trial may comprise the same height or depth at each of the length trial tool length sizes. The height or depth of the head of the length trial comprise at least a portion of uniform height along the longitudinal axis of the head and/or at least a portion of a non-uniform height along the longitudinal axis of the head. The height or depth of the head of the length trial comprise at least a portion of a uniform height along the length of the head and/or at least a portion of a non-uniform height along the length of the head. The non-uniform length may comprise at least a portion of the length that is tapered and/or truncated tapered. The height or depth of the head of the length trial may match or substantially match the total height of a spinal implant, the spinal implant including an inferior and superior component. Alternatively, the height or depth of the head of the length trial may match or substantially match the superior component or the inferior component of a spinal implant. The width of the head may match or substantially match the width of the spinal implant. The width of each of the heads may be not change as the height or depth changes.

The head of the length trial may further comprise a first height and/or a second height. The second height disposed at a second end and/or adjacent to the tab, and the first height disposed at a first end of the head. The second height is greater than the first height. The second height comprises a height of 8 mm or greater, that decreases at a first angle (A1) of 15 degrees at the first end compared to the second height to create a tapered end 1139 to facilitate insertion of head into the intervertebral disc space and urge the vertebral bodies apart into a distracted state. When used anteriorly, the head of the length trial is generally of a lesser height at the leading end or first end of the head. The increase in height, from first height to second height also may be used to position each of the two adjacent vertebral bodies in an angular spatial relationship, such as to create lordosis. The head may be solid or hollow. The head may comprise a shape, the shape may include generally rectangular shape, a round-rectangular shape, an oval shape and/or an elliptical shape. The head may further comprise a taper and/or a truncated taper disposed on a first end.

The head of the length trial tool may further comprise a tab 1133. The tab 1133 can be used as a positive stop during insertion and/or translation within the intervertebral space of the length trial tool during surgery. The tab 1133 controls of the insertion depth of the length trial tool so as to prevent the surgeon from proceeding to far anteriorly within the intervertebral space and helps determine the position (anterior-to-posterior distance) as it is intended to be deployed as a spinal implant. The tab can comprise an anterior facing surface or a first surface 1133a and/or a posterior facing surface or second surface 1133b. The tab extends from a first contact surface 1120a of the head. The tab extends superiorly from the first contact surface of the head. The tab is disposed near the second end of the head of the length trial. The tab is disposed between the first end and the second end of the length trial head. At least a portion of the anterior facing surface 1133a contacts a portion of the dorsal aspect of the superior apophyseal ring and/or a portion of the posterior facing surface of a superior vertebral body, as shown in FIG. 11K.

The head of the length trial tool may further comprise a bridge 1132. The bridge extends from the tab towards the posterior direction. The bridge having a bridge opening length 1138 which matches or substantially matches a corresponding bridge opening length of an inferior component of a spinal implant. The bridge 1132 is intended to confirm the proper seating or positioning of the bridge 1132 on the inferior component on a portion of the pedicle.

The length trial tool and/or head of the length trial tool may further comprise one or more reference markers 1180. The reference markers 1180 are desirably used when the length trial is inserted into the intervertebral disc space to help guide the surgeon and/or user on positioning of the length trial tool relative to the intervertebral disc space. The one or more reference markers may be confirmed radiographically to determine the length of insertion of the head of the length trial tool into the intervertebral disc space to achieve the desired length, desired tension and vertebral alignment. The one or more reference markers may comprise a radiopaque material or be radiopaque to allow visualization with an imaging equipment. Furthermore, the one or more reference markers may have its position confirmed radiographically to ensure that the head of the length trial tool is accurately positioned within the intervertebral disc space and to assess the anterior-to-posterior length of the intervertebral disc space relative to the length of the head of the length trial tool. The one or more reference markers may be disposed onto a portion of the head and/or shaft. In one embodiment, the one or more reference markers comprises a center of rotation (COR) marker 1134. The center of rotation marker 1134 may comprise a laser marking and/or an opening (see FIGS. 11E and 11F). The COR marker 1134 may be disposed to match or substantially match a distance of 40 percent anterior from the posterior, caudal endplate of the cranial vertebral body.

The length trial tool may comprise a shaft 1140 having a first end, a second end, a length, and/or a diameter. The diameter of the shaft may comprise a range of 5 mm to 8 mm; the diameter may further comprise a range of 6 mm to 8 mm; and/or the diameter may further comprise a range of 6 mm to 7 mm. The diameter of the shaft may be uniform along the longitudinal axis and/or length of the shaft. The shaft may comprise a shape, the shape may be cylindrical or generally cylindrical. The length of the shaft may comprise a range of 130 mm to 150 mm; the length of the shaft may comprise a range of 135 mm to 145 mm; and/or the length of the shaft may comprise a range of 140 mm to 145 mm. The head is disposed at a first end, and the connector disposed at a second end. The junction of first end of the shaft and head will desirably form an atraumatic shoulder that abuts or contacts against tissue or bone when the head is inserted between the two adjacent vertebral bodies. The shaft may be solid or hollow.

The length trial tool may comprise a connector, which may include a male and/or a female connector. The connectors may be configured to engage with a Hudson connection, an Association for Osteosynthesis (AO) connection and/or a Trinkle connection. The connection mechanisms comprise quick-connect and quick-disconnect. The connector may be disposed within and/or coupled to a manual handle and/or a powered handle or handpiece.

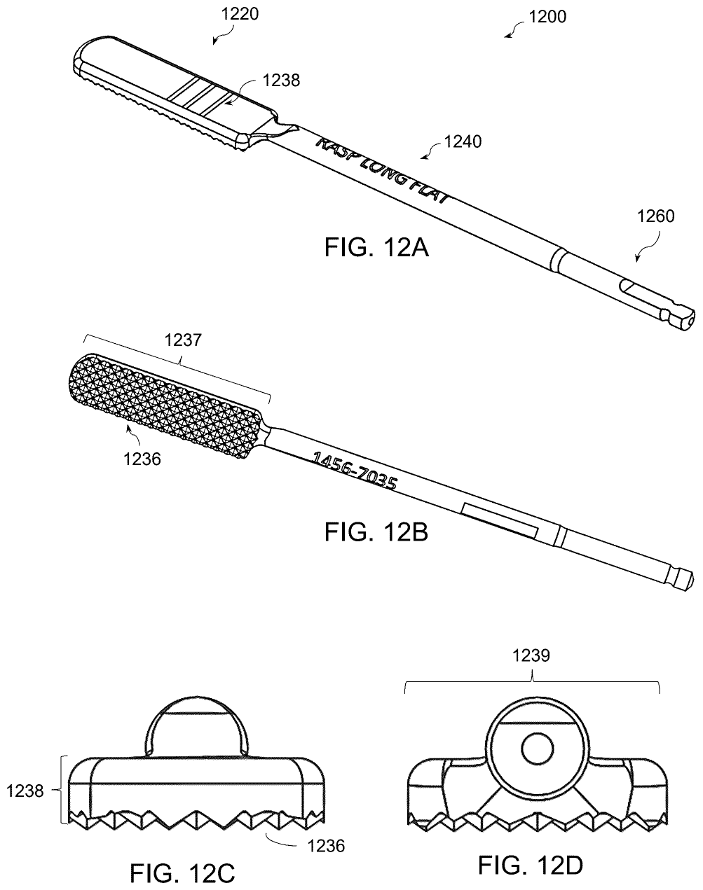

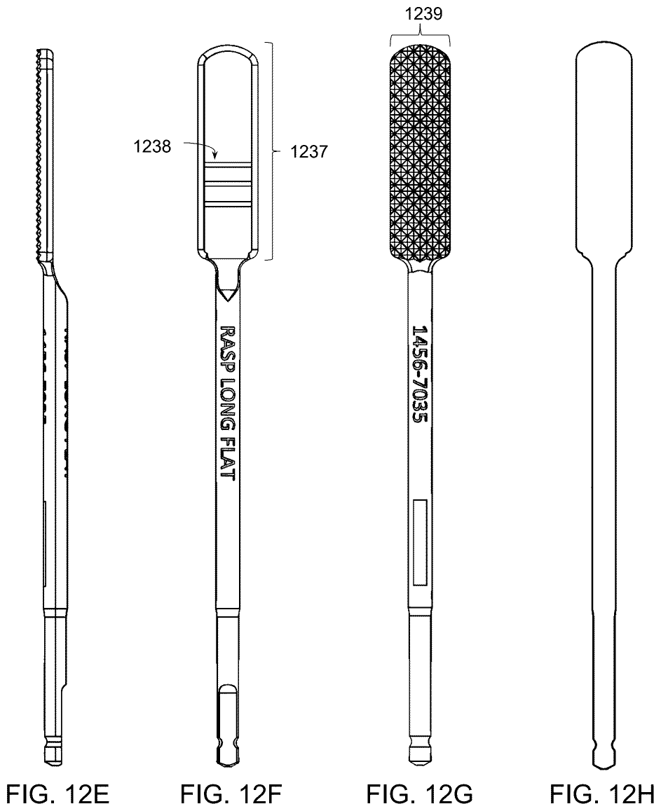

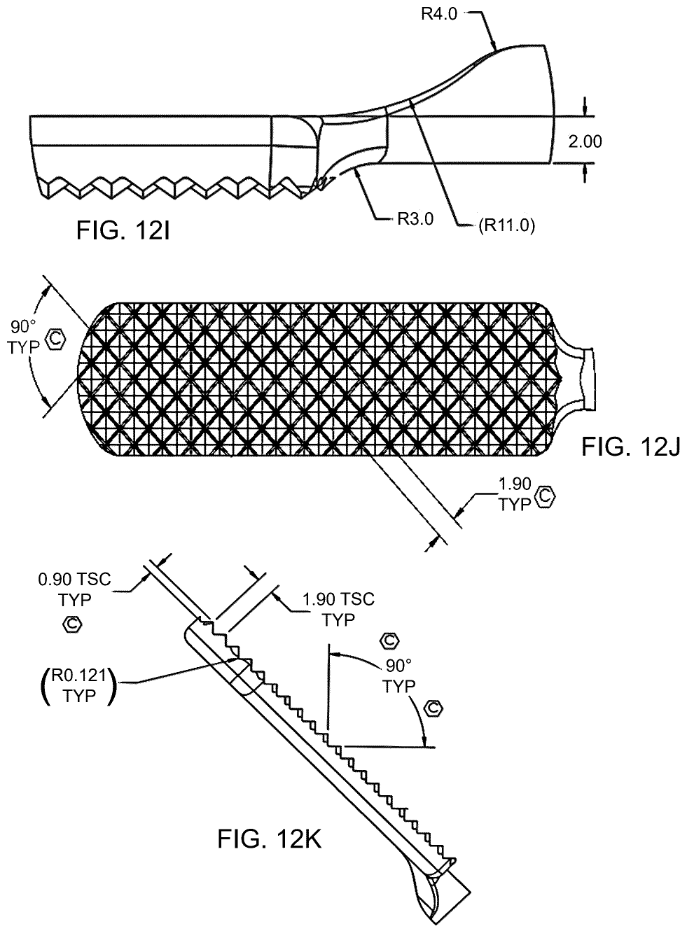

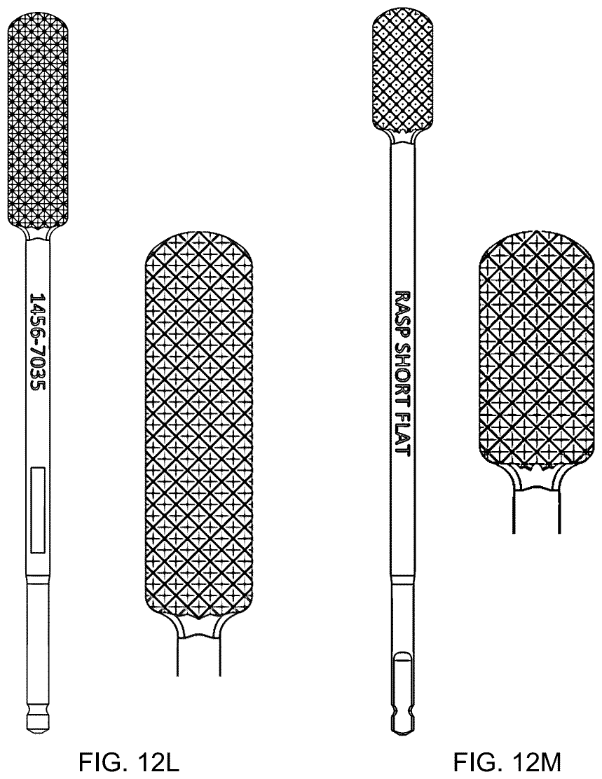

FIGS. 12A-12K depicts different plan views and a cross-sectional view of embodiment of a rasp tool 1200. The rasp tool 1200 can comprise a coarse file or similar surface that is used to sculpt bone, cartilage or other hard tissue in surgical procedures by trimming, reshaping, contouring, and/or forming surfaces. The rasp tool may comprise one or more sizes, including short and/or long rasp tools. The rasp tool comprises a head, tip or body 1220, a shaft 1240, and a connector 1260. FIGS. 12L-12M depicts a front view of the various available sizes of the rasp tool of FIGS. 12A-12K.

The head of the rasp tool comprises a first end 1222, a second end 1224, a width 1226, a height 1228 and a length 1230. The head of the rasp tool comprises a first surface 1232 and a second surface 1234. An abrasive surface 1236 can be disposed onto a portion of the first surface and/or a portion of the second surface. An abrasive surface 1236 can be disposed onto a portion of length of the first surface and/or a portion of the length of the second surface. The abrasive surface comprises one or more patterns, contours, and protrusions. In one embodiment, the one or more protrusions comprise a plurality of sharp teeth, the plurality sharp teeth project from a first and/or second surface of the head. The one or more protrusions may further comprise course or fine protrusions. The plurality sharp teeth are capable of cutting, scraping away and/or other removing a particular material from a surface. The one or more protrusions are disposed onto a first and/or second surface of the head in one or more different orientations, such that the orientations allow removal of material in one or more directions (e.g., forward direction, backward, and side directions).

The one or more protrusions may comprise different cross-sectional shapes or configurations, the cross-sectional shapes or configurations may include polygonal or rounded cross-sectional shapes. The polygonal cross-sectional shapes comprise triangular, square, heptagonal, hexagonal, and/or octagonal. The rounded cross-sectional shapes comprise circular, elliptical, and/or oval. The one or more protrusions may be disposed in one or more rows, each of the rows may be spaced apart. Each of the rows may be disposed parallel to each other. Each of the rows may be disposed parallel and orthogonal to each other. In another embodiment, the head of the rasp tool comprises a first one or more protrusions and a second one or more protrusions, the first one or more protrusions are arranged in a first one or more rows that are spaced apart and parallel to each other in a first orientation, the second one or more protrusions are spaced apart and parallel to each other in a second orientation, the first orientation is different than the second orientation. The second orientation is orthogonal to the first orientation.

The length 1237 of the head of the rasp tool may comprise a range of 25 to 50 mm; a range of 25-45 mm; and/or a range of 25-35 mm. The height or depth 1238 of the head may comprise sequential increasing height or depth, including starting at least 3 mm or greater with 1 mm increments. Alternatively, the height or depth of the head of the rasp tool may be the same for different sizes of rasp tool. The height or depth of the head comprises 2 to 5 mm; the height or depth further comprises 3 to 4 mm. The height or depth of the head of the rasp tool comprises at least a portion of uniform height along the longitudinal axis of the head and/or at least a portion of a non-uniform height along the longitudinal axis of the head. The height or depth of the head of the rasp tool comprise at least a portion of a uniform height along the length of the head and/or at least a portion of a non-uniform height along the length of the head. The width 1239 of the head may match or substantially match the width of the spinal implant. The width of each of the heads may be not change as the height or depth changes. The width may comprise a range of 10 to 15 mm; and/or the width may comprise a range of 10 to 13 mm.

The rasp tool and/or head of the rasp tool may further comprise one or more reference markers 1238. The reference markers 1238 are used when the rasp tool is inserted into the intervertebral disc space to help the user and/or surgeon prepare the bony and/or endplate surfaces of the superior or upper and/or inferior or lower vertebral body to the proper depth and/or orientation. The one or more reference markers 1236 may comprise a radiopaque material or be radiopaque to allow visualization with an imaging equipment. The one or more reference markers may comprise a laser marking, a ring, and/or an opening. The one or more reference markers may be disposed onto a portion of the head and/or the shaft.

The rasp tool may comprise a shaft 1240. The shaft comprises a first end, a second end, a length, and/or a diameter. The diameter of the shaft may comprise a range of 5 mm to 8 mm; the diameter may further comprise a range of 6 mm to 8 mm; and/or the diameter may further comprise a range of 6 mm to 7 mm. The diameter of the shaft may be uniform along the longitudinal axis and/or length of the shaft. The shaft may comprise a shape, the shape may be cylindrical or generally cylindrical. The length of the shaft may comprise a range of 60 mm to 100 mm; the length of the shaft may comprise a range of 75 mm to 100 mm; and/or the length of the shaft may comprise a range of 75 mm to 95 mm. The head is disposed at a first end, and the connector disposed at a second end. The junction of first end of the shaft and head forms an atraumatic shoulder that abuts or contacts against tissue or bone when the head is inserted between the two adjacent vertebral bodies. The shaft may be solid or hollow.

The rasp tool may comprise a connector 1260. The connector may include a male and/or a female connector. The connector is disposed at the second end of the shaft. The connectors may be configured to engage with a Hudson connection, an Association for Osteosynthesis (AO) connection and/or a Trinkle connection. The connection mechanisms comprise quick-connect and quick-disconnect. The connector may be disposed within and/or coupled to a manual handle and/or a powered handle or handpiece.

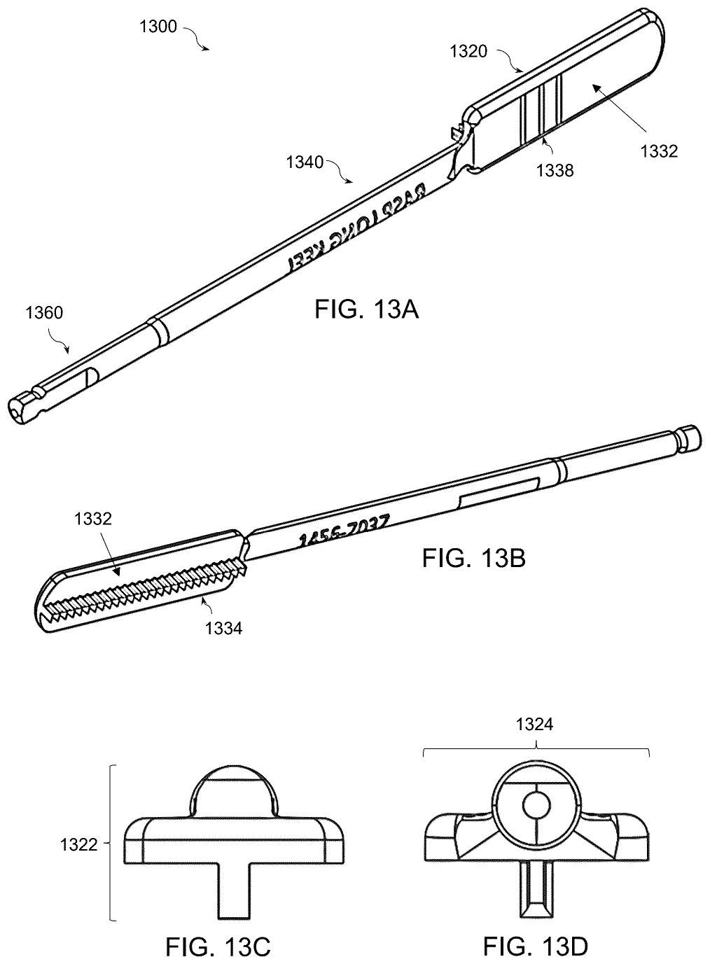

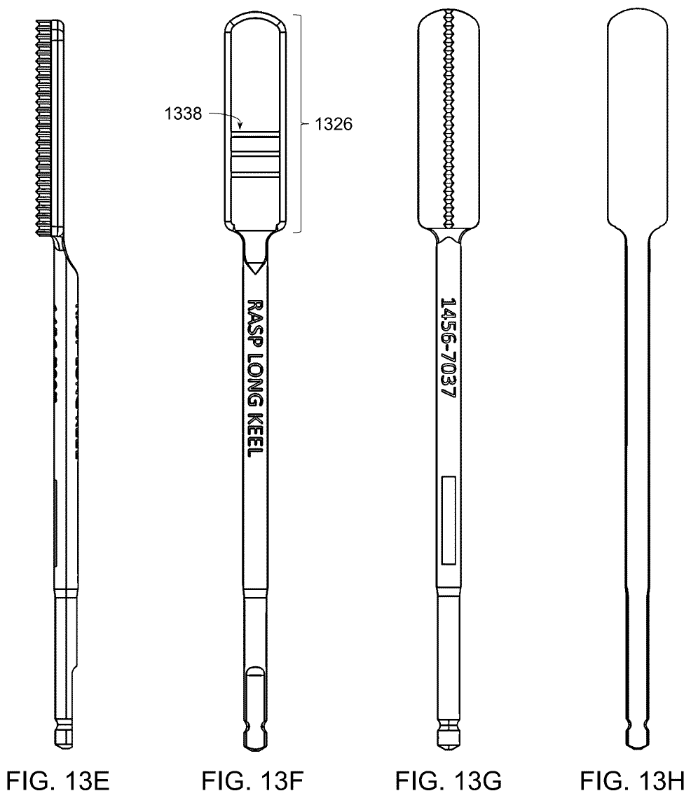

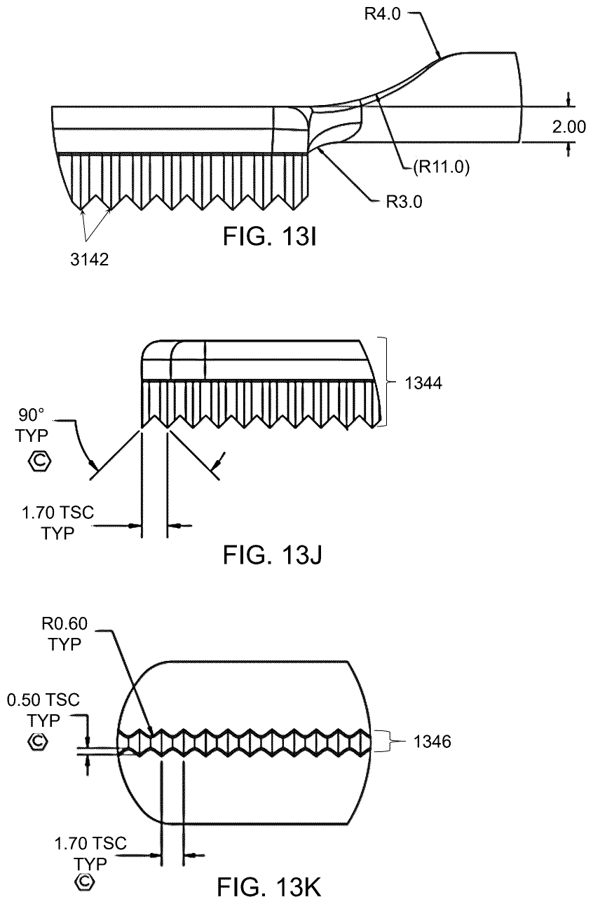

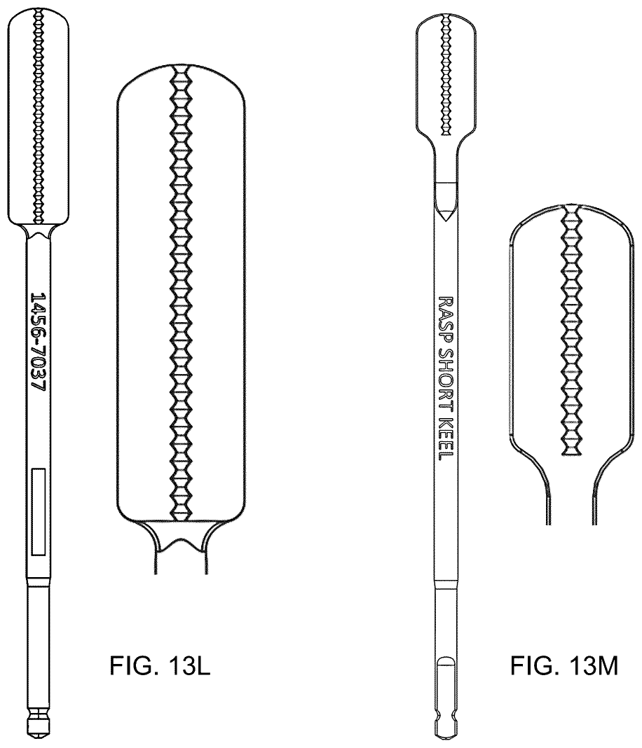

FIGS. 13A-13K depicts different plan views and a cross-sectional view of embodiment of a keel rasping or cutting tool 1300. The keel cutting tool 1300 is an instrument used to create one or more slots, channels or cutouts from bone, cartilage or other hard tissue in surgical procedures that are sized and configured to receive a portion of the keels of the one or more spinal implants. The keel cutting tool may comprise one or more sizes, and desirably comprises a head, tip or body 1320, a shaft 1340 and a connector 1360. The one or more sizes comprises a short and/or long keel cutting tool. FIGS. 13L-13M depicts a front view of the various available sizes of the keel cutting tool of FIGS. 13A-13K.

The head of the keel cutting tool comprises a first end, a second end, a width 1324, a height 1322 and a length 1326. The head of the keel cutting tool comprises a first surface 1332 and a second surface 1330. An abrasive surface or surface feature 1334 can be disposed onto a portion of the first surface and/or a portion of the second surface. An abrasive surface can be disposed onto a portion of length of the first surface and/or a portion of the length of the second surface. The abrasive surface comprises one or more textures and/or protrusions. The one or more textures and/or protrusions may further comprise contours and/or patterns. In one embodiment, the abrasive surface comprises one or more protrusions, the one or more protrusions comprise a plurality of sharp teeth 3142, the one or more protrusions project from a first and/or second surface of the head. The one or more protrusions may further comprise course or fine protrusions. The one or more protrusions and/or plurality sharp teeth are capable of cutting, scraping away and/or other removing a particular material from a surface. The one or more protrusions are disposed onto a first and/or second surface of the head in one or more different orientations, such that the orientations allow removal of material in one or more directions (e.g., forward direction, backward, and side directions).

The one or more protrusions may comprise different cross-sectional shapes or configurations, the cross-sectional shapes or configurations may include polygonal or rounded cross-sectional shapes. The polygonal cross-sectional shapes comprise triangular, square, heptagonal, hexagonal, and/or octagonal. The rounded cross-sectional shapes comprise circular, elliptical, and/or oval. The one or more protrusions may be disposed in one or more rows. The one or more protrusions may be disposed in one or more rows, each of the rows may be spaced apart. Each of the rows may be disposed parallel to each other. Each of the rows may be disposed parallel and orthogonal to each other. In another embodiment, the head of the keel rasp tool comprises a first one or more protrusions and a second one or more protrusions, the first one or more protrusions are arranged in a first one or more rows that are spaced apart and parallel to each other in a first orientation, the second one or more protrusions are spaced apart and parallel to each other in a second orientation, the first orientation is different than the second orientation. The second orientation is orthogonal to the first orientation.

The one or more protrusions comprises a height 1344 and/or a width 1346. The protrusion height may comprise a range of 2.5 mm to 3.5 mm; the range may comprise 3.0 mm to 3.5 mm; and/or the height may comprise a range of 3.0 mm to 3.3 mm. Alternatively, the protrusion height may match or substantially match the keel height of a spinal implant. The protrusion width may comprise a range of 1.0 mm to 2 mm; the width may comprise a range of 1.5 mm to 2 mm; and/or the width may comprise a range of 1.6 mm to 1.9 mm. Alternatively, the protrusion width may match or substantially match the keel width of the spinal implant. The protrusion width may comprise a larger width than that the keel width of the spinal implant.

The length of the head of the keel cutting tool comprises a range of 25 to 50 mm; a range of 25-45 mm; and/or a range of 35-45 mm. The height or depth of the head of the keel cutting tool comprises sequential increasing height or depth, including starting at least 3 mm or greater with 1 mm increments. Alternatively, the height or depth of the head of the keel cutting tool may be the same for different sizes of rasp tool. The height or depth of the head comprises 3 to 7 mm; and/or 4 to 6 mm. The height or depth of the head of the keel cutting tool comprises at least a portion of uniform height along the longitudinal axis of the head and/or at least a portion of a non-uniform height along the longitudinal axis of the head. The height or depth of the head of the keel cutting tool comprise at least a portion of a uniform height along the length of the head and/or at least a portion of a non-uniform height along the length of the head. The width of the head of the keel cutting may match or substantially match the width of the spinal implant. The width of each of the heads of the keel cutting tools may be not change as the height or depth changes. The width may comprise a range of 10 to 15 mm; and/or the width may comprise a range of 11 to 13 mm.

The keel cutting tool and/or head of the keel cutting tool may further comprise one or more reference markers 1338. The reference markers are used when the keel cutting tool is inserted into the intervertebral disc space to help the user and/or surgeon prepare at least one keel channel onto prepared and/or resected endplate surfaces of the superior or upper and/or inferior or lower vertebral body to the proper depth and/or orientation. The one or more reference markers may comprise a radiopaque material or be radiopaque to allow visualization with an imaging equipment. The one or more reference markers may comprise a laser marking, a ring, and/or an opening. The one or more reference markers may be disposed onto a portion of the head and/or the shaft. The one or more reference markers may comprise depth markings. The depth markings determine the depth of the keel rasp tool with relative to the anterior-to-posterior width of a vertebral body.

The keel cutting tool may comprise a shaft 1340 having a first end, a second end, a length, and/or a diameter. The diameter of the shaft may comprise a range of 5 mm to 8 mm; a range of 6 mm to 8 mm; and/or a range of 6 mm to 7 mm. The diameter of the shaft may be uniform along the longitudinal axis and/or length of the shaft. The shaft may comprise a shape, the shape may be cylindrical or generally cylindrical. The length of the shaft may comprise a range of 60 mm to 100 mm; a range of 75 mm to 100 mm; and/or a range of 75 mm to 90 mm. The head is disposed at a first end, and the connector disposed at a second end. The junction of first end of the shaft and head forms an atraumatic shoulder that abuts or contacts against tissue or bone when the head is inserted between the two adjacent vertebral bodies. The shaft may be solid or hollow.

The keel cutting tool may comprise a connector, such as a male and/or a female connector. The connector is disposed at the second end of the shaft. The connectors may be configured to engage with a Hudson connection, an Association for Osteosynthesis (AO) connection and/or a Trinkle connection. The connection mechanisms comprise quick-connect and quick-disconnect. The connector may be disposed within and/or coupled to a manual handle and/or a powered handle or handpiece.

In one embodiment, the distractor, the height trial, the length trial, the rasp tool, and/or the keel cutting can comprise a first material such as a polymer, a metal, a ceramic and/or various combinations thereof. The polymer may include a thermoset or thermoplastic. The metal may include stainless steel, a stainless steel alloy, titanium, titanium alloy, cobalt chrome, cobalt chrome alloy, nitinol, and/or tantalum. The distractor, the height trial, the length trial, the rasp tool, and/or the keel rasp may further comprise electropolishing and/or passivation.

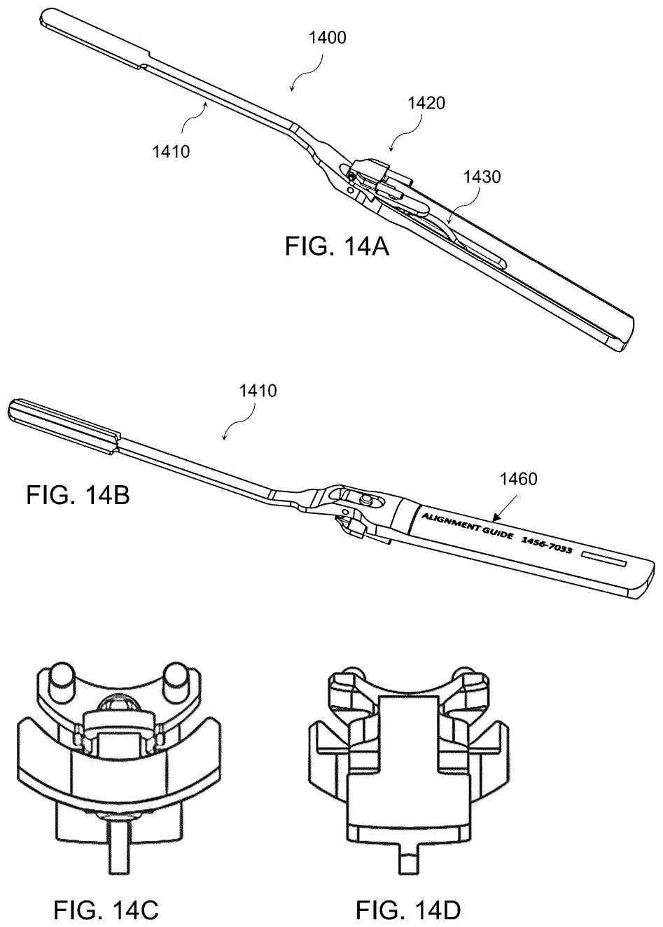

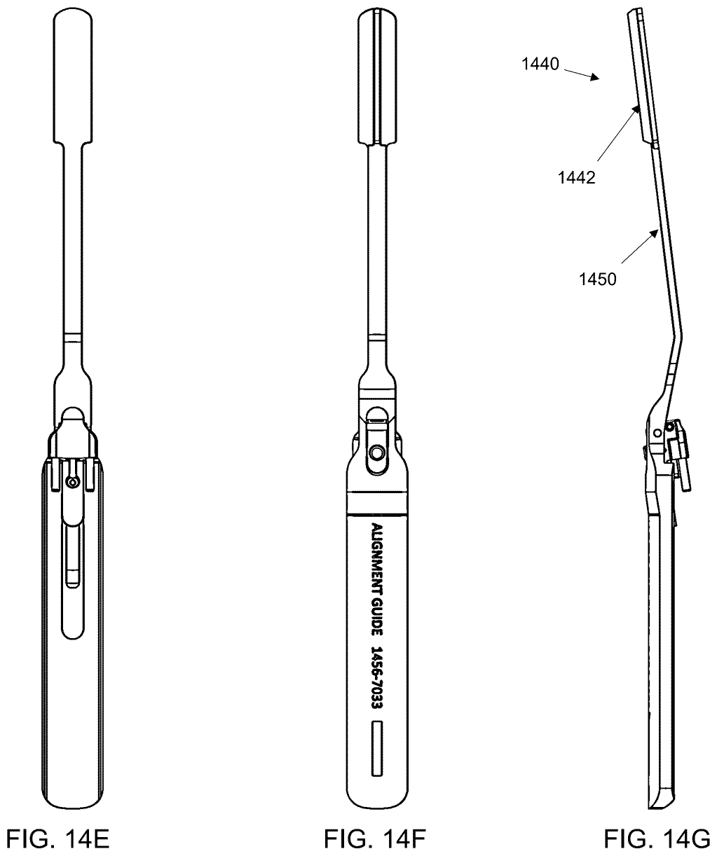

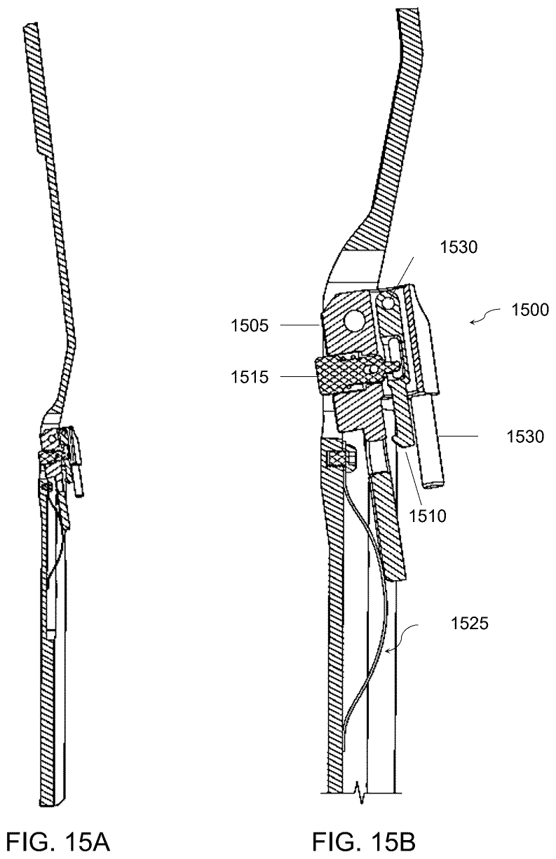

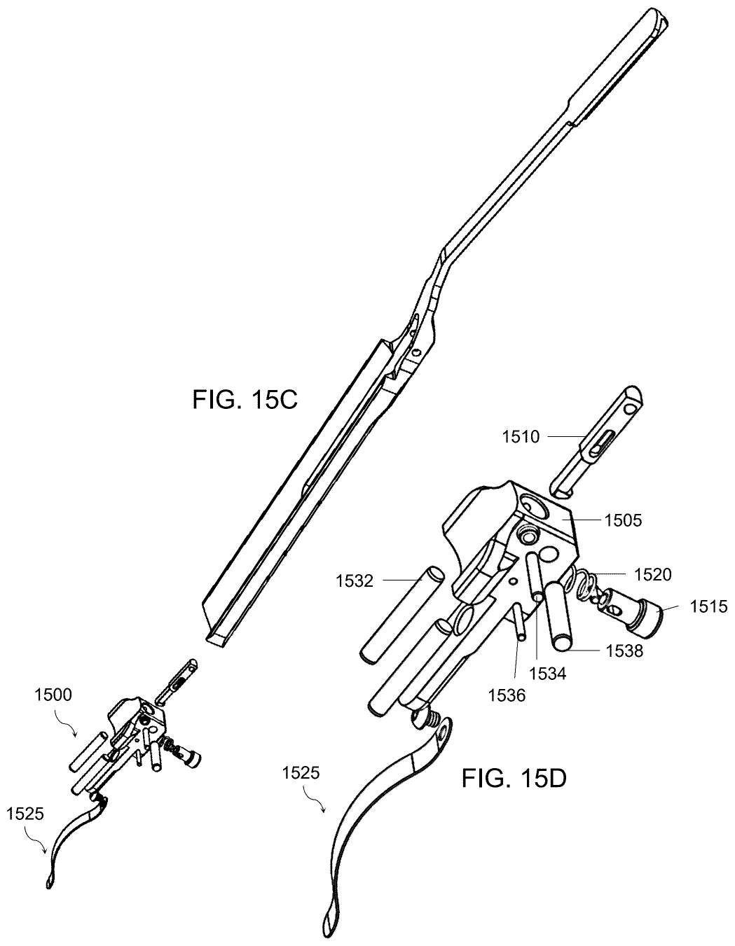

FIGS. 14A-14G depicts different plan views and cross-sectional views of one exemplary embodiment of a keel alignment guide assembly 1400 and FIGS. 15A-15B depict cross-sectional views of the keel alignment guide assembly, and FIGS. 115C and 15D depict exploded views of the keel alignment guide assembly. The keel alignment guide assembly 1400 is an instrument used to help align a first one or more slots, channels or cutouts from bone, cartilage or other hard tissue into a first vertebral body relative to a second one or more slots, channels or cutouts from bone, cartilage or other hard tissue into a second vertebral body in surgical procedures that are sized and configured to receive a portion of the keels of the one or more spinal implants. The first vertebral body and/or the second vertebral body may comprise the inferior or superior vertebral body. The alignment guide assembly 1400 may comprise one or more sizes to accommodate the different keel rasp tool sizes. The one or more sizes of the keel rasp tool comprises a short and/or long keel rasp tool.

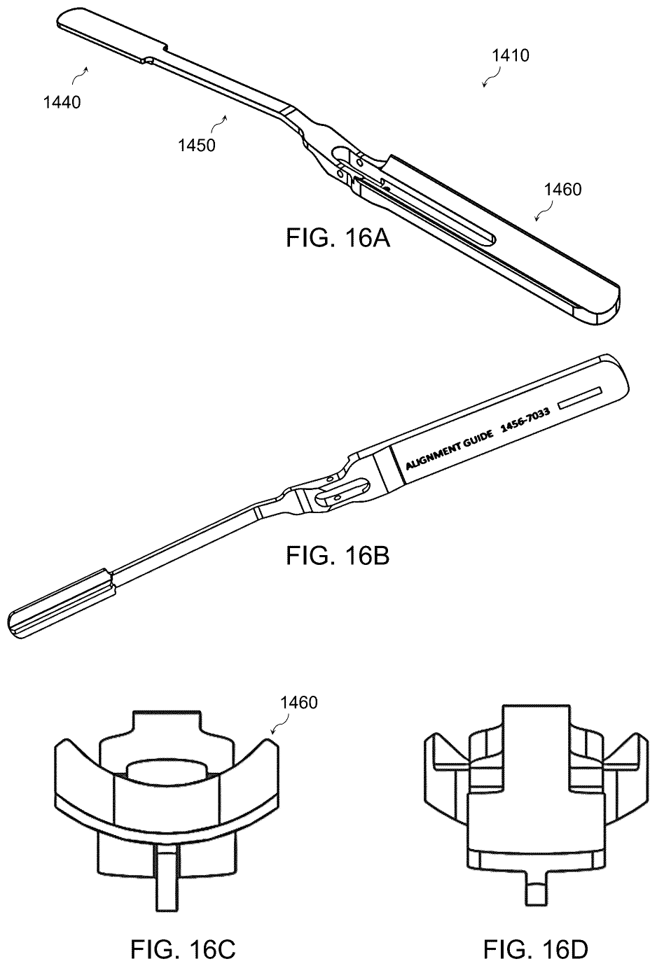

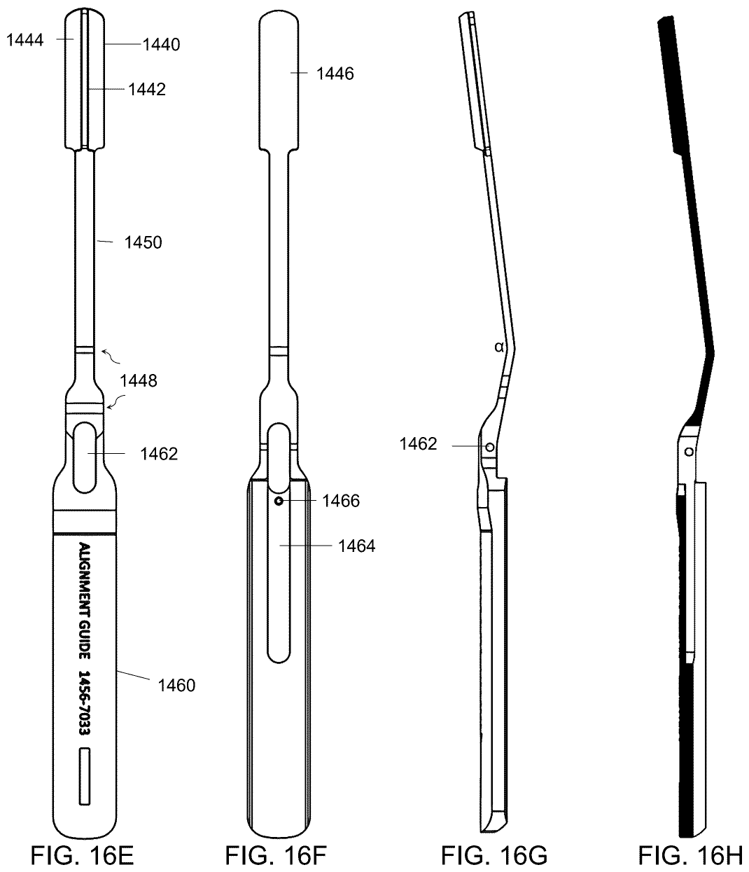

The keel alignment guide assembly comprises a guide arm 1410, a locking mechanism 1420 and an optional leaf spring 1430. The guide arm 1410 comprises a guide head 1440, a guide shaft 1450 and a handgrip or guide handgrip 1460 as shown in FIGS. 16A-16H. The guide arm further comprises a first end and a second end. The guide head 1440 having a first end, a second end, and a width, a height and a length. The guide head assembly further comprises a keel or protrusion 1442, a first surface 1444 and a second surface 1446. The guide head disposed at a first end of the guide arm. The one or more keels or one or more protrusions project from a first and/or second surface of the guide head. In another embodiment, the one or more keels may project or extend from a first surface. The one or more keels may extend orthogonal or oblique from the first surface. The one or more keels or protrusions further comprises a first side surface and a second side surface. The first and/or second side surface may comprise a flat and smooth surface. The first and/or second side surface may comprise a flat and abrasive surface. At least a portion of the first and/or second side surface of the one or more protrusions or keels may contact or engage with a keel channel disposed onto a first or second vertebral body.

The one or more protrusions or keels may comprise different cross-sectional shapes or configurations, the cross-sectional shapes or configurations may include polygonal or rounded cross-sectional shapes. The polygonal cross-sectional shapes comprise triangular, rectangular, square, heptagonal, hexagonal, and/or octagonal. The rounded cross-sectional shapes comprise circular, elliptical, and/or oval. The one or more protrusions or keels may be disposed in one or more rows. The one or more protrusions or keels may be disposed in one or more rows, each of the rows may be spaced apart. Each of the rows may be disposed parallel to each other. Each of the rows may be disposed parallel and orthogonal to each other.

The one or more protrusions or keels comprises a length, height and/or a width. The protrusion or keel height may comprise a range of 2 mm to 4.0 mm; the range may comprise 2.5 mm to 3.5 mm; and/or the height may comprise a range of 3.0 mm to 3.3 mm. Alternatively, the protrusion height may match or substantially match the keel height of a spinal implant. The protrusion height may match or substantially match the keel channel height of a first or second vertebral body. The protrusion or keel width may comprise a range of 1.0 mm to 2 mm; the width may comprise a range of 1.5 mm to 2 mm; and/or the width may comprise a range of 1.7 mm to 1.9 mm. Alternatively, the protrusion or width may match or substantially match the keel width of the spinal implant. The protrusion width may be smaller than the keel channel width of a first or second vertebral body. The protrusion or keel width may comprise a larger width than that the keel width of the spinal implant. The protrusion or keel length may comprise a length of 35 mm to 50 mm; the length may comprise 40 mm to 48 mm; and/or the length may comprise 44 mm to 46 mm. The protrusion or keel length may extend along at least a portion of the guide head. The protrusion or keel length may extend along the entire length of the guide head. The protrusion or keel length may match or substantially match the guide head length.

The guide head length comprises a length of 35 mm to 50 mm; the length may comprise 40 mm to 48 mm; and/or the length may comprise 44 mm to 46 mm. The height or depth of the guide head comprises sequential increasing height or depth, including starting at least 1.5 mm or greater with 1 mm increments. Alternatively, the guide head height or depth may be the same for different sizes of alignment guide assemblies. The guide head height or depth comprises 1.0 to 3 mm; the height or depth further comprises 1.25 to 2.5 mm; and/or the guide head depth or height may comprise 1.5 to 1.75 mm. The guide head height or depth of comprises at least a portion of uniform height along the longitudinal axis of the head and/or at least a portion of a non-uniform height along the longitudinal axis of the head. The guide head height or depth comprises at least a portion of a uniform height along the length of the head and/or at least a portion of a non-uniform height along the length of the head. The guide head width may match or substantially match the width of the spinal implant. The guide head width may match or substantially match the prepared and/or resected surface width. The guide head width may be not change as the height or depth changes. The width may comprise a range of 10 to 15 mm; and/or the width may comprise a range of 11 to 13 mm. At least a portion of the first contact surface and/or the second contact surface of the guide head contacts or engages a portion of the prepared or resected endplate surface of a vertebral body.

The guide arm may comprise a guide shaft. The guide shaft comprises a first end, a second end, a length, and/or a width. The guide shaft width may comprise a range of 5 mm to 8 mm; the guide shaft width may further comprise a range of 5.5 mm to 7 mm; and/or the diameter may further comprise a range of 6 mm to 7 mm. At least a portion of the guide shaft width may be uniform along the longitudinal axis and/or length of the shaft. The guide shaft length may comprise a range of 90 mm to 120 mm; the length of the shaft may comprise a range of 100 mm to 110 mm; and/or the guide shaft length may comprise a range of 105 mm to 107 mm. The guide head is disposed at a first end of the guide shaft or guide arm. The shaft may be solid or hollow. The guide shaft may comprise a non-planar or non-straight shape along the length of the shaft. The guide shaft may comprise an at least a portion of the guide shaft length including one or more angles, a. The shaft is disposed between the guide head and the guide handgrip.

The guide arm, the guide head, guide shaft and/or guide handgrip may further comprise one or more reference markers 1448. The reference markers are used when the keel alignment guide is inserted into the intervertebral disc space to help the user and/or surgeon align at least two keel channels onto prepared and/or resected endplate surfaces of the superior or upper and/or inferior or lower vertebral body to the proper depth and/or orientation. The one or more reference markers may comprise a radiopaque material or be radiopaque to allow visualization with an imaging equipment. The one or more reference markers may comprise a laser marking, a ring, and/or an opening. The one or more reference markers may be disposed onto a portion of the head, the shaft and/or handgrips. The one or more reference markers may comprise depth markings. The depth markings determine the depth of the guide arm with relative to the anterior-to-posterior width of a vertebral body.

The guide arm further comprises a handgrip. The handgrip comprises a length, a width and one or more openings. The guide handgrip length comprises a length of 115 mm to 140 mm; the length comprises 120 mm to 130 mm; and/or the length comprises 124 mm to 126 mm. The width of the guide handgrip comprises 10 mm to 30 mm; the guide handgrip width may comprise 15 mm to 25 mm; and/or the guide handgrip width may comprise 19 mm to 21 mm. The guide handgrip further comprises a one or more openings 1462. In another embodiment, the guide handgrip comprises a first one or more openings or a slot and a second one or more openings. At least a portion of the first one or more openings extends from the first surface through the second surface of the guide handgrip. The first one or more openings is sized and configured to receive at least a portion of the connection mechanism or a portion of the base of the connection mechanism. The second one or more openings extends from a first side surface through a second side surface. The second one or more openings are sized and configured to receive one or more dowel pins for motion and/or pivotal motion. The first surface and/or second surface comprises a shape. The shape is concave or hemispherical. The shape may match or substantially match the diameter or width of a handle. The handle may comprise a manual handle and/or powered handpiece. The first surface contacts or engages at least a portion of the outer diameter or width of the handle.

The guide handgrip may further comprise a recess 1464. The recess may include a counterbore 1466. The counterbore may comprise threads. The counterbore may be sized and configured to receive a fastener. The recess comprises a width and a length. The recess is sized and configured to receive at least a portion of a leaf spring. At least a portion of leaf spring may be coupled to a portion of the guide arm or guide handgrip. The leaf spring comprises a flat strip of material that is loaded as cantilevers or beams while the surgeon is compressing the guide arm toward the handle. Furthermore, while the compression is occurring, the leaf spring deflects to further provide an opposing force and is storing the potential or kinetic energy. Once the user ceases compression of the guide arm towards the handle, the energy is dissipated allowing the leaf spring to return to its original position forcing the guide arm to separate from the handle at an angle.

The keel alignment guide assembly may comprise a connection or locking mechanism 1500. FIGS. 15A-15B depicts cross-sectional views, and FIGS. 15D and 15D depict exploded views of one embodiment of the connection or locking mechanism 1500, including a base 1505, a latch 1510, a button 1515, a compression spring 1520, a leaf spring, 1525 and/or a plurality of dowels 1530. The plurality of dowels may comprise a different diameter or different sizes. The plurality of dowels may comprise a first dowel 1532, a second dowel 1534, a third dowel 1536, and a fourth dowel 1538.

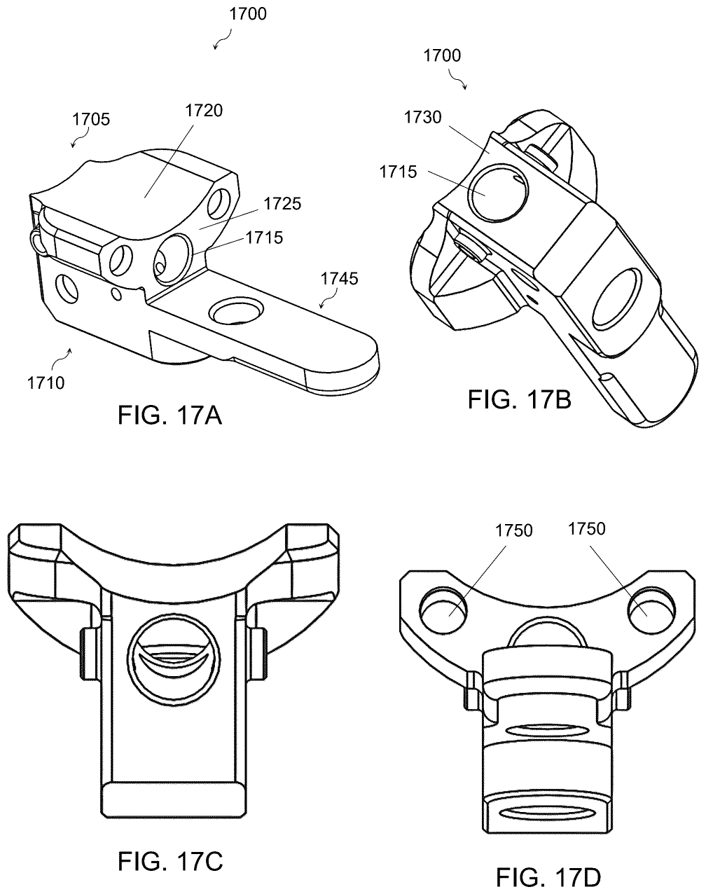

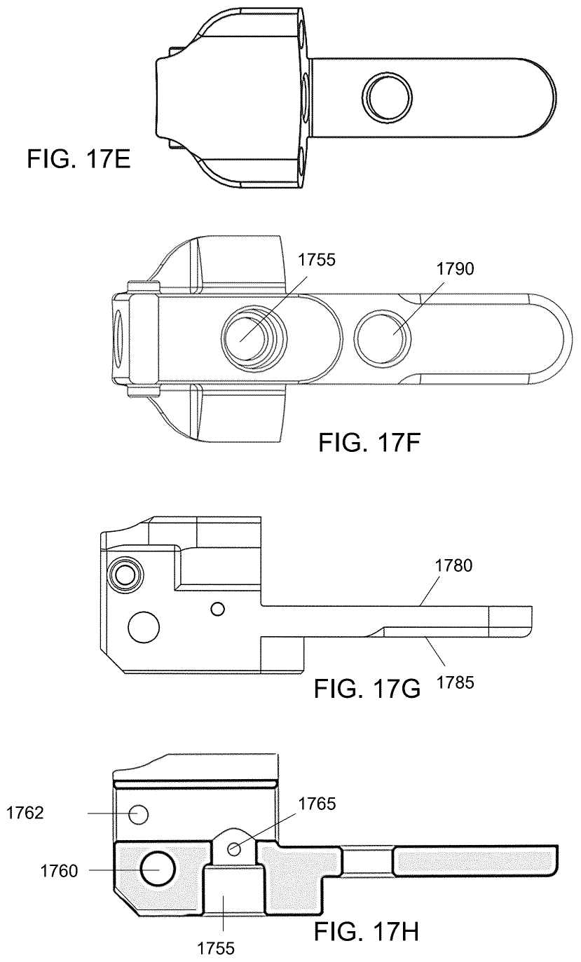

FIGS. 17A-17H depict various plan views and a cross-sectional view of one embodiment of a base 1700 of the connection or locking mechanism. The base comprises a top portion 1705 or first portion and a bottom portion 1710 or a second portion. The first portion or top portion of the base comprises a plurality of dowel pin openings and at least one latch opening 1715. The first portion or the top portion further comprises a first contact surface 1720. The plurality of dowel pin openings extend from the front surface 1725 through the back surface 1730. The plurality of dowel pin openings is desirably sized and configured to receive at least a portion of the at least one or more of the first dowel pins. Each of the plurality of dowel pin openings comprising an inner surface. The inner surface comprising a diameter and a length. The length of the inner surface of the dowel pin opening is smaller than the one or more first dowel pin length. The diameter of the inner surface of the dowel pin openings 1750 may be larger than the diameter of the one or more first dowel pins. At least a portion of the one or more first dowel pins may be disposed within the plurality of dowel pin openings for a press fit or friction fit. At least the remaining portion of the one or more first dowel pins extend outwardly, the at least of the remaining portion of the one or more first dowel pins may contact or engage with the handle to help facilitate coupling.

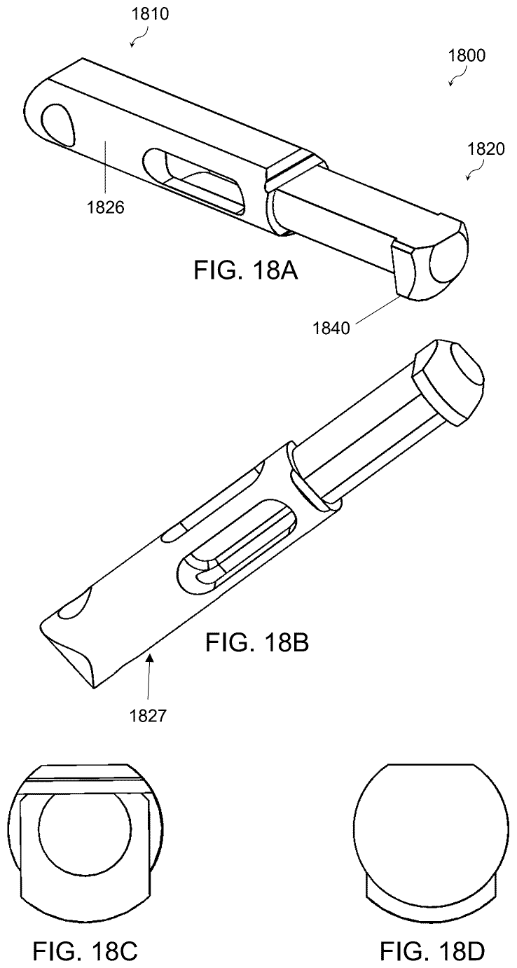

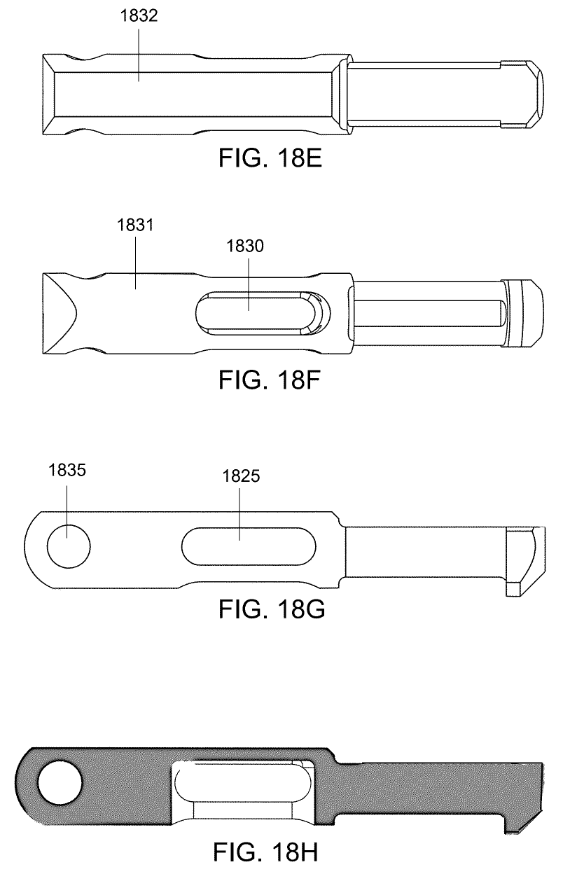

The first portion or top portion of the base comprises a latch opening. The latch opening is sized and configured to receive a portion of the latch (see FIG. 18A-18H) and/or the second end of the latch. The at least one latch opening comprises a latch inner surface. The inner surface comprising a diameter and a length. The length of the inner surface of the latch opening is smaller than the at least one latch length. The diameter of the inner surface of the at least one latch opening may be larger than the diameter of the latch. At least a portion of the at least one latch may be disposed within the at least one latch openings for a slip fit, press fit or friction fit. At least the remaining portion of the at least one latch extends outwardly, the at least of the remaining portion of the at least one latch may contact or engage with the handle to help facilitate coupling. Alternatively, at least a portion of the first portion of the latch may be disposed within the at least one latch openings and the second portion extends outwardly away from the at least one latch openings. The second portion of the at least one latch may contact or engage with the handle to facilitate coupling.

The first portion or top portion and/or second portion or bottom portion may further comprise a second dowel pin opening 1762. The second dowel pin opening is sized and configured to receive one or more second dowel pins. The second dowel pin opening is positioned transverse or substantially transverse to the at least one latch opening. The second dowel pin opening extends from a first side of the first or second portion through the second side of the first or second portion. The one or more second dowel pins is disposed into the second dowel pin opening and extends through the latch body or second end of the latch through-hole to secure or couple the latch to the base.

The second portion or the bottom portion of the base comprises an extension tab 1745, a button opening 1755, a third down pin opening 1760 and fourth dowel pin opening 1765. At least a portion of the connection mechanism is disposed into at least a portion of the first one or more openings or slot of the guide handgrip. Alternatively, at least a portion of the bottom or second portion is disposed into at least a portion of the first one or more openings or slot of the guide handgrip. The extension tab extends from the front surface of the base. The extension tab is flat and/or planar. The extension tab comprises a first surface 1780, a second surface 1785 and a fastener opening 1790. The fastener opening is larger than the head of the fastener. The fastener may be inserted through the fastener opening and allow a driver to secure the fastener to the leaf spring. The fastener opening on the extension tab is coaxially aligned with a fastener opening on the leaf spring. Accordingly, at least a portion of the second surface contacts or engages with the leaf spring. At least a portion of the first surface contacts or engages with the outer diameter of the handle. The extension tab facilitates or helps the compression of the leaf spring while the guide arm is squeezed or compressed towards the handle.

The bottom portion of the base comprises a button opening. The button opening on the bottom portion of the base extends from the bottom surface of the base towards a portion of the top surface. The button opening is sized and configured to receive a portion of the button. A fourth dowel pin opening is disposed transverse to the button opening. The fourth dowel pin opening extends from a first side through the second side of the base. The fourth dowel pin opening is disposed near or proximate to the second end of the button opening. The fourth dowel pin opening is sized and configured to receive a one or more fourth dowel pins. The one or more fourth dowel pins are disposed into the fourth dowel pin opening through the opening on a portion of the button and/or through the opening on the second end of the button. The one or more fourth dowels pins are used to secure and/or couple the button to the base.

The bottom portion of the base comprises a third dowel pin opening. The third dowel pin opening extends from a first side through a second side of the base. The third dowel pin opening is transverse to the button opening. The third dowel pin opening is parallel to the second and/or fourth dowel pin opening. The third dowel pin opening is sized and configured to receive a one or more third dowel pins. The one or more third dowel pins are inserted through the second one or more openings of the guide handgrip and into the third dowel pin opening of the bottom portion of the base to provide pivotal motion between the handle and the keel alignment guide assembly.

FIGS. 18A-18H depict various plan views and a cross-sectional view of one embodiment of a latch 1800. The latch comprises a first portion 1810 and a second portion 1820. The first portion includes a first slot 1825, a second slot 1830 and an opening or through-hole 1835. The first slot extends from a first side surface 1826 through the second side surface 1827. The first slot is sized and configured to receive a portion of the grasp tip of the button. The second slot is positioned transverse to the first slot. The second slot is ninety degrees apart from the first slot. The second slot extends from a bottom surface 1831 towards a portion of the top surface 1832. The second slot intersects with the first slot. The second slot is sized and configured to receive a portion of the button body. The first and/or the second slot comprises a length. The length is set to motion translation limit for distance. At least a portion of the grasp tip is disposed into a portion of the first slot to allow the button to slide, move or pivot relative to the latch while the guide arm is compressed or uncompressed towards the handle. At least a portion of the second portion of the latch may be disposed into the latch opening.

The second portion of the latch and/or the latch comprises a through-hole. The through-hole of the latch is sized and configured to receive a portion of the one or more second dowel pins. The through-hole of the latch is coaxial to the second dowel pin opening. The one or more second dowel pins are disposed through the second dowel pin opening of the base through the through-hole of the latch to couple the latch to the base. The through hole extends from a first side surface through to the second side surface.

The first portion of the latch and/or the latch comprises a hook 1840. The first portion of the latch extends away from the at least one latch opening. At least a portion of the hook extends over the fastener opening of the extension tab of the base. At least a portion of the hook may contact or engage a portion of the handle to couple the keel alignment guide assembly to the handle. Alternatively, at least a portion of the hook may contact or engage with a portion of the fastener opening to couple lock the keel alignment guide to the handle.

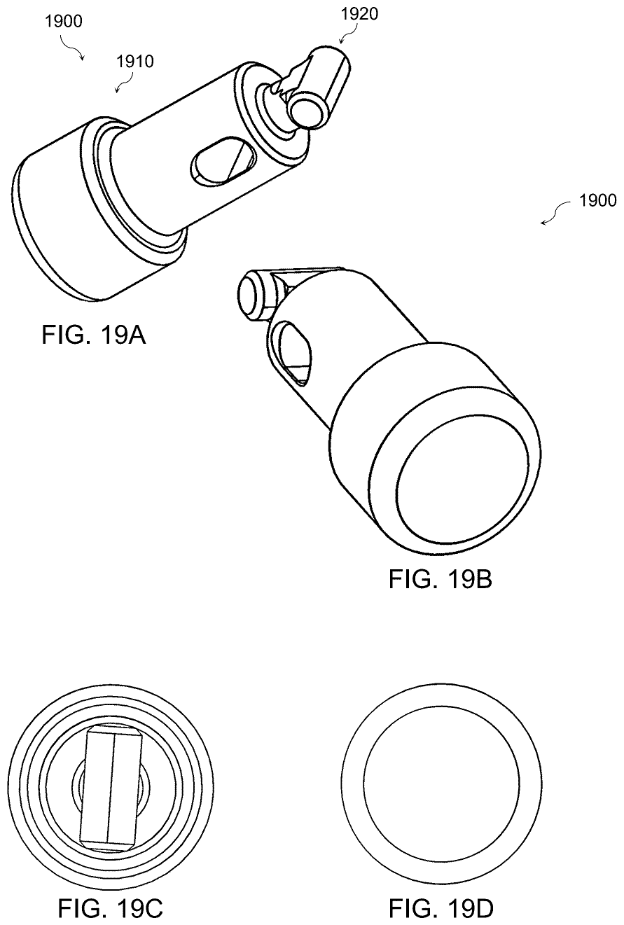

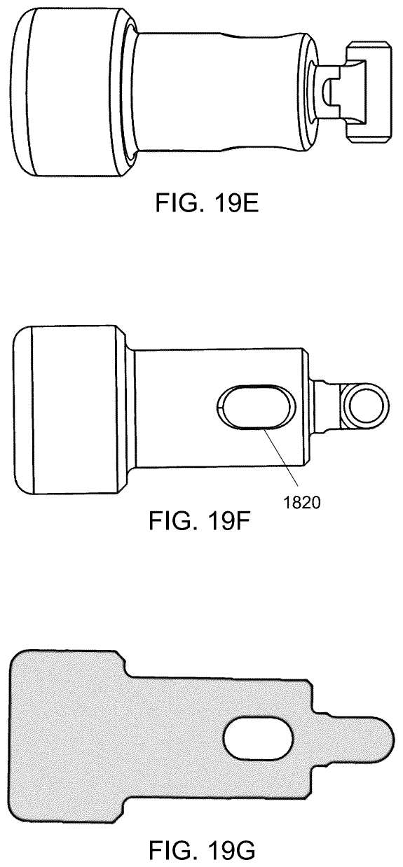

FIGS. 19A-19G depicts various plan views and across-sectional view of one embodiment of a button. The button comprises a body 1910 and a grasping tip 1920. The body includes a first outer diameter and a second outer diameter. The first outer diameter is sized and configured to receive a portion of a compression spring. The second outer diameter is larger than the first outer diameter. At least a portion of the compression spring contacts or abuts against a surface of the second outer diameter. At least a portion of the button is disposed into the at least one button opening on the base or the bottom portion of the base. At least the first outer diameter of the button is disposed into a portion of the at least one button opening of the base and/or the at least one button opening of the bottom portion of the base. The compression spring and the button assembly is disposed into the at least one button opening of the base and/or the at least one button opening of the bottom portion of the base. While the grasping tip of the button is disposed within the first slot of the latch, the compression spring, at rest, provides a downward force onto the button—the button is not activated. Conversely, once the button is pressed or compressed, the second diameter of the button pushes or compresses the compression spring and the grasping tip lifts the latch upwardly to release the latch from the handle and/or the fastener opening of the extension tab of the base. Once the button is released, the compression spring returns to its original position, re-engaging or contacting a portion of the handle and/or the portion fastener opening of extension tab of the base.

At least a portion of the grasping tip is sized and configured to be disposed within a first slot of the latch. The grasping tip is “T” shaped. Also, the body of the button comprises a through-hole 1820. The through-hole is sized and configured to receive a portion of the one or more fourth dowel pins. The one or more fourth dowel pins are inserted through the fourth dowel pin opening of the base and/or the bottom portion of the base and through the through-hole of the body of the button. The through-hole of the button or the body of the button is coaxially aligned with the one or more fourth dowel pin openings of the base or the bottom portion of the base.

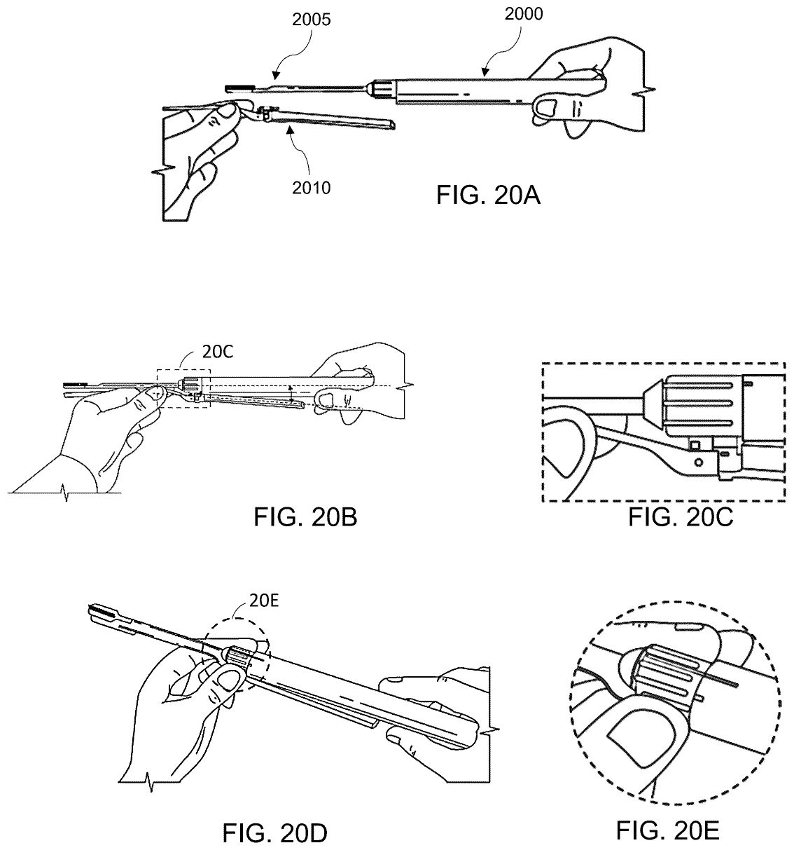

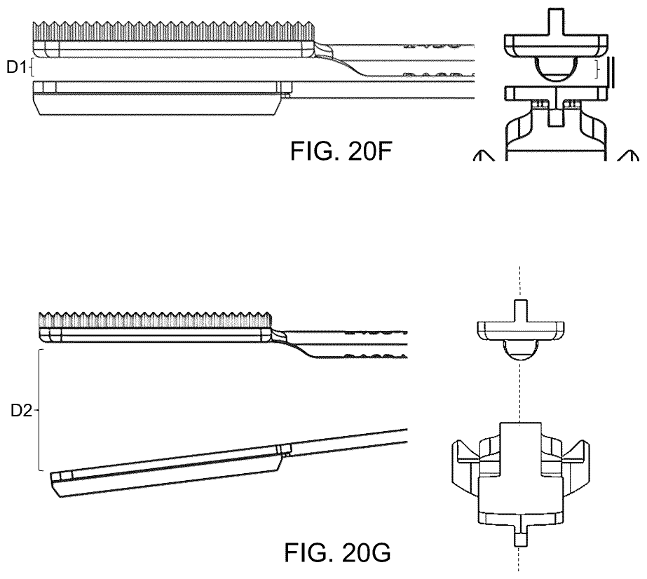

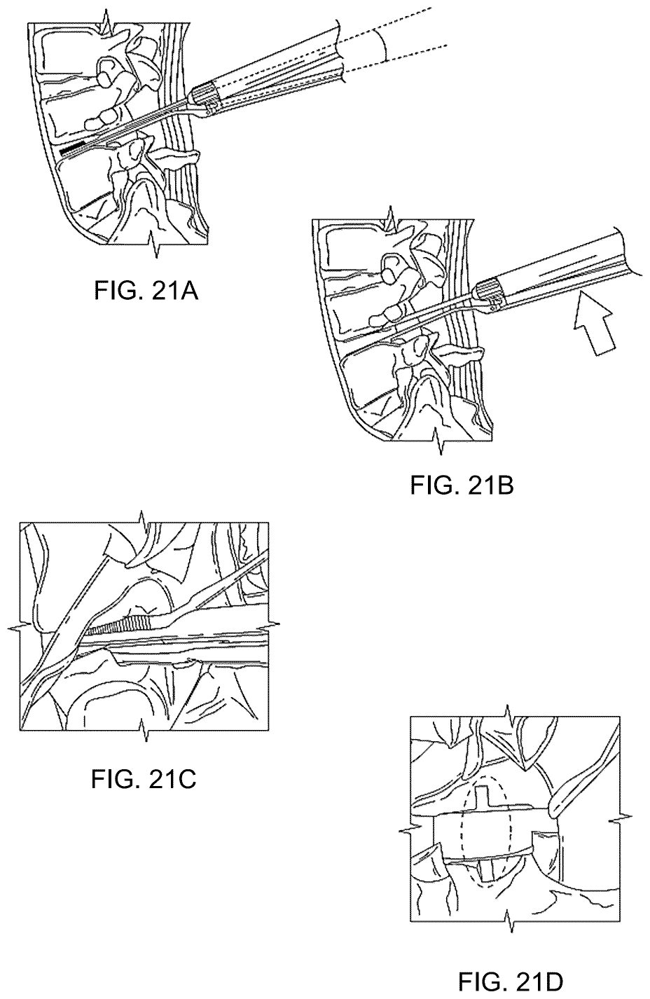

FIGS. 20A-20G and 21A-21D depicts various plan views of the assembly and operation of the keel alignment system. The keel alignment system comprises a handle or powered handpiece 2000; a keel rasp 2005; and a guide tool or alignment guide 2010. In one embodiment, the keel alignment system comprises a handle; a fixed keel rasp disposed substantially in a first orientation or plane, the fixed keel rasp comprising a keel rasp head, the keel rasp head comprising a first one or more protrusions, the first one or more protrusions extending towards the superior direction; and a movable guide tool assembly disposed substantially in a second orientation or plane, the movable guide arm removably coupled to the handle, the movable guide tool assembly comprising a guide arm, the guide arm including a guide head and a hand grip, the guide head comprising a second one or more protrusions, the second one or more protrusions extending towards the inferior direction, the movable guide tool assembly is angled and moves relative to the fixed keel rasp from the second orientation towards the first orientation until a portion of the hand grip contacts or engages at least a portion of the handle when compressing or squeezing a portion of hand grip of the movable guide tool assembly to create a spacing between the guide head and the keel rasp head.

The handle comprises a powered handle or a manual handle. The first one or more protrusions of the fixed keel rasp is coaxially aligned with the second one or more protrusions of the movable guide tool. The keel rasp head is parallel relative to the guide head and the first one or more protrusions of the keel rasp head is coaxially aligned with the second one or more protrusions of the guide head. The first one or more protrusions comprise a plurality of sharp teeth. The first one or more protrusions comprise a plurality of sharp teeth and the second one or more protrusions comprise a plurality of flat and smooth surfaces. The second one or more protrusions of the guide head of the movable guide assembly is sized and configured to be disposed into a keel channel. The guide head comprises a first contacting surface and a second contact surface, at least a portion of the first contact surface may contact or engage a bone surface.

In another embodiment, the keel alignment system comprising: a handle; a keel rasp disposed in a first plane, the keel rasp comprising a keel rasp head, the keel rasp head comprising a first one or more protrusions or keels, the first one or more protrusions or keels extending towards the superior direction, the keel rasp is removably coupled to the handle; and a guide tool assembly disposed in a second plane, the movable guide tool assembly comprising a guide arm, the guide arm comprising a guide head and a hand grip, the guide head comprising a second one or more protrusions or keels, the second one or more protrusions or keels extending towards the inferior direction, at least a portion of the guide tool assembly removably and pivotally coupled to the handle allowing the first one or more protrusions or keels of the keel rasp to be coaxially aligned to the second one or more protrusions or keels of the guide tool assembly; the guide tool assembly movable relative to the keel rasp from a first position, the first position comprising the keel rasp head is spaced apart at a first distance (D1) from the guide head, to a second position, the second position comprising the keel rasp head is spaced apart at a second distance (D2), the second distance being greater than the first distance. (See FIGS. 20F and 20G.)

Desirably, during use the assembled tool can be inserted between the vertebral bodies in a closed or collapsed condition (FIG. 21A), and then the handle depressed to separate the keel rasp head and the guide tool (FIG. 21B). the keel tool can them be advanced or otherwise operated (e.g., reciprocated) to create a desired keel channel (FIG. 21C), and then the tool removed after creation of aligned keel channels (FIG. 21D).

The handle comprises a powered handle or a manual handle. The keel rasp head is parallel relative to the guide head. The first one or more protrusions of the keel rasp head of the keel rasp comprise a plurality of sharp teeth. The second one or more protrusions comprise a plurality of flat and smooth surfaces. The second one or more protrusions of the guide head of the movable guide assembly is sized and configured to be disposed into a keel channel. The guide head comprises a first contacting surface and a second contact surface, at least a portion of the first contact surface may contact or engage a bone surface.

In another embodiment, the method of creating aligned keel channels comprises the steps of: coupling a portion of a guide tool assembly and a portion of a keel rasp to a handle to create an keel alignment system; aligning the keel of the guide arm of the guide tool assembly to follow or match a first keel channel on a first vertebral body; translating the keel of the guide arm of the guide tool assembly along the first keel channel of the first vertebral body until the translation stops; compressing or squeezing a second end or a portion of the guide tool assembly towards the handle to create a distance or a spacing at the first end between the guide head of the guide arm relative to the keel head of the keel rasp; stamping or punching a second keel channel on a second vertebral body.

In another embodiment, the method of creating aligned keel channels comprises the steps of: coupling a portion of a guide tool assembly and a portion of a keel rasp to a handle to create an keel alignment system; aligning the keel of the guide arm of the guide tool assembly to follow or match a first keel channel on a first vertebral body; compressing or squeezing a second end or a portion of the guide tool assembly towards the handle to create a distance or a spacing at the first end between the guide head of the guide arm relative to the keel head of the keel rasp; translating the keel of the guide arm of the guide tool assembly along the first keel channel of the first vertebral body until the translation stops to create a second keel channel on a second vertebral body.

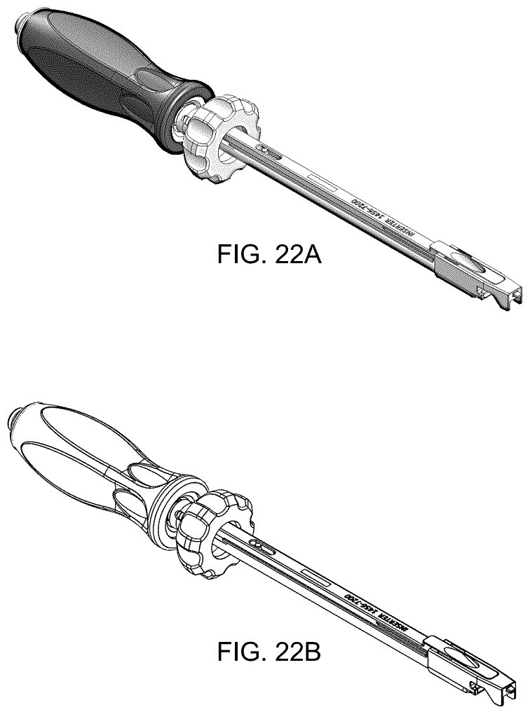

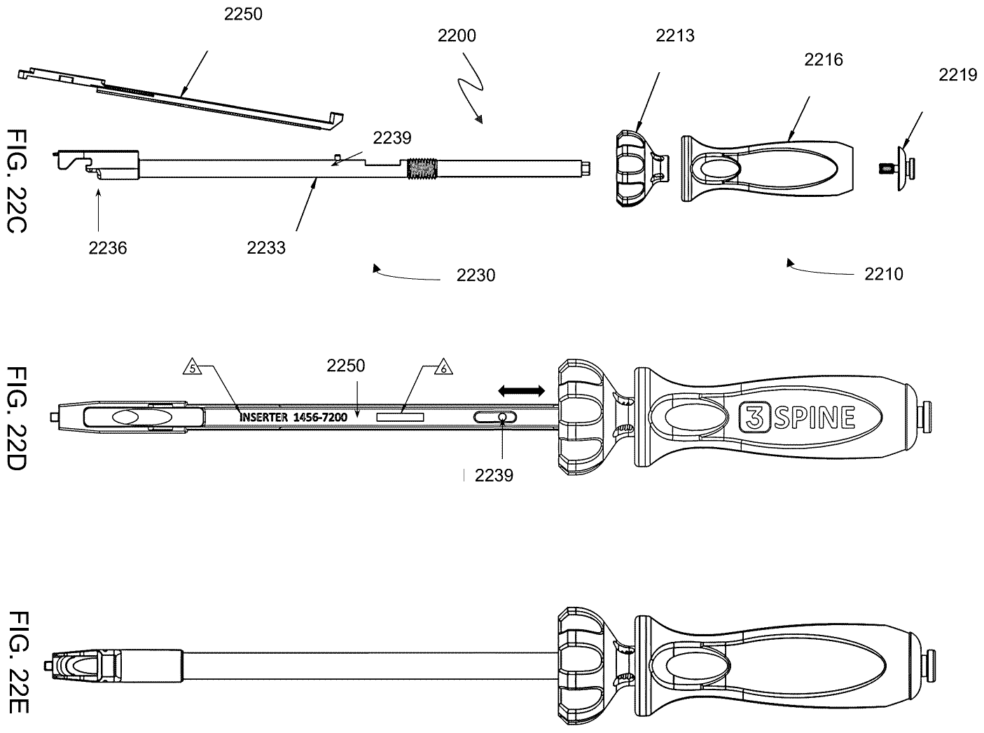

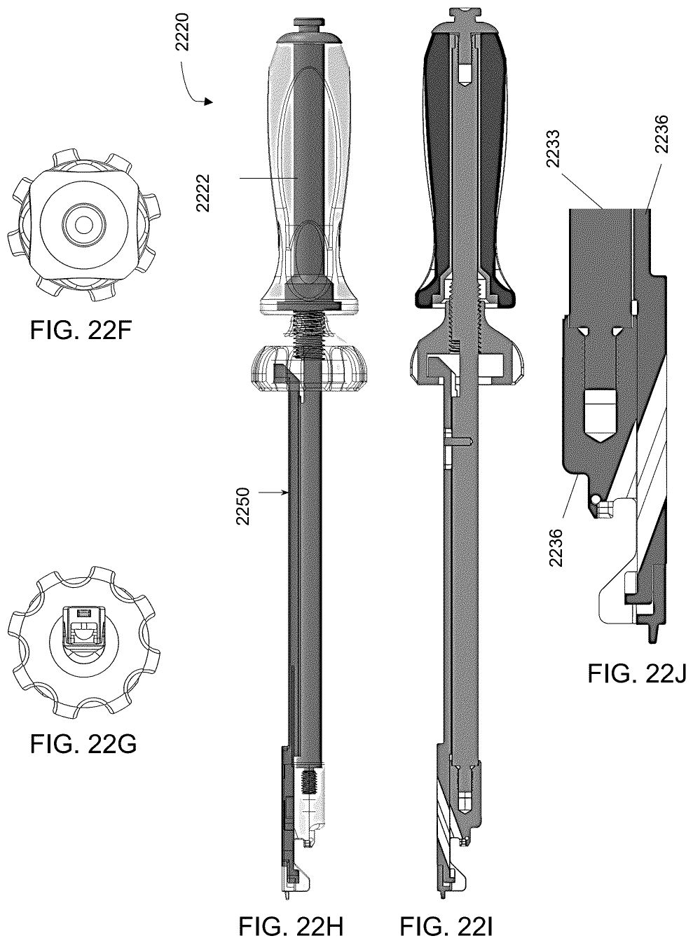

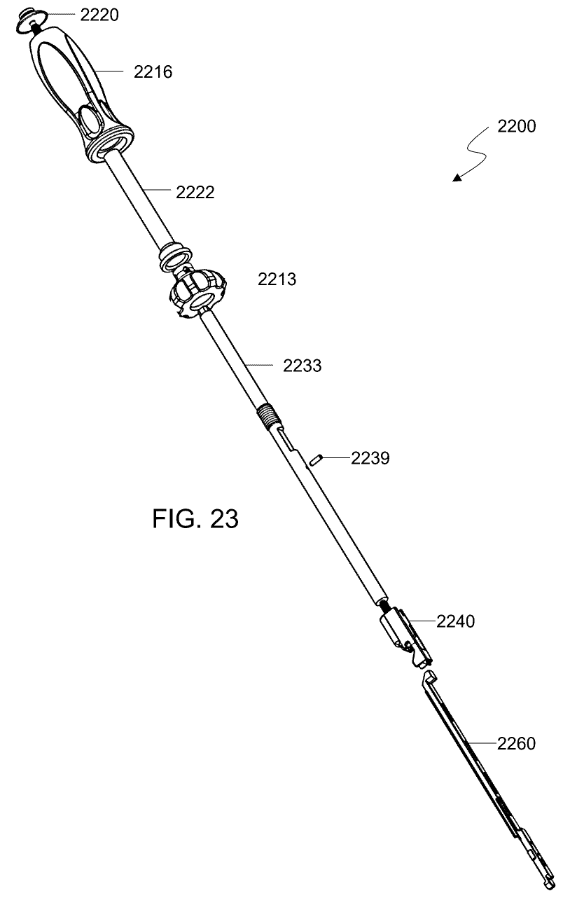

FIGS. 22A-22J depicts various plan views and cross-sectional views of one embodiment of an implant deployment or inserter assembly 2200, and FIG. 23 depicts an exploded isometric view of the implant deployment assembly 2200. The implant deployment assembly is an instrument used to help deploy and/or insert an implant or a spinal implant that aligns with the first prepared surface of a first vertebral body and a second prepared surface of the second vertebral body within an intervertebral space on at least one side. The first vertebral body and/or the second vertebral body may comprise the inferior or superior vertebral body. The implant deployment assembly may accommodate different implant sizes. The at least one side may further comprise a first side and a second side.

The implant deployment system may comprise an implant and an implant deployment assembly. The implant deployment system may further optionally include a screw guide. The implant deployment assembly 2200 comprises a handle subassembly 2210, an actuating subassembly 2230, and a draw bar 2260. The handle subassembly 2210 comprises a locking knob 2213, a handle or handgrip 2216, and an end cap 2220. The handle subassembly further comprises a handle core 2222. The actuator or actuating subassembly 2230 comprises an actuation rod 2233, a grasping tip 2240 and a dowel pin 2239.

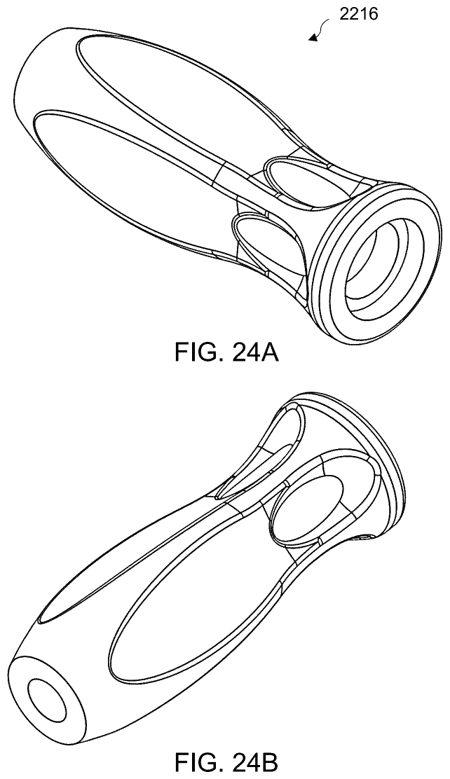

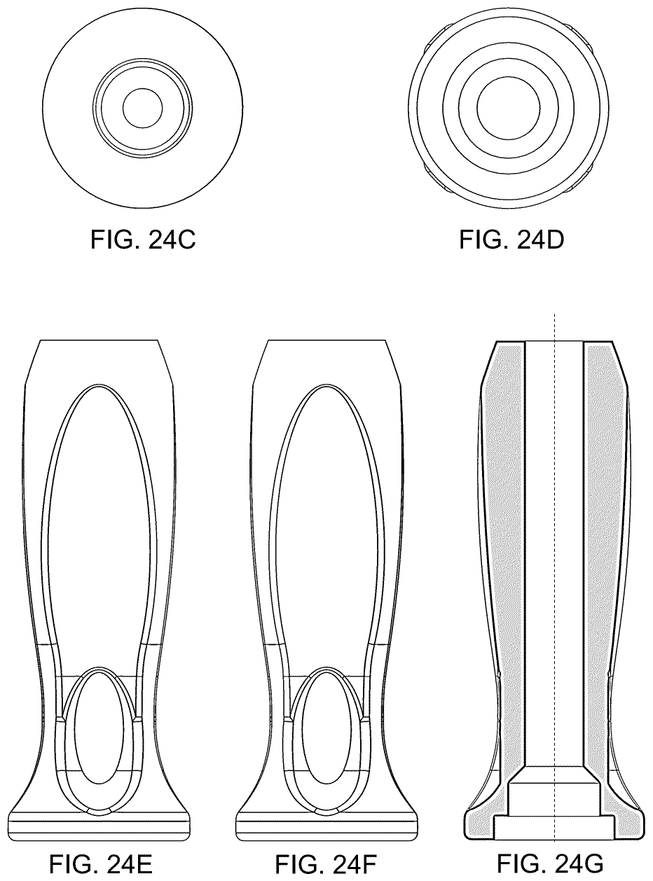

The handle or handgrip 2216 comprises a longitudinal axis, a length, an outer diameter or surface and an inner diameter or surface as shown in FIGS. 24A-24G. Alternatively, the handle or handgrip may comprise a longitudinal axis, an outer surface and a lumen or bore. The inner diameter, inner surface, bore or lumen extends along at least a portion of the length of the handle or handgrip. The inner diameter, inner surface, bore or lumen extends along or follows along the longitudinal axis of the handle or handgrip. The inner diameter, inner surface, bore or lumen extends from the bottom surface of the handle or handgrip through the top surface to create a first and second opening.

The inner diameter, inner surface, bore or lumen of the handle or handle grip comprises a first portion and a second portion. The second portion comprises a second portion length and diameter. The first portion comprises a first portion length and one or more first portion diameters. The one or more first portion diameters are larger than the second portion diameter. The second portion diameter is larger than the outer diameter the shaft of the handle core. The second portion diameter is sized and configured to receive a portion of the handle core. The second portion diameter is sized and configured to receive the shaft portion, second portion or second end of the handle core. The first portion is sized and configured to receive the plunger portion, the first portion or the first end of the handle core.

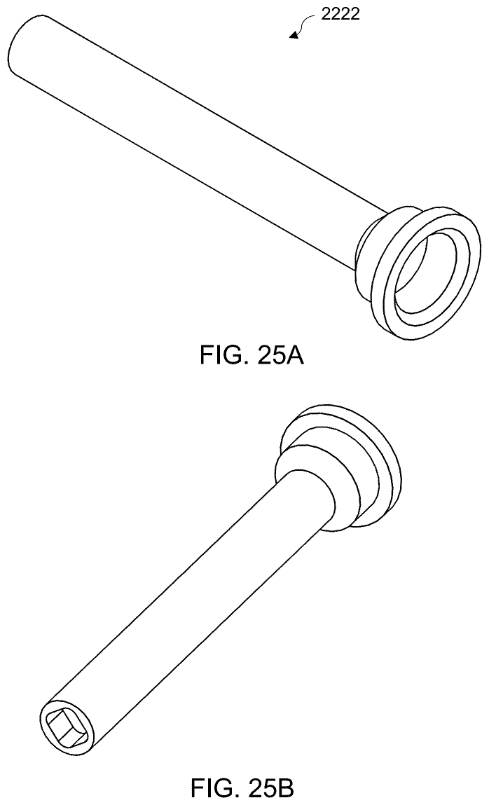

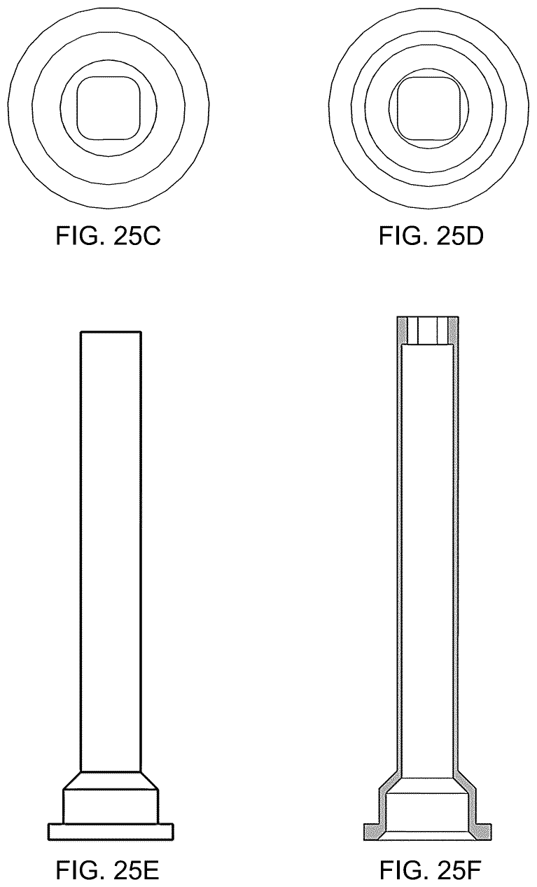

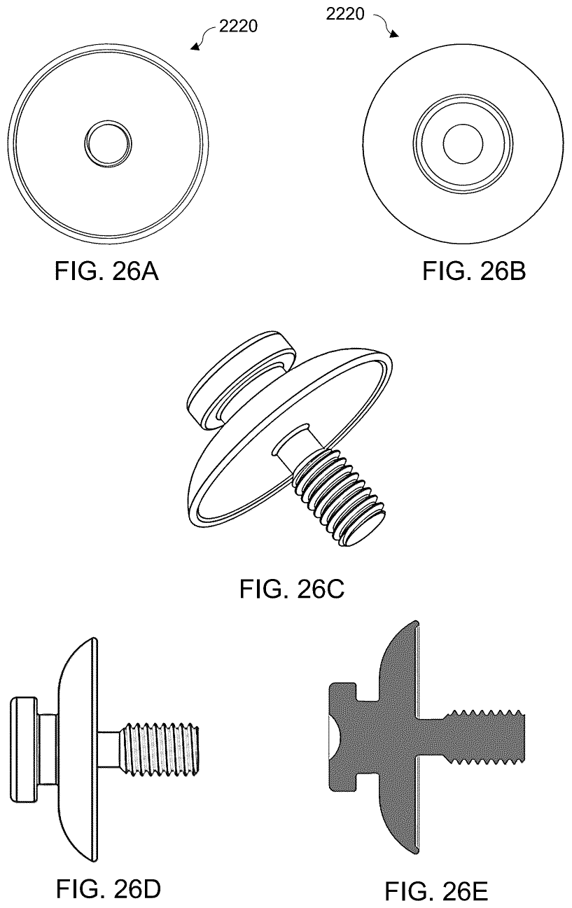

The handle subassembly further comprises a handle core 2222 as shown in FIGS. 25A-25F. The handle core comprises a longitudinal axis, a length, an outer diameter or surface and an inner diameter or surface. Alternatively, the handle core may comprise a longitudinal axis, an outer surface and a lumen or bore. The inner diameter, inner surface, bore or lumen extends along at least a portion of the length of the handle core. The inner diameter, inner surface, bore or lumen extends along or follows along the longitudinal axis of the handle core. The inner diameter, inner surface, bore or lumen extends from the bottom surface of the handle core through the top surface to create a first and second opening.

The inner diameter, inner surface, bore or lumen of the handle core comprises a first portion, a second portion and a third portion. The second portion comprises a second portion length and diameter. The third portion comprises a third portion length and diameter. The first portion comprises a first portion length and one or more first portion diameters. The one or more first portion diameters are larger than the second portion diameter. The second portion diameter is larger than the third portion diameter. The second portion is disposed between the first and third portion. The second portion diameter is larger than at least a portion of the outer diameter the actuating shaft of the actuating subassembly. The second portion diameter is sized and configured to receive a portion of the actuating shaft of the actuating subassembly and/or to receive a portion of the second end of the actuating shaft of the actuating subassembly. The first portion is sized and configured to receive a portion of the locking knob and/or a portion of the second end of the locking knob. The third portion is sized and configured to receive a portion of the end cap and/or to receive the threaded portion of the end cap. The third portion may further comprise threads.

FIGS. 26A-26E depicts various plan views of one embodiment of an end cap 2220. The end cap is used to secure the handle core to the handle or handgrip. The end cap comprises a head and a shank. The head comprises a bottom surface and a top surface. The top surface comprises a shape, the shape is convex, hemispherical or arched. The bottom surface is flat or planar. At least a portion of the bottom surface of the end cap contacts at least a portion of the handle or handgrip. The shank comprises at least a portion that is threaded. The shank comprises a first and second portion. The first portion is adjacent to the bottom surface of the head. The second portion comprises threads. The second portion is coupled to the handle core. The second portion is disposed within the third portion of the handle core and coupled by engaging the threads to secure the handle core to the handle or handgrip. The endcap may comprise a material. The material includes metal, polymer or rubber. The polymer can be a thermoset or thermoplastic. The metal may include stainless steel, a stainless steel alloy, titanium, titanium alloy, cobalt chrome, cobalt chrome alloy, nitinol, and/or tantalum.

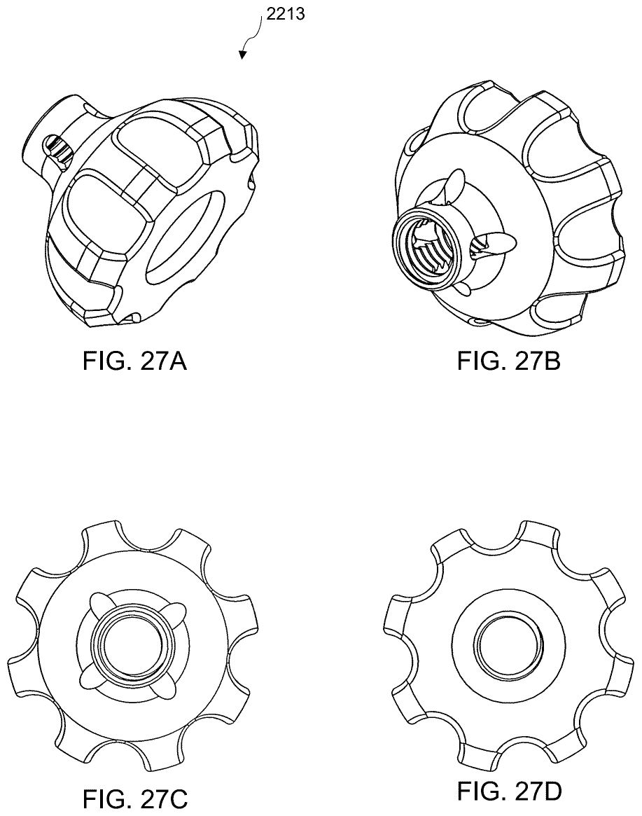

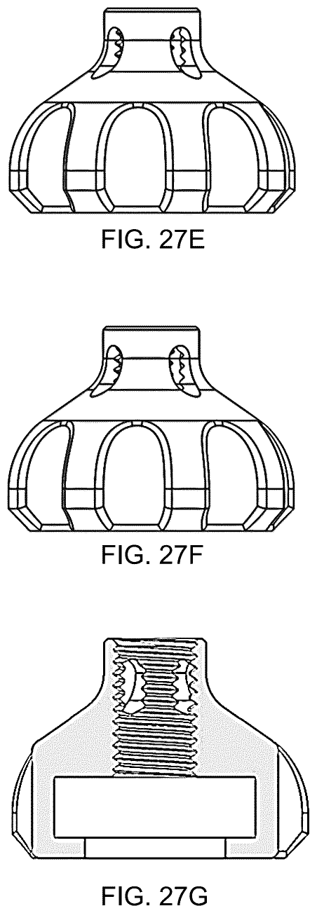

FIGS. 27A-27G depicts various plan views of one embodiment of a locking knob 2213 of the handle subassembly. The locking knob of the implant deployment assembly or implant inserter assembly is a mechanism that rotates relative to a handle grip or handle of the implant deployment assembly or implant inserter assembly. The locking knob is linked to actuation subassembly and the draw bar. The rotation of the locking knob turns the rotary motion into linear motion by translating the actuation assembly relative to the longitudinal axis of draw bar. The draw bar is fixed to the locking knob allowing the draw bar to translate linearly along the longitudinal axis of the implant deployment assembly to disengage and engage the implant and/or lock or unlock the implant to the actuation subassembly or implant deployment sub assembly. More specifically, the rotation of the locking knob in a clockwise direction translates the actuation subassembly relative to the draw bar to translate the draw bar in a proximal direction inside the intervertebral space (towards anterior direction) to allow the draw bar to contact a portion of the implant to deploy outwardly relative to the longitudinal axis implant deployment subassembly; and the rotation of the locking knob in a clockwise direction translates the actuation subassembly relative to the draw bar to in a distal direction (towards posterior direction) until a portion of the implant or the posterior end of the implant contacts a portion of the actuation subassembly or the grasping tip.

The locking knob comprises a body, and a stem. The body comprises an outer surface and a shape. The outer surface comprises one or more knurls, flutes or lobes. The shape comprises generally hemispherical or rounded shape. The body further comprises a first end and a second end. The stem is disposed at a second end. The stem comprises a stem diameter that is smaller than the body diameter. The stem is sized and configured to be disposed into a first portion of the handle core. The locking knob further comprises a material. The material includes metal, polymer or rubber. The polymer can be a thermoset or thermoplastic. The metal may include stainless steel, a stainless steel alloy, titanium, titanium alloy, cobalt chrome, cobalt chrome alloy, nitinol, and/or tantalum. In one embodiment, the material comprises a stainless steel alloy, the stainless steel alloy includes Nictronic 60.

The locking knob body further comprises a longitudinal axis, a channel, an inner diameter, inner surface, bore or a lumen. The inner diameter, inner surface, bore or lumen comprises threads. The inner diameter, inner surface, bore or lumen is sized and configured to receive a portion of the actuation subassembly. The threads of the inner diameter, inner surface, bore or lumen engage with the outer threads of the actuation rod of the actuation subassembly. The inner diameter, inner surface, bore or lumen comprises a diameter and a length. The length comprises the linear distance translated from rotary motion. The length includes 10 mm to 30 mm; the length includes 15 mm to 25 mm; and/or the length includes 18 mm to 22 mm.

The channel, recess or groove is proximate to the threaded inner diameter, inner surface, bore or lumen. The channel, recess or groove is sized and configured to receive a portion of the draw bar. The channel, recess or groove is sized and configured to receive a portion of second end of a draw bar. The channel, recess or groove further comprises a diameter. The diameter of the channel, recess or groove is larger than the diameter of the inner diameter, inner surface, bore or lumen. The channel, recess or groove further comprises a shoulder and/or one or more contact surfaces. At least a portion one or more contact surfaces of the channel, recess or groove contacts or engages one or more contact surfaces of the at least a portion of the second end of the draw bar. The channel, recess or groove is disposed radially within the body.

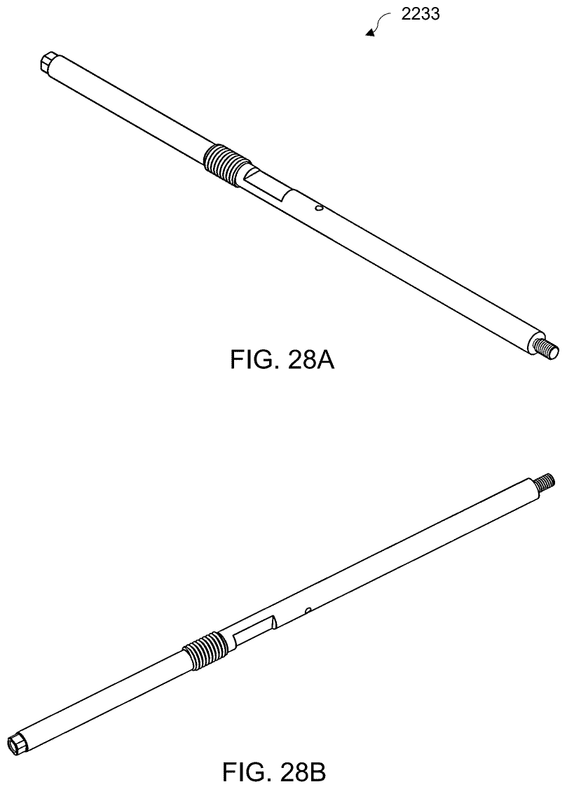

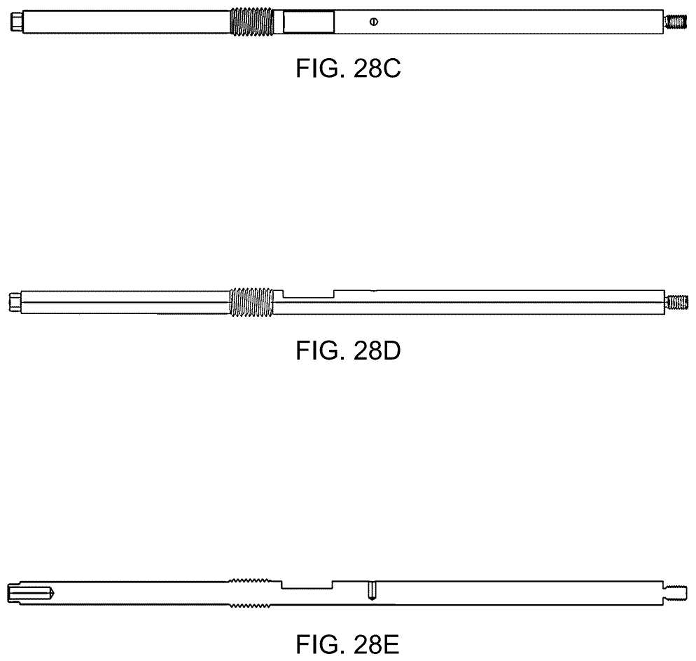

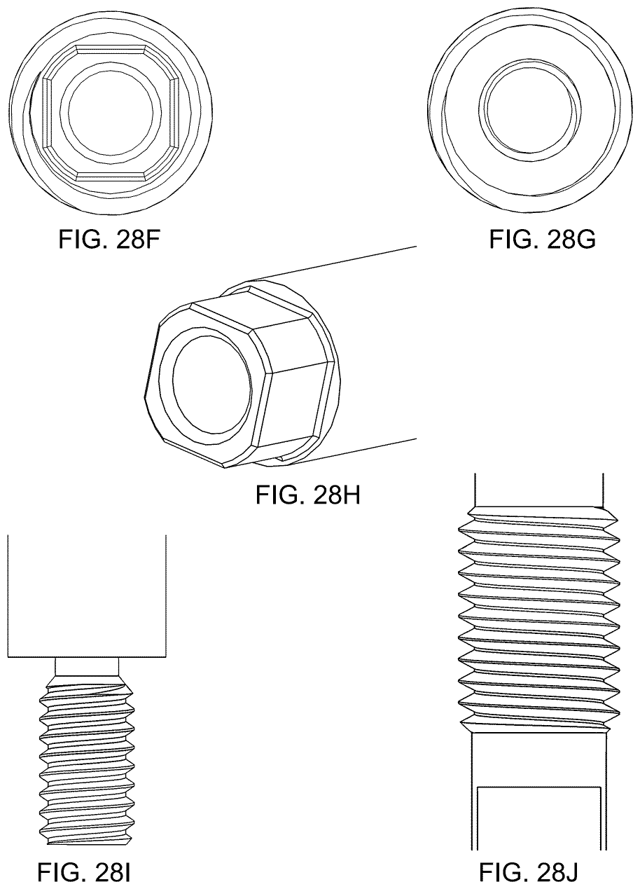

FIGS. 28A-28J depicts various plan views and cross-sectional views of one embodiment an actuation rod 2233 of the actuation subassembly. The actuation rod comprises a first end, a second end and a longitudinal axis. The actuation rod further comprises a first threads and a second threads. The first threads are disposed at a first end of the actuation rod. The first threads extend outward and aligned with the longitudinal axis of the actuation rod. The first threads are sized and configured to engage with the grasping tip. The first threads are disposed into the threaded bore of the grasping tip. The second threads are disposed on an outer surface of the actuation rod. The second threads are positioned between the first end and the second end. The second threads are sized and configured to be disposed within the threaded inner diameter, inner surface, bore or lumen of the locking knob. The second threads of the actuation rod contact and engage with the threads of the inner diameter, inner surface, bore and/or lumen of the locking knob to transform the rotatory motion into linear motion and allowing the actuation rod to move relative to the draw bar.

The actuation rod further comprises head, a recess and one or more openings. The head comprises a shape, the shape is hexalobular. The head is disposed at a second end of the actuation rod. The head is sized and configured to be disposed into a third portion of the handle core of the handle subassembly. The recess is disposed between the second threads and the first threads. The recess is positioned proximate or adjacent to the second threads. The recess is sized and configured to receive a portion of the draw bar. The recess is sized and configured to receive a portion of the second end of the draw bar.

The one or more openings may comprise a first opening and/or a second opening. The first opening is sized and configured to receive a portion of a dowel pin. The first opening is disposed between the recess and/or second threads and the first threads. The first opening is positioned proximate and/or adjacent to the recess. The first opening is comprises a longitudinal axis, the longitudinal axis of the first opening is transverse, orthogonal or 90 degrees to the longitudinal axis of the actuation rod. At least a portion of the second opening is disposed at the second end. At least a portion of the second opening is disposed within the head of the actuation rod. The second opening is coaxially aligned with the longitudinal axis of the actuation rod.

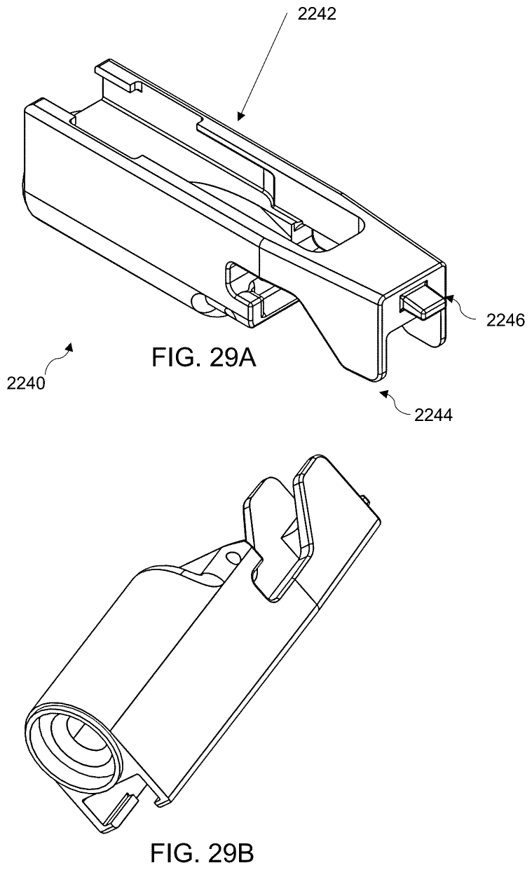

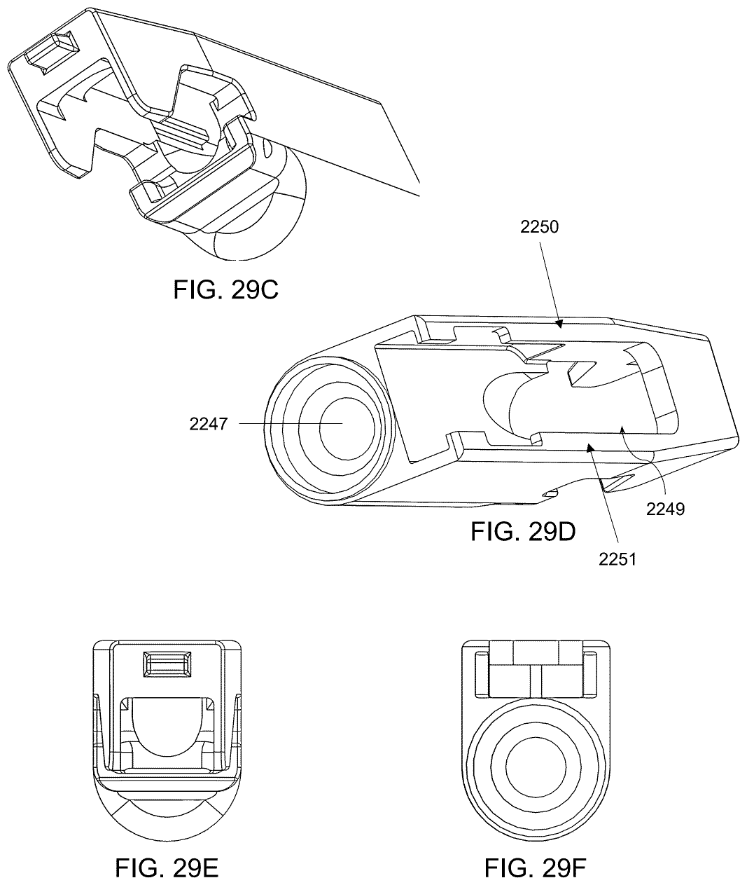

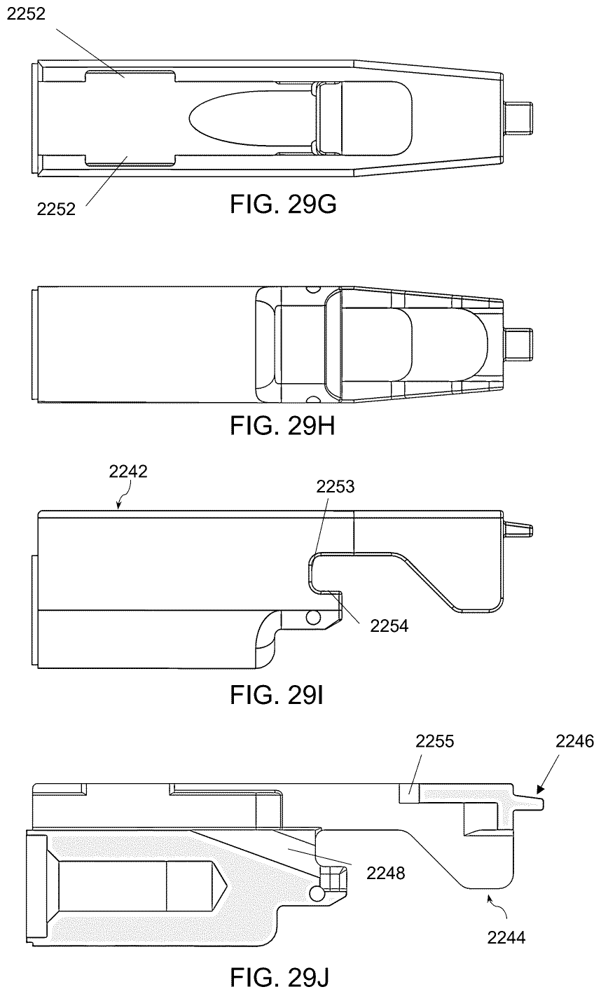

FIGS. 29A-29J comprises various plan views and a cross-sectional view of one embodiment of a grasping tip 2240 of the actuation subassembly. The grasping tip 2240 is used to engage or contact at least a portion of the implant to facilitate locking and unlocking and/or disengage or engage at least a portion of the implant. The grasping tip comprises a body 2242, a hook 2244 and a tab or protrusion 2246. The grasping tip further comprises a longitudinal axis, a first end and a second end. The body of the grasping tip comprises a first bore 2247, a second bore 2248 and a recess 2249. The first bore extends from a back surface towards the front surface. The first bore extends from a second end towards the first end. The first bore is parallel to the to the longitudinal axis of the grasping tip. The first bore is threaded.

The recess comprises a length, width, height, a first flange 2250 and a second flange 2251. The recess is disposed on at least a portion of the top surface of the body of the grasping tip or body. The recess extends from a top surface of the body or the grasping tip towards the bottom surface of the body or grasping tip. The recess height and width are sized and configured to receive a portion of the draw bar. The recess height and width are sized and configured to receive a portion of the 1a end of the draw bar. The recess further comprises a first flange 2250 and a second flange 2251. The first and second flange extends along the length of the recess and extends outwardly over at least a portion of the recessed opening. The first and second flange extend parallel to the top surface and/or co-planar to the top surface of the body or grasping tip. At least a portion of the first and second flanges contact or engage at least a portion of the draw bar or at least a portion of the top surface of the draw bar to prevent migration. The first and second flange comprises a first and second cutout 2252 respectively. The first and second cutouts are sized and configured to receive a portion of the one or more tabs, a first tab and/or a second tab of the draw bar.

The recess further comprises first contact surface and a second contact surface. At least a portion of the first end of the draw bar is inserted within the recess to allow a bottom surface of the draw bar to contact or engage a portion of the recess bottom surface. The draw bar will be translated within the recess of the grasping tip towards the first end of the grasping tip until the first contact surface of the first end of the draw bar contacts or engages the contact surface within the recess of the grasping tip. The contact or engagement of the contact surface of the draw bar to the contact surface of the recess of the grasping tip provides for the pushing and/or at least a portion of the implant and/or a second end of the implant to be deployed and/or provide a stop for distance translation.

The second bore extends from a bottom surface of the recess towards the bottom surface of the body or grasping tip. The second bore comprises a second longitudinal axis. The second longitudinal axis of the second bore is at a different orientation than the longitudinal axis of the grasping tip. The second longitudinal axis of the second bore is at oblique orientation compared to the longitudinal axis of the grasping tip. The second longitudinal axis of the second bore is at an angled orientation compared the longitudinal axis of the grasping tip. The angled orientation and/or the oblique orientation comprises matching or substantially matching the sagittal pedicle angle. The second bore is used as a guide opening to deploy a fastener or fixation screw through the second bore to couple the implant to the targeted anatomy.

The hook 2244 is disposed on the first end. The hook extends outwardly to be aligned with the longitudinal axis of the grasping tip. The hook further comprises a length or shoulder and/or a stop wall 2253 or stop contact wall. The ledge 2254 comprises a ledge surface that contacts or engages at least a portion of the implant and/or at least a portion of the second end of an implant. The ledge comprises a ledge surface that contacts or engages at least a portion of a bottom surface of the implant and/or at least a portion of the bottom surface at the second end of an implant. The stop wall or stop contact wall comprises a surface that contacts or engages a portion of an implant and/or at least a portion of the implant. The stop wall or stop contact wall comprises a surface 2255 that contacts or engages at least a portion of a back surface (or posterior surface) of the implant and/or at least a portion of the back surface (or posterior surface) at the second end of an implant. The hook may comprise a tapered shape.

The grasping tip further comprises a tab 2246. The tab is disposed at a first end of the grasping tip. Alternatively, the tab is disposed on a front surface of the hook or the front surface of the grasping tip. The tab extends substantially parallel or colinear with the longitudinal axis of the grasping tip. The tab extends parallel or colinear with the longitudinal axis of the grasping tip. The tab further comprises a top surface and a bottom surface. The top surface comprises an angled orientation. The top surface is angled relative to the longitudinal axis of the grasping tip. The bottom surface is flat and parallel to the longitudinal axis of the grasping tip. The angle of the top surface comprises a range of 5 to 10 degrees; and/or a range of 6 to 8 degrees. The tab of the grasping tip is sized and configured to be disposed within a recess of the implant and/or within a recess positioned at the second end of the implant. The tab of the grasping tip holds, engages a top portion of the implant or a superior component of the implant to facilitate longitudinal alignment, match or substantially match the longitudinal alignment of the implant deployment assembly. Furthermore, the tab of the grasping tip may help prevent migration or rotation of the top portion of the implant or the superior component of an implant.

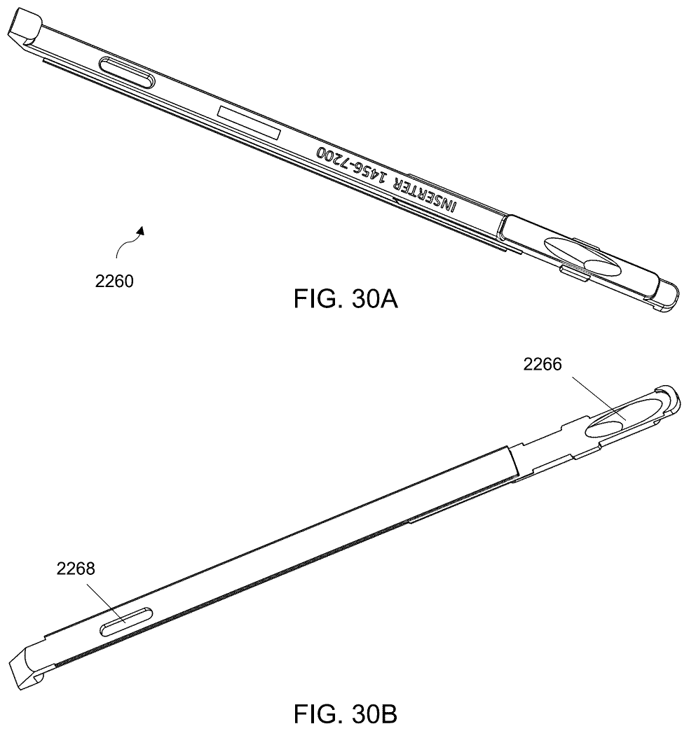

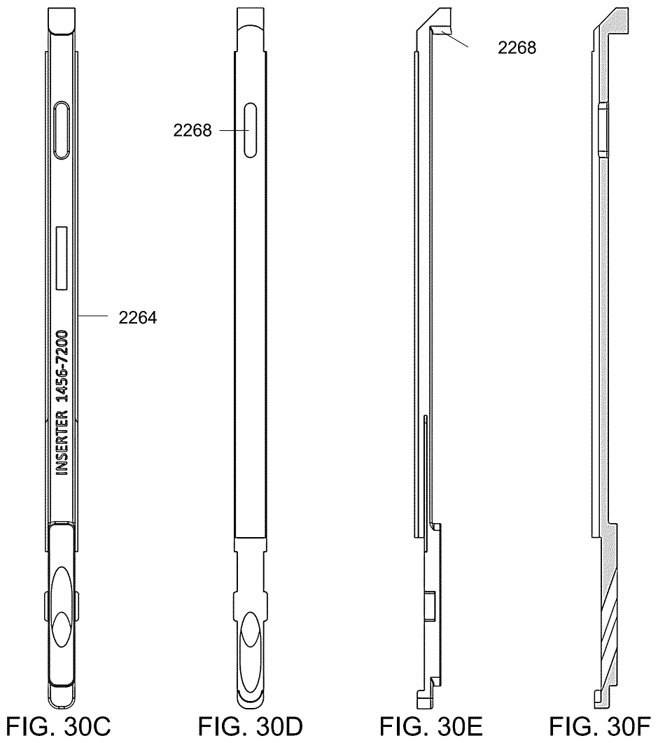

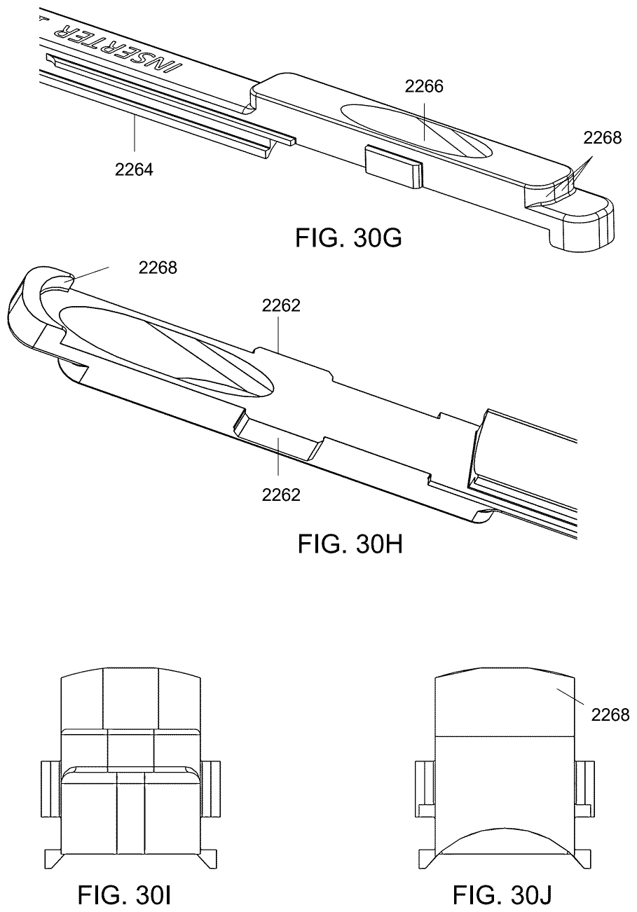

FIGS. 30A-30J depict various plan views and a cross-sectional view of one embodiment of a draw bar 2260. The draw bar is a component coupled to the actuation subassembly to allow the deployment of the implant into the targeted anatomical area or region. The draw bar desirably remains stationary relative to the actuation assembly as it moves relative to the draw bar. This translation allows the draw bar to push or pull and/or engage or disengage the implant. The draw bar comprises a longitudinal axis, a first end, a second end, a top surface and a bottom surface. The draw bar further comprises one or more tabs 2262, one or more rails 2264, a bore 2266 and a slot 2268. The draw bar further comprises a plurality of contact surfaces and/or a first contact surface, a second contact surface, a third contact surface and/or a fourth contact surface.

The draw bar comprises a slot 2268 disposed between a first end and a second end of the draw bar. The slot is positioned proximate or adjacent to the second end of the draw bar. The slot is sized and configured to receive at least a portion of a dowel pin. The slot is used to control the translation distance for deployment of the implant into the targeted anatomical area or region. The distance comprises a range of 5 to 20 mm; the distance comprises a range of 5 to 15 mm; and/or the distance comprises a range of 13-18 mm. The slot extends through from a top surface of the draw bar to the bottom surface. The slot is aligned with the longitudinal axis of the draw bar.

The draw bar comprises a bore 2266 which extends from atop surface of the draw bar towards the bottom surface of the draw bar. The bore of the draw bar comprises a second longitudinal axis. The second longitudinal axis of the bore of the draw bar is at a different orientation than the longitudinal axis of the draw bar. The second longitudinal axis of the bore of the draw bar is at oblique orientation compared to the longitudinal axis of the draw bar. The second longitudinal axis of the bore of the draw bar is at an angled orientation compared the longitudinal axis of the draw bar. The angled orientation and/or the oblique orientation comprises matching or substantially matching the sagittal pedicle angle. The second longitudinal axis of the bore of the draw bar is coaxially aligned and/or substantially coaxially aligned with the second longitudinal axis of the bore of the grasping tip. The second longitudinal axis of the bore of the draw bar is concentric aligned and/or substantially concentric with the second longitudinal axis of the bore of the grasping tip. The bore of the draw bar is positioned between the first end and the second end. The bore of the draw bar is positioned proximate or adjacent to the first end. The bore of the draw bar is used as a guide opening to deploy a fastener or fixation screw through the second bore to couple the implant to the targeted anatomy.

The draw bar further comprises one or more tabs 2262 and/or a first and second tab. The one or more tabs are positioned at the first end of the draw bar. The one or more tabs, a first tab or second tab are positioned adjacent or proximate to the bore of the draw bar. The one or more tabs are positioned between the first end and the second end. The one or more tabs, a first tab and/or a second tabs extend outwardly from a first and second side surfaces. The one or more tabs, a first tab and/or a second tab are sized and configured to be disposed within the first and second cut-outs of the first and second flange of the recess of the grasping tip.

The draw bar further comprises one or more plurality of contact surfaces 2268. The one or more plurality of contact surfaces may comprise a first contact surface, a second contact surface, a third contact surface and a fourth contact surface. At least a portion of the first contact surface of the draw bar engages or contacts with at least a portion of the contact surface of the grasping tip. At least a portion of the second contact surface of the draw bar engages or contacts with at least a portion of the second end of the implant. At least a portion of the third contact surface contacts or engages with at least a portion of the shoulder and/or at least a portion of the ledge surface or second surface of the shoulder or ledge of the recess, groove or channel of the locking knob. At least a portion of the fourth contact surface of the draw bar contacts or engages at least a portion of the recess, groove or channel of the locking knob. At least a portion of the fourth contact surface of the draw bar contacts or engages at least a portion of the 1a contact surface of the recess, groove or channel of the locking knob.

The draw bar further comprises a plurality of rails and/or a first rail and a second rail. The plurality of rails, the first rail and/or the second rail is disposed between the first end and the second end. The plurality of rails, the first rail and/or the second rail extend outwardly from the first and second sides of the draw bar. The plurality of rails, the first rail and/or the second rail extend along a portion of the length of the draw bar. The plurality of rails, the first rail and/or the second rail are sized and configured to be disposed into one or more channels of the screw guide. The plurality of rails, the first rail and/or the second rail may comprise a single rail. Alternatively, each of the plurality of rails, the first rail and/or the second rail may comprise a top rail and a bottom rail.

The draw bar further comprises a top surface and a bottom surface. At least a portion of the top surface comprises a flat or planar surface. At least a portion of the top surface comprises a curved surface. At least a portion of the bottom surface comprises a flat or planar surface. At least a portion of the bottom surface comprises a curved surface. The curved surface comprises a concave, arched and/or hemispherical shape. The curved surface may be sized and configured to receive a portion of the actuation rod of the actuation subassembly.

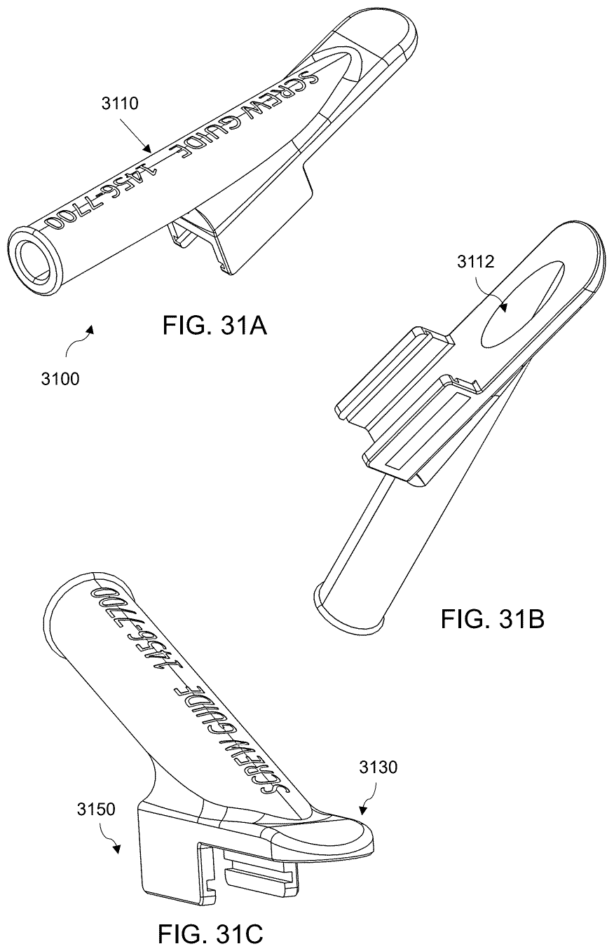

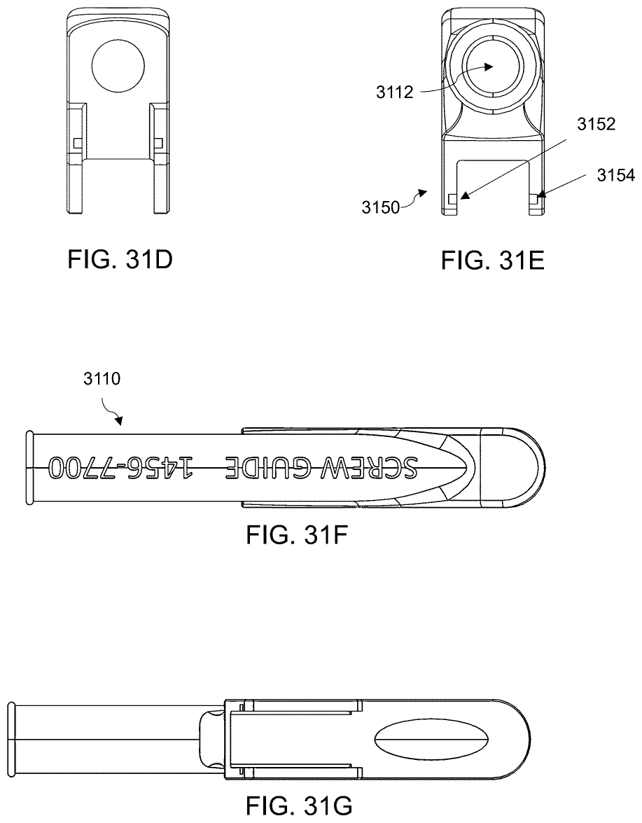

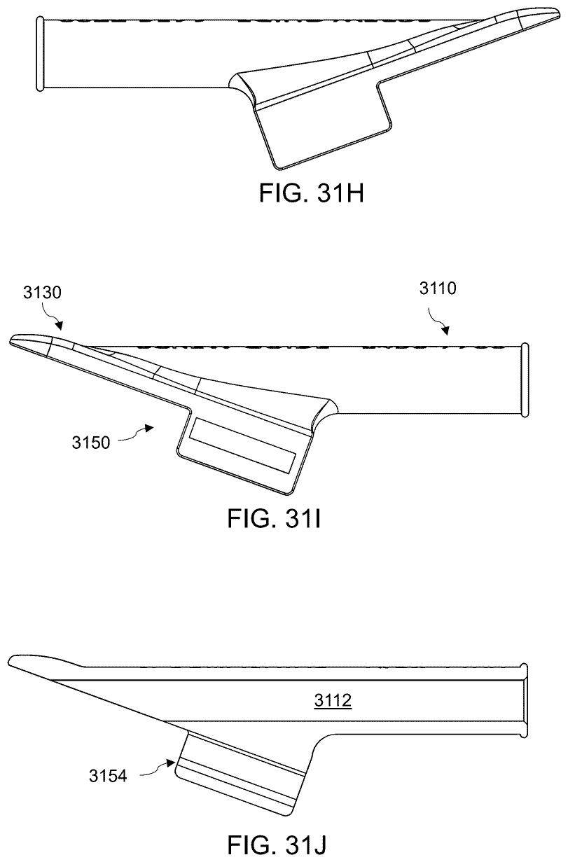

FIGS. 31A-31J depicts various plan views and across-sectional view of one embodiment of a screw guide 3100. The screw guide is a tool or component used to center a surgical fixation screw that will be used to secure the implant to the targeted anatomy. The bore of the screw guide is designed to accommodate the external diameter of the fixation screw. The screw guide comprises a guide tube or barrel 3110, a base 3130 and a track 3150. The base comprises a top surface, a bottom surface, a first end and a second end. At least a portion of the top and/or bottom surface of the base is flat or planar. At least a portion of top surface is angled. At least a portion adjacent to the first end of the base is angled.

The screw guide comprises a guide tube or barrel. The guide tube or barrel comprises an outer diameter and a bore 3112. The guide tube or barrel extends away from the base. The guide tube or barrel extends from a top surface of the base. The guide tube or barrel extends away from the base at an angled orientation. The guide tube or barrel extends away from a top surface of the base at an angled orientation. The angled orientation may match or substantially match a sagittal pedicle angle. The angled orientation may comprise 10 degrees to 30 degrees angle; the angled orientation may comprise 15 degrees to 25 degrees angle; and/or the angled orientation may comprise a 18 degrees to 22 degrees angle. The bore 3112 may extend along the length of the bore and through the bottom surface of the base. The bore may be sized and configured to receive a portion of a fixation screw.

The screw guide may comprise a track 3150. The track comprises a track width, a first wall and a second wall. Each of the first and second walls of the track extends from a portion of the base downwardly. Each of the first and second walls of the track extends from a portion of the bottom surface of the base downwardly. Each of the first and second walls of the track extends orthogonally downward from a portion of the bottom surface of the base. The first wall comprises a first channel 3152 and the second wall comprises a second channel 3154. The first channel and/or the second channel are sized and configured to receive a portion of plurality of rails, the first rail and/or the second rail of the draw bar. Alternatively, the first channel and/or the second channel are sized and configured to receive a portion of the top rail and/or bottom rail of the plurality of rails, the first rail and/or the second rail. The track width is sized and configured to receive a portion of a width of the draw bar.

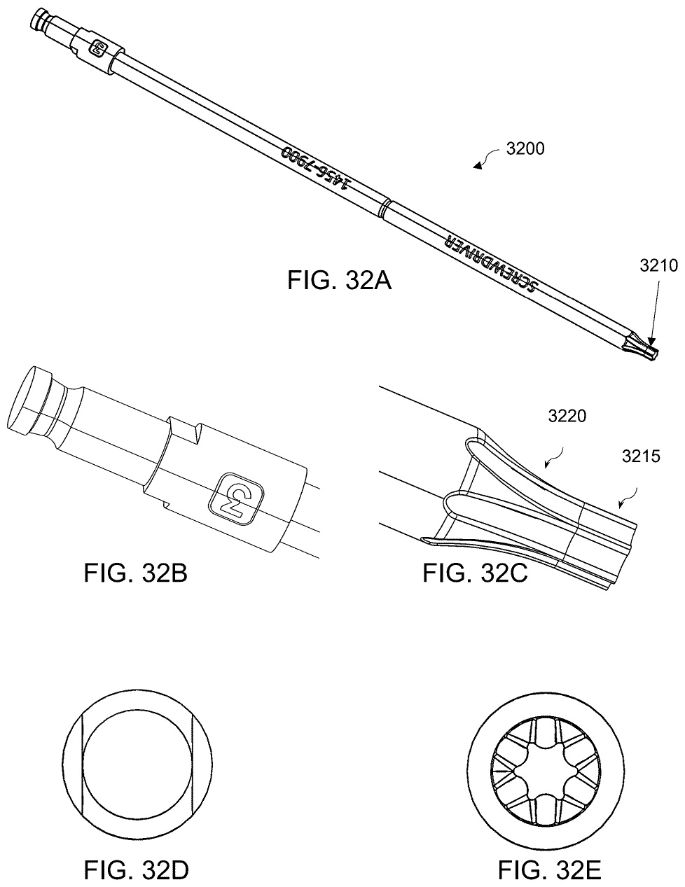

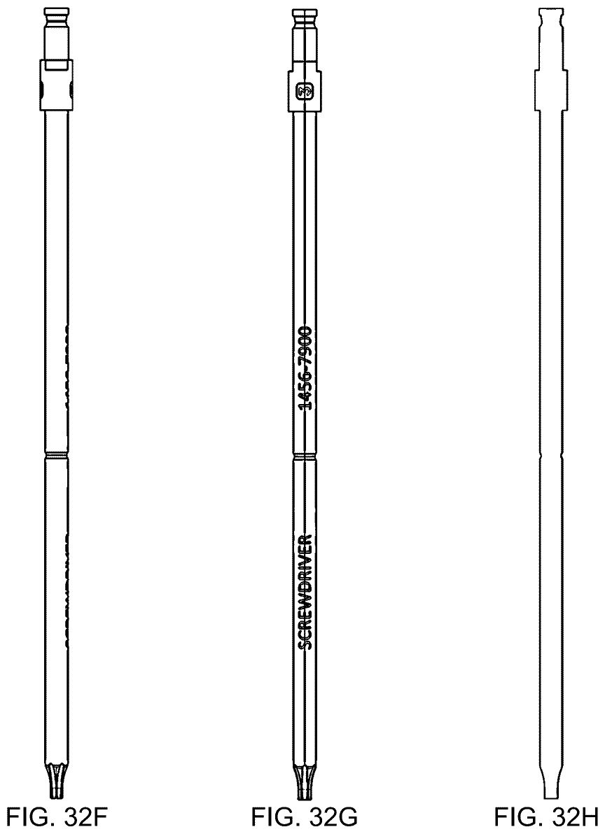

FIGS. 32A-32H depicts different plan views and across-sectional view of embodiment of a screwdriver 3200. The screwdriver tool allows the surgeon or user to insert or remove fixation screws or fasteners. The screwdriver tool tip engages with the driving head of a fixation screw or fastener to allow the surgeon or user to provide a turning force or torque in a clockwise or counterclockwise direction for insertion or removal. The screwdriver comprises a driving tip, a shaft and a connector. The screwdriver further comprises a longitudinal axis, a first end and a second end.

The driving tip 3210 of the screwdriver comprises a first portion 3215, a second portion 3220, and/or a total length. The driving tip is disposed at the first end of the screwdriver. The total length of the driving tip comprises 5 mm to 10 mm length; the total length of the driving tip comprises 8 mm to 9 mm; and/or the total length of the driving tip comprises greater than 8 mm. The driving tip first portion comprises a first portion length and first portion diameter. The driving tip second portion comprises a second portion diameter and a second portion length. The first portion diameter is smaller than the second portion diameter. The second portion of the driving tip is tapered. The first portion of the driving tip is straight. The first portion of the driving tip is disposed and engaged within the driving recess of the head of the fixation screw or fastener. The first portion length comprises a shorter length than the second portion length. The driving tip is tapered to allow a transition between the driving tip and the shaft. The driving tip comprises a driving type, the driving type includes a flathead, a Phillips, a Pozidrive, a Hex or Hex Key or Allen, a Torx, square head, a Tri-point and/or any combination thereof. In one embodiment, the driving type comprises a hex or hex key or hexalobular type.

The screwdriver may comprise a shaft. The shaft comprises a first end, a second end, a length, and/or a diameter. The diameter of the shaft may comprise a range of 5 mm to 8 mm; the diameter may further comprise a range of 6 mm to 8 mm; the diameter may further comprise a range of 5 mm to 6 mm. The diameter of the shaft may be uniform along the longitudinal axis and/or length of the shaft. The shaft may comprise a shape, the shape may be cylindrical or generally cylindrical. The length of the shaft may comprise a range of 125 mm to 175 mm; the length of the shaft may comprise a range of 140 mm to 165 mm; the length of the shaft may comprise a range of 158 mm to 162 mm; and the length of the shaft may be greater than 155 mm. The driving tip is disposed at a first end, and the connector disposed at a second end. The shaft may be solid or hollow.

The screwdriver may comprise a connector, which may include a male and/or a female connector. The connectors may be configured to engage with a Hudson connection, an Association for Osteosynthesis (AO) connection and/or a Trinkle connection. The connection mechanisms comprise quick-connect and quick-disconnect. The connector may be disposed within and/or coupled to a manual handle and/or a powered handle or handpiece.

The screwdriver and/or screwdriver shaft may further comprise one or more reference markers. The reference markers are used when the screwdriver is inserted into the intervertebral disc space to help guide the surgeon and/or user on positioning of the screwdriver tool relative to the intervertebral disc space. The one or more reference markers may be confirmed radiographically to determine the length of insertion of the screwdriver into the intervertebral disc space to achieve the fixation to the targeted anatomical region. The one or more reference markers may comprise a radiopaque material or be radiopaque to allow visualization with an imaging equipment. The one or more reference markers may comprise a laser marking, a ring, a channel, and/or an opening. The one or more reference markers may be disposed onto a portion of the head and/or the shaft.

The actuation rod, the grasping tip, the draw bar, the screw guide and the screwdriver further comprise a material. The material includes a metal, a polymer, and/or ceramic. The material may include a polymer, a metal and/or a ceramic. The polymer may include a thermoset or thermoplastic. The metal may include stainless steel, a stainless-steel alloy, titanium, titanium alloy, cobalt chrome, cobalt chrome alloy, nitinol, and/or tantalum. The actuation rod, grasping tip, the draw bar, the screw guide and/or screwdriver further comprises a surface texture. The surface texture comprises a rough or polished texture. The roughened surfaces or porous surfaces, including turned, blasting, sand blasting, acid etching, chemical etching, dual acid etched, plasma sprayed, anodized surfaces, and/or any combination thereof. The polished surface may be accomplished different techniques, mechanical polishing, chemical polishing, electrolytic polishing, and/or any combination thereof. The polished surfaces can be measured in “Ra” micrometers (μm) or microinches (μin.). The Ra may comprise a range of 0.025 to 1.60 μm; may comprise a range of 0.025 to 0.40 μm; and/or may comprise a range of 0.20 o 0.40 μm. Accordingly, the Ra may comprise at least 0.05 μm or higher; at least 0.20 μm or higher and/or at least 0.4 μm or higher.

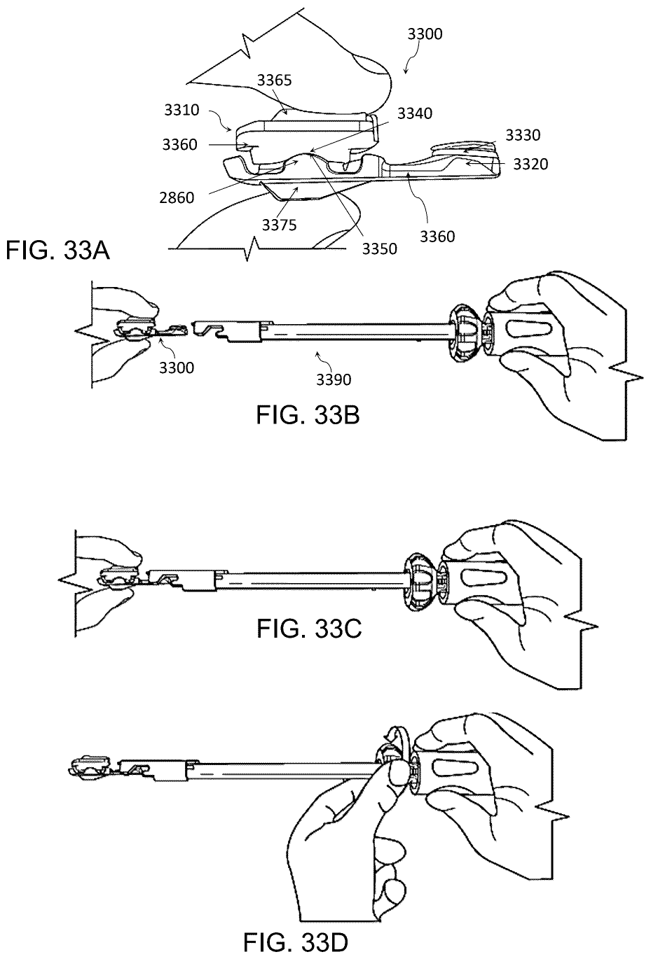

FIGS. 33A-33D, 34A-34E and 35A-35E depict various side views and cross-sectional views of one embodiment of a method of loading and/or using the implant deployment system. As previously described herein, the implant deployment system may comprise an implant and an implant deployment assembly. The implant deployment system may further comprise a screw guide and/or a screwdriver. The implant deployment assembly comprises a handle subassembly, an actuating subassembly, and a draw bar. The handle subassembly comprises a locking knob, a handle or handgrip, and an end cap. The handle subassembly further comprises a handle core. The actuator or actuating subassembly comprises an actuation rod and a grasping tip. The actuator or actuating subassembly further comprises a dowel pin. The implant may comprise a spinal implant.

In one embodiment, the method of loading the implant deployment system comprises the steps of: selecting the proper or desired sized implant; assembling the implant deployment assembly, the implant deployment assembly comprising a draw bar, an actuation subassembly and a handle subassembly; engaging the grasping tip of the deployment assembly onto a portion of a second end or posterior end of the implant; and rotating a locking knob of the implant deployment assembly to allow an actuation subassembly to move or translate posteriorly relative to the draw bar to lock the implant and prevent movement. The implant comprising a spinal implant. The spinal implant comprising a superior component and an inferior component.

In another embodiment, the method of loading the implant deployment system comprises the steps of: selecting the proper or desired sized implant; assembling the implant deployment assembly, the implant deployment assembly comprising a draw bar, an actuation subassembly and a handle subassembly; engaging a hook of the grasping tip of the deployment assembly onto a portion of a second end or posterior end of the implant; and rotating a locking knob of the implant deployment assembly to allow an actuation subassembly to move or translate posteriorly relative to the draw bar until the second or posterior end of the implant contacts or engages a stop wall of the grasping tip and/or the protrusion of the grasping tip is disposed into a recess of the implant. The implant comprising a spinal implant. The spinal implant comprising a superior component and an inferior component.

In one embodiment, the method of deploying an implant using the implant deployment system comprises the steps of: selecting the proper or desired sized implant; assembling the implant deployment assembly, the implant deployment assembly comprising a draw bar, an actuation subassembly and a handle subassembly; loading or securing the implant to the implant deployment assembly to create the implant deployment system; inserting the implant system into a targeted anatomical area; attaching the screw guide onto the draw bar of the implant deployment assembly; inserting a fixation screw or fastener into the bore of the screw guide; securing the fixation screw to the implant at the targeted anatomical location by using the screwdriver; rotating a locking knob of the implant deployment assembly to allow the actuation subassembly to move or translate anteriorly relative to the draw bar to release the implant and deploy the implant into the targeted anatomical region. The implant comprising a spinal implant. The spinal implant comprising a superior component and an inferior component.

With reference to FIG. 33A, a surgeon may complete the step of assembling at least one spinal implant 3300 by assembling a selected superior component 3310 and an inferior component 3320 of the least one spinal implant 3300. In one disclosed embodiment, the at least one spinal implant may comprise a superior component 3310, an inferior component 3320, and a fixation screw (not shown). The at least one spinal implant may further comprise a retainer ring or retainer clip 3330. The superior component 3310 can comprise a superior articulation component 3340 which may comprise a socket surface having a concave shape. The inferior component comprises an inferior articulation component 3350 and a bridge 3360. The inferior articulation component may comprise a ball surface comprising a convex or hemi-spherical shape. The socket surface of the superior component desirably contacts and engages the ball surface of the inferior component to create a polyaxial articulation joint. The polyaxial articulation joint is desirably movable in a plurality of different orientations, which can including various combinations of flexion, extension, rotation, lateral flexion; contralateral flexion; and/or any combination thereof. The fixation screw is disposed through the posterior end of the bridge to be secured to inferior or caudal vertebral body. The fixation screw is disposed at an angle. The angle may match or substantially match the sagittal pedicle angle.

The superior component further comprises a superior base 3360 and a superior keel 3365. The superior articulation component is disposed onto the superior base and/or the superior articulation component is coupled to the superior base. The superior base comprises a first material and the superior articulation component can optionally comprise a second material. The first material may comprise the same material as the second material. The first material may comprise a different material than the second material. The materials may comprise a polymer, a metal and/or a ceramic. The polymer may comprise thermoplastics or thermosets. The polymer may further comprise cross-linked polymers. The polymer may comprise polyethylene (PE), high density polyethylene (HDPE), ultra-high molecular weight polyethylene (UHMWPE), and/or any combination thereof. The polymers may further comprise being cross-linked one or more times. The polymers may further comprise antioxidant doped or impregnated polymers. The antioxidants may include Vitamin E or Vitamin C. The metals may comprise stainless steel, titanium, titanium alloys, cobalt chrome, cobalt chrome alloys, and/or any combination thereof.

The inferior component 3320 further comprises an inferior base and an inferior keel 3375. The inferior articulation component is disposed onto the inferior base and/or the inferior articulation component is coupled to the inferior base. The bridge 3360 is coupled to the posterior end of the inferior base and extends posteriorly or extends in the posterior direction. The inferior base comprises a third material. Each of the first material, second material and/or third material may comprise the same material. Each of the first material, second material and/or the third material may comprise a different material. The materials may comprise a polymer, a metal and/or a ceramic. The polymer may comprise thermoplastics or thermosets. The polymer may further comprise cross-linked polymers. The polymer may comprise polyethylene (PE), high density polyethylene (HDPE), ultra-high molecular weight polyethylene (UHMWPE), and/or any combination thereof. The polymers may further comprise being cross-linked one or more times. The polymers may further comprise antioxidant doped or impregnated polymers. The antioxidants may include Vitamin E or Vitamin C. The metals may comprise stainless steel, titanium, titanium alloys, cobalt chrome, cobalt chrome alloys, and/or any combination thereof.

At least a portion of the bridge, the inferior base and/or the superior base may comprise a coating. Alternatively, the entirety of the bridge, inferior base and/or superior base may comprise a coating. The coatings may include inorganic coatings or organic coatings. The coatings may further include a metal coating, a polymer coating, a composite coating (ceramic-ceramic, polymer-ceramic, metal-ceramic, metal-metal, polymer-metal, etc.), a ceramic coating, an anti-microbial coating, a growth factor coating, a protein coating, a peptide coating, an anti-coagulant coating, an antioxidant coating and/or any combination thereof. The antioxidant coatings may comprise naturally occurring or synthetic compounds. The natural occurring compounds comprises Vitamin E and Vitamin C (tocotrienols and tocopherols, in general), phenolic compounds and carotenoids. Synthetic antioxidant compounds include a-lipoic acid, N-acetyl cysteine, melatonin, gallic acid, captopril, taurine, catechin, and quercetin, and/or any combination thereof. The coatings can be impregnated, applied and/or deposited using a variety of coating techniques. These techniques include sintered coating, electrophoretic coating, electrochemical, plasma spray, laser deposition, flame spray, biomimetic deposition and wet methods such as sol-gel-based spin-and-dip or spray-coating deposition have been used most often for coating implants.

The metal coatings may comprise titanium, titanium alloys, cobalt-chrome alloys, platinum and stainless steel, and/or any combination thereof. More specifically, the metal coating includes titanium and/or cobalt-chrome molybdenum (CoCrMo). The polymer coatings may include thermoplastic or thermoset polymers. The polymers may further include carbon fiber, polyether ether ketone (PEEK), polyethylene (PE), ultra-high molecular weight polyethylene (UHMWPE), polycarbonate (PC), polypropylene (PP) and/or any combination thereof. The ceramic coatings may include alumina ceramics, Zirconia (ZrO2) ceramics, Calcium phosphate or hydroxyapatite (Ca10(PO46(OH)2) ceramics, titanium dioxide (TiO2), silica (SiO2), Zinc Oxide (ZnO) and/or any combination thereof.

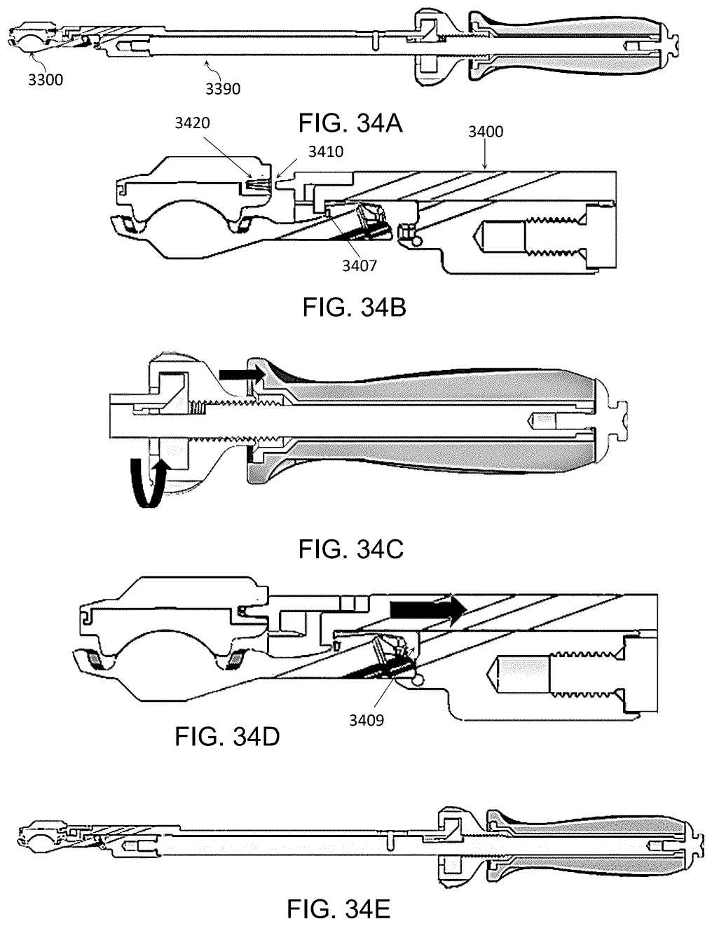

With reference to FIGS. 33B-33D and FIGS. 34A-34D, the surgeon may complete the step of securing the at least one spinal implant 3300, a first spinal implant and/or a second spinal implant to one or more deployment tools 3390. The surgeon may tilt the first end of the assembled deployment tool and insert the posterior end or second end of the implant into the pocket of the grasping tip disposed on the first end of the deployment tool. The surgeon can secure the posterior end or second end of the spinal implant into the hook of the grasping tip disposed on the first end of the deployment tool. More specifically, the surgeon can insert the hook of the grasping tip to contact or engage with the retainer clip channel on the posterior end of the bridge of the spinal implant. The locking knob should be rotated (e.g., clockwise or counterclockwise—see FIG. 34D) to translate the at least one spinal implant posteriorly until at least a portion of the at least one spinal implant contacts and/or engages with the stop wall 3409 of the grasping tip and the protrusion 3410 on the draw bar 3400 is inserted into the protrusion socket 3420 or recess, as best seen in FIG. 34B. The protrusion socket or recess is sized and configured to receive at least a portion of the protrusion of the locking arm. The surgeon will desirably experience some force feedback on the locking knob to indicate that contact has been made against the stop wall of the grasping tip. The at least one spinal implant should be in a neutral position and/or aligned with the longitudinal axis of the deployment tool. The at least one spinal implant should be in a neutral position and/or parallel with the longitudinal axis of the deployment tool.

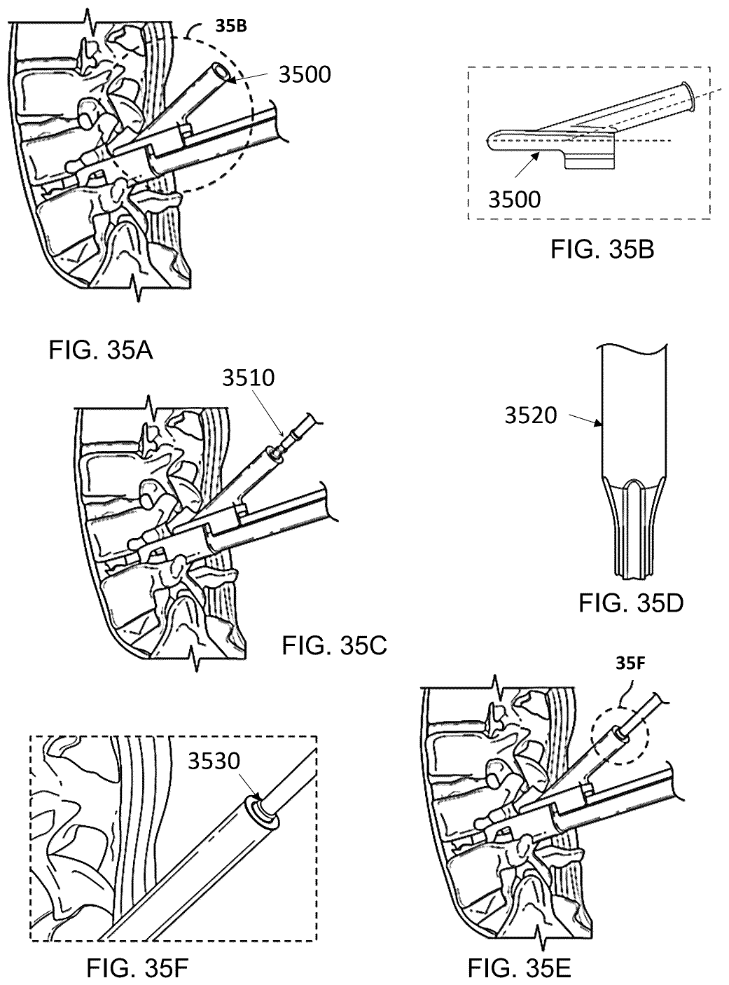

With reference to FIGS. 35A-35E, the surgeon may complete the steps of deploying the at least one spinal implant into the at least one side. The step of deploying the at least one spinal implant into the at least one side comprises the steps of: aligning the each of the superior and/or inferior keels on the spinal implant to each of the caudal and/or cranial keel channels on at least one side; securing fixation screw into the caudal or inferior vertebral body into the at least one side; and releasing or disengaging the deployment tool on the at least one side.

With reference to FIG. 35A, the surgeon may complete the step of aligning each of the superior and/or inferior keels on the spinal implant to each of the caudal and/or cranial keel channels on at least one side, a first side and/or a second side. The surgeon should align the superior keel of the at least one spinal implant with the cranial keel channel and the inferior keel of the at least one spinal implant with the caudal keel. The surgeon can then translate and/or push the deployment tool forward until the superior keel and/or the inferior keel of the at least one spinal implant contacts and/or engages with at least one anterior end or anterior surface of the caudal and/or cranial keel channels. The pushing force should be a minimal force. The surgeon should be aware to not over distract prepared intravertebral disc space that what was originally pre-determined during the step of selecting a proper spinal implant size.

With reference to 35B-35F, the surgeon may complete the step of securing the fixation screw to the caudal or inferior vertebral body. The surgeon may desirably attach a screw guide 3500 near and/or proximate to medial and/or lateral rails at the first end of the deployment tool (FIGS. 35A and 35B). The screw guide will be positioned and/or disposed onto a portion of the grasping tip and/or a portion to the shaft subassembly and secured. Alternatively, the screw guide may be attached prior to the step of aligning each of the superior and/or inferior keels to each of the caudal and/or cranial keel channels. The guide barrel or the guide tube of the screw guide can be positioned obliquely and/or at an angle relative the longitudinal axis of the body of the screw guide. Accordingly, the longitudinal axis of the guide barrel or the guide tube is at an angle relative to the longitudinal axis of the body of the screw guide. The surgeon may desirably insert the fixation screw 3510 into the bore (e.g., inner diameter) of the guide barrel or guide tube of the screw guide. A deployment screwdriver 3520 may be utilized to secure the fixation screw into the caudal vertebral body. The deployment screwdriver may be powered and/or manual. The deployment screwdriver is desirably coupled to a quick-connect handle (e.g., a Hudson handle) for manual installation. The surgeon will insert the drive tip or tip into a portion of the drive recess of the fixation screw. The surgeon may begin to rotate the deployment screwdriver clockwise to perforate the cortex using the fixation screw tip until the surgeon receives feedback and/or torque feedback (e.g., tightening). The surgeon should not over-rotate, over-tighten or over-torque the fixation screw into the vertebral body—it may cause changes to positioning, alignment, and/or prepared bone interface.

With reference to FIG. 35E, the surgeon may complete the step of disengaging, disconnecting and/or releasing the at least on spinal implant deployed into the at least one side. Once the fixation screw is secured into the caudal vertebral body, the deployment screwdriver may be removed. The surgeon may slightly release the deployment tool securing force from the at least one spinal implant by turning and/or rotating the locking knob (e.g., counterclockwise or clockwise) of the deployment tool. The deployment screwdriver should be rotated (e.g., counterclockwise or clockwise) until the reference line 3530 on the deployment screwdriver is visible above a top surface of the guide barrel and/or the guide tube. Disconnect and/or disengage the deployment tool from the at least one spinal implant and remove from the prepared intravertebral space.

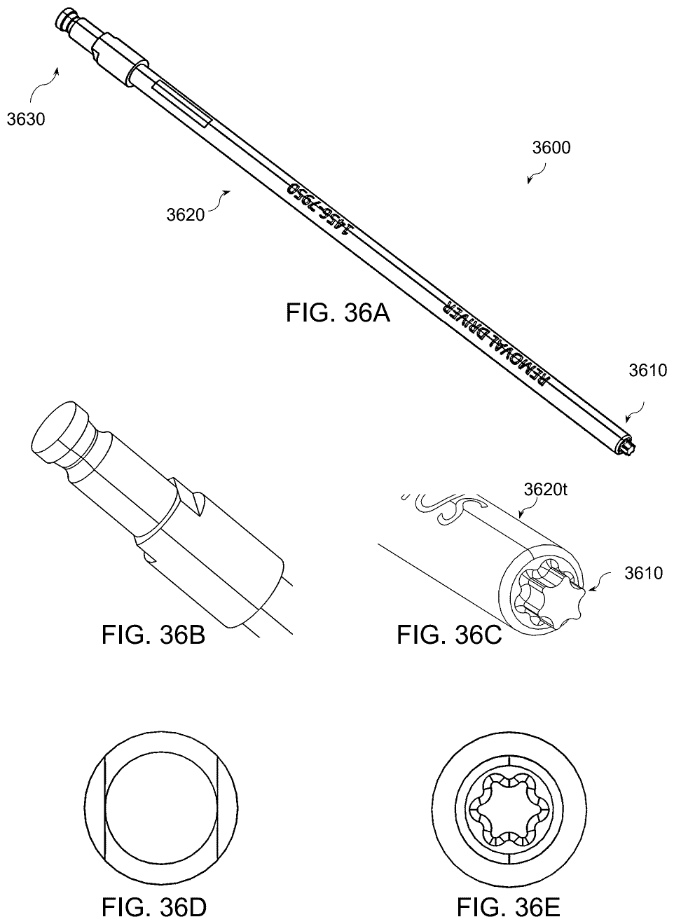

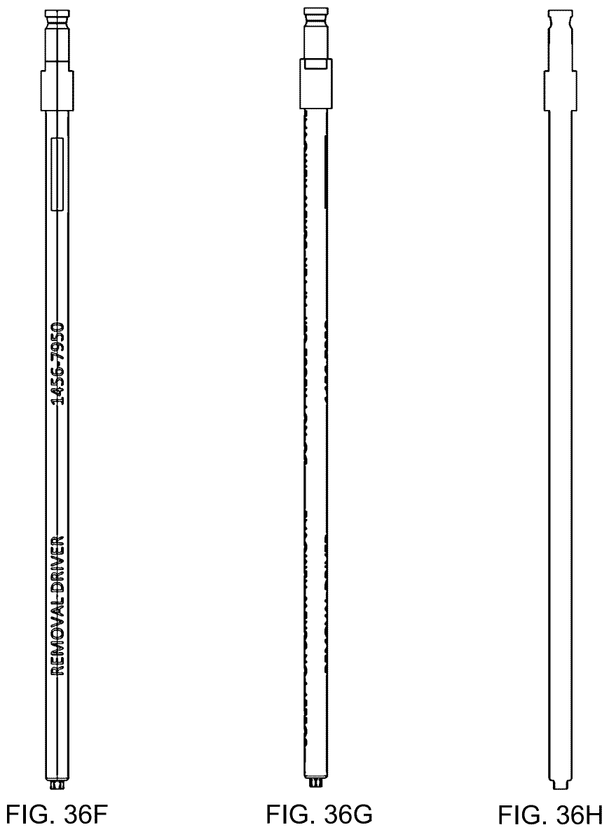

FIGS. 36A-36H, 37A-37I, 38A-38H, 39, 40A-40F, 41A-41D, 42A-42J and 43A-43B, depict various plan views and cross-sectional views of one embodiment of a removal system. The removal system is one or tools or components that help aid the surgeon or user to remove the one or more implants from the targeted intervertebral space in cases of implant misalignment, improper sizing, improper bony preparation, and/or any other reasons. The implant removal system comprises a removal tool and a removal driver. The implant removal system may further comprise a slap hammer.

FIGS. 36A-36H depicts various views and cross-sectional views of one embodiment of a removal driver 3600. The removal driver tool allows the surgeon or user to remove fixation screws or fasteners. The removal driver tool tip 3610 engages with the driving head of a fixation screw or fastener to allow the surgeon or user to provide a turning force or torque in a counterclockwise direction for removal of the fixation screw and prepare the one or more implants for removal. The removal driver comprises a driving tip 3610, a shaft 3620 and a connector 3630. The removal driver further comprises a longitudinal axis, a first end and a second end.

The driving tip 3610 of the removal driver comprises a tip driving type and/or a total tip length. The driving tip is disposed at the first end of the screwdriver. The total tip length of the driving tip comprises 2 mm to 10 mm length; the total length of the driving tip comprises 2 mm to 5 mm; and/or the total length of the driving tip comprises 2 mm to 3 mm; and/or the total length of the driving tip comprises greater than 2 mm. The driving tip can be straight. The driving tip is disposed and engaged within the driving recess of the head of the fixation screw or fastener. The driving tip comprises a driving type, the driving type includes a flathead, a Phillips, a Pozidrive, a Hex or Hex Key or Allen, a Torx, square head, a Tri-point and/or any combination thereof. In one embodiment, the driving type comprises a hex or hex key or hexalobular type shape.

The screwdriver may comprise a shaft. The shaft comprises a first end, a second end, a length, and/or a diameter. The diameter of the shaft may comprise a range of 5 mm to 8 mm; the diameter may further comprise a range of 6 mm to 8 mm; the diameter may further comprise a range of 5 mm to 6 mm. The diameter of the shaft may be uniform along the longitudinal axis and/or length of the shaft. The shaft may comprise a shape, the shape may be cylindrical or generally cylindrical. The shape may comprise a cylindrical or generally cylindrical shape without a tapered transition. The length of the shaft may comprise a range of 125 mm to 175 mm; the length of the shaft may comprise a range of 140 mm to 165 mm; the length of the shaft may comprise a range of 158 mm to 162 mm; and the length of the shaft may be greater than 155 mm. The driving tip is disposed at a first end, and the connector disposed at a second end. The shaft may be solid or hollow.

In one embodiment, the removal driver does not comprise a transition between the shaft and the driving tip (e.g., no tapering). The diameter of the shaft is larger than a diameter of the driving tip to create a shoulder. While removal driver is inserted into the one or more implants to engage the one or more fixation screws, the shoulder of the removal driver shaft allows the removal driver to push through the spring-loaded retainer clip of an implant, and separate arms of the spring-loaded retainer clip and maintain it open until removal of the fastener is completed. Once the user or surgeon removes the removal driver the spring-loaded retainer clip returns to its original position.

The removal driver may comprise a connector. The connector may include a male and/or a female connector. The connectors may be configured to engage with a Hudson connection, an Association for Osteosynthesis (AO) connection and/or a Trinkle connection. The connection mechanisms comprise quick-connect and quick-disconnect. The connector may be disposed within and/or coupled to a manual handle and/or a powered handle or handpiece.

The removal driver and/or removal driver shaft may further comprise one or more reference markers. The reference markers are used when the removal driver is inserted into the intervertebral disc space to help guide the surgeon and/or user on positioning of the removal driver tool relative to the intervertebral disc space. The one or more reference markers may be confirmed radiographically to determine the length of insertion of the removal driver into the intervertebral disc space to release the fixation from the targeted anatomical region. The one or more reference markers may comprise a radiopaque material or be radiopaque to allow visualization with an imaging equipment. The one or more reference markers may comprise a laser marking, a ring, a channel, and/or an opening. The one or more reference markers may be disposed onto a portion of the head and/or the shaft.

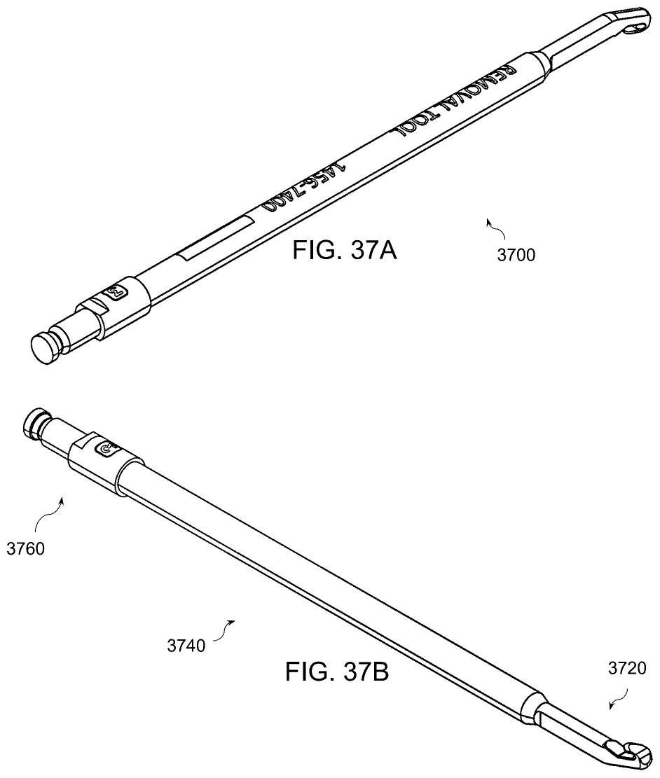

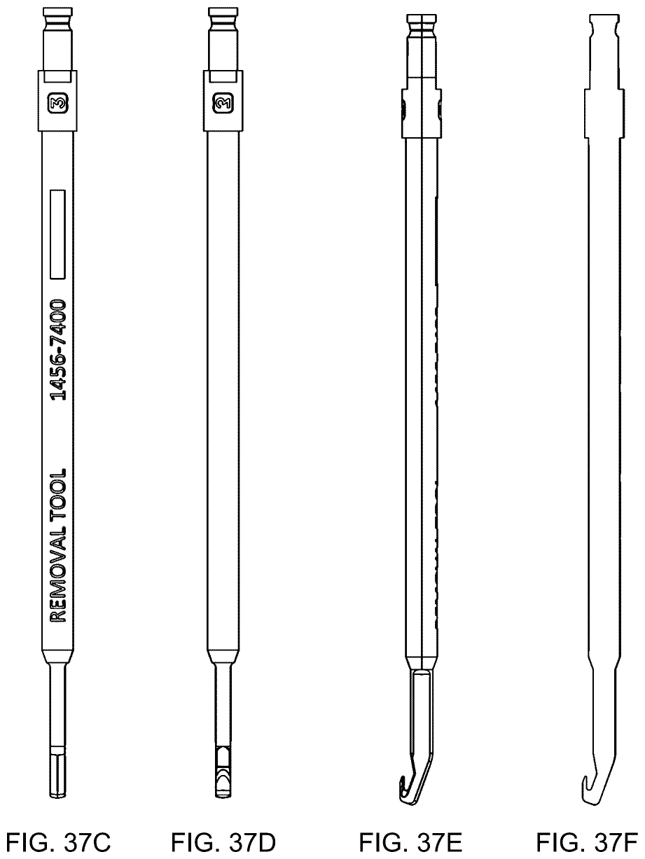

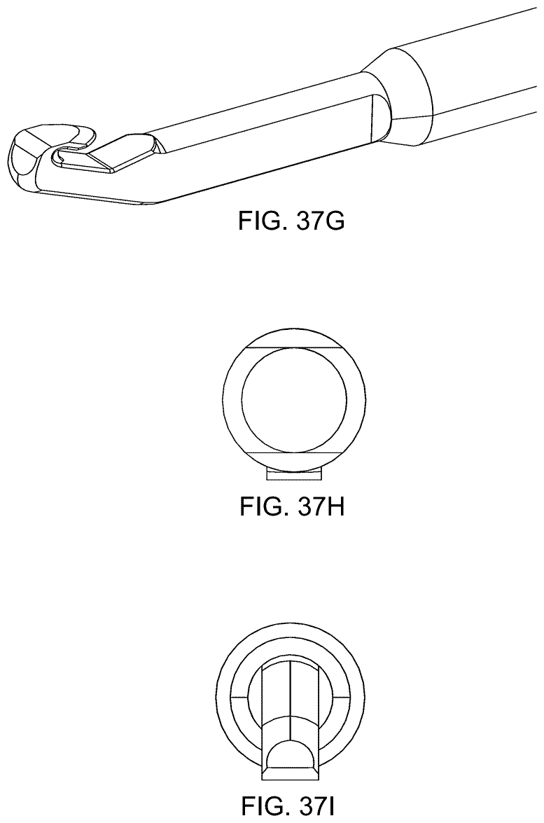

FIGS. 37A-37I depict various plan view and a cross-sectional view of one embodiment of a removal hook 3700 or removal tool of the removal system. The removal hook 3700 is a component or tool that is used to remove one or more implants deployed into one or more targeted anatomical areas. The removal hook engages with a portion of the implant to allow the surgeon and/or user to pull the implant posteriorly until it is out of the targeted anatomical area. The removal hook 3700 can comprise a hook tip 3720, a shaft 3740 and a connector 3760.

The hook tip of the removal hook tool comprises a hook and/or a total tip length. The hook tip is disposed at the first end of the removal hook tool. The total tip length of the hook tip comprises 20 mm to 35 mm length; the total length of the hook tip comprises 23 mm to 30 mm; and/or the total length of the driving tip comprises 24 mm to 26 mm; and/or the total length of the driving tip comprises greater than 23 mm. The hook tip comprises a first portion and a second portion. The first portion comprises a first portion width and a first portion length. The first portion length comprises 5 mm to 10 mm; the first portion length comprises 6 mm to 8 mm; and/or the first portion length is equal to and greater than 5 mm. The first portion comprises a shape, the shape includes a “U” shape. The first portion comprises an angled orientation relative to the longitudinal axis of the hook removal tool. The angled orientation comprises 15 degrees to 25 degrees; the angled orientation comprises 18 degrees to 22 degrees; and/or the angled orientation comprises equal to and greater than 18 degrees.

The second portion comprises a second portion width and/or second portion length. The second portion length comprises 10 mm to 20 mm; the second portion length is approximately 15 mm to 18 mm; the second portion length is approximately equal to and greater than 15 mm. The second portion further comprising a shape, the shape is substantially square, rectangular, square-round and/or rectangular round. The second portion is straight and extends co-axially with the longitudinal axis of the removal hook tool. A tapered transition is disposed between the first end of the shaft and the second portion of the hook tip. The second portion width is the same as the first portion width of the hook tip. The second portion width is different than the first portion width of the hook tip. The total tip length comprises 20 mm to 30 mm; the total tip length comprises 23 mm to 26 mm; and/or the total tip length comprises equal to or greater than 23 mm. The hook tip may comprise a hollow or solid tip.

The removal hook tool may comprise a shaft. The shaft comprises a first end, a second end, a length, and/or a diameter. The diameter of the shaft may comprise a range of 5 mm to 8 mm; the diameter may further comprise a range of 6 mm to 8 mm; the diameter may further comprise a range of 6 mm to 7 mm. The diameter of the shaft may be uniform along the longitudinal axis and/or length of the shaft. The shaft may comprise a shape, the shape may be cylindrical or generally cylindrical. The shape may comprise a cylindrical or generally cylindrical shape without a tapered transition disposed at a first or second end. The length of the shaft may comprise a range of 100 mm to 125 mm; the length of the shaft may comprise a range of 105 mm to 115 mm; the length of the shaft may comprise a range of 105 mm to 110 mm; and the length of the shaft may be greater than 105 mm. The hook tip is disposed at a first end, and the connector disposed at a second end. The shaft may be solid or hollow.

The removal hook tool may comprise a connector. The connector may include a male and/or a female connector. The connectors may be configured to engage with a Hudson connection, an Association for Osteosynthesis (AO) connection and/or a Trinkle connection. The connection mechanisms comprise quick-connect and quick-disconnect. The connector may be disposed within and/or coupled to a manual handle and/or a powered handle or handpiece.

The removal hook tool and/or removal hook shaft may further comprise one or more reference markers. The reference markers are used when the removal hook tool is inserted into the intervertebral disc space to help guide the surgeon and/or user on positioning of the removal hook tool relative to the intervertebral disc space. The one or more reference markers may be confirmed radiographically to determine the length of insertion of the removal driver into the intervertebral disc space to release the fixation from the targeted anatomical region. The one or more reference markers may comprise a radiopaque material or be radiopaque to allow visualization with an imaging equipment. The one or more reference markers may comprise a laser marking, a ring, a channel, and/or an opening. The one or more reference markers may be disposed onto a portion of the head and/or the shaft.

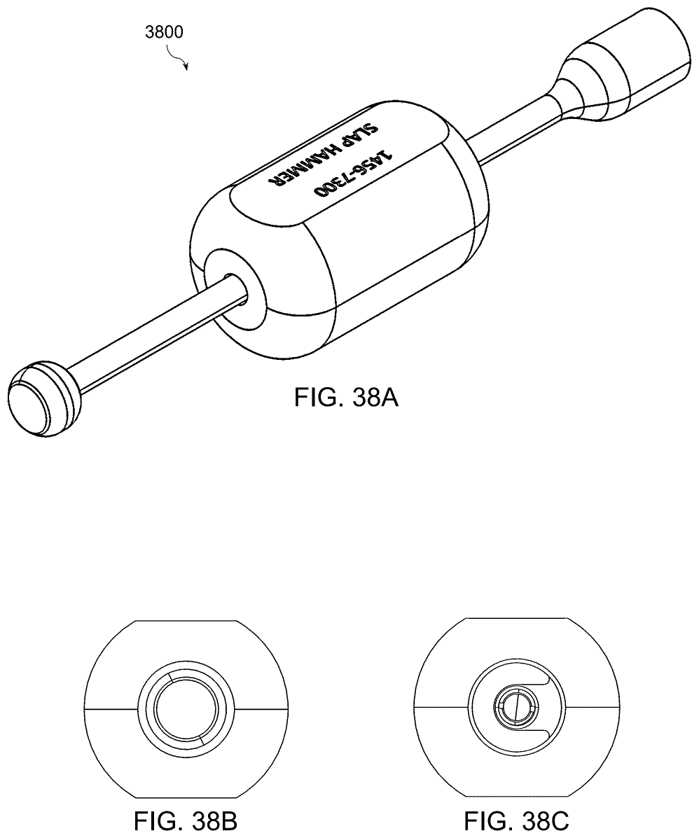

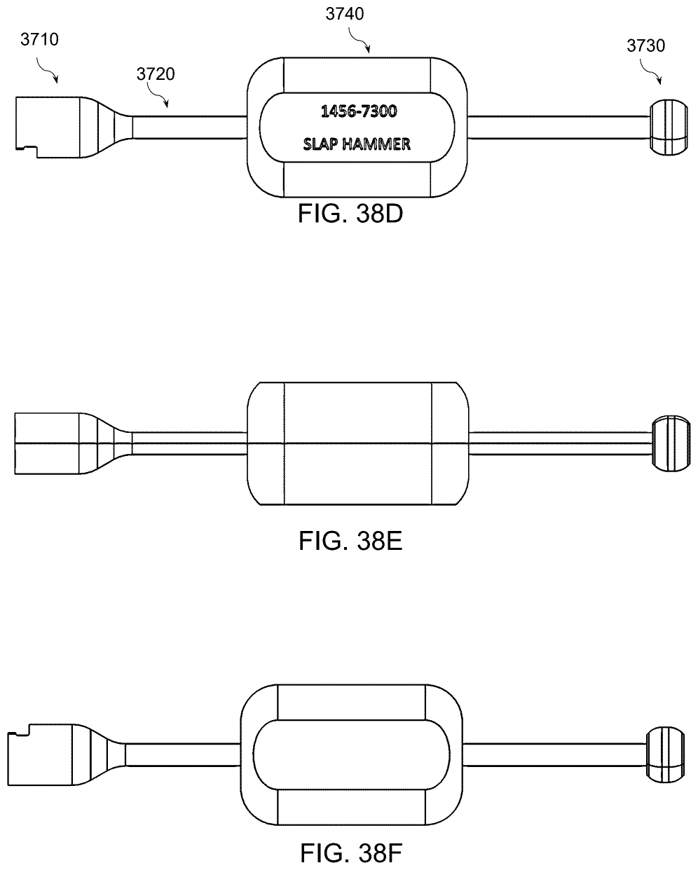

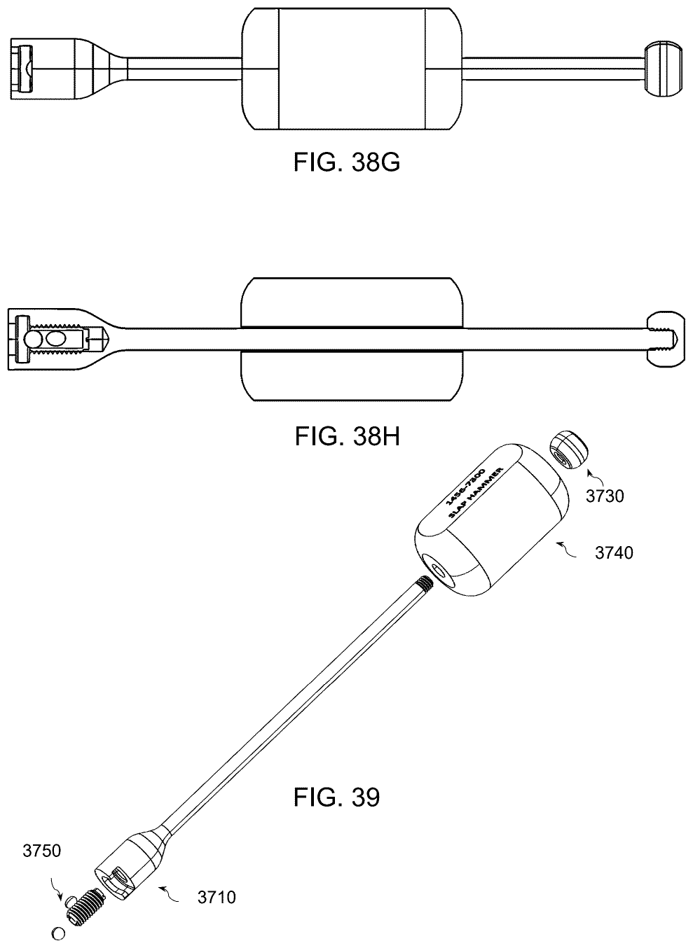

FIGS. 38A-38H, 39, 40A-40F, 41A-41D and 42A-42J depict various plan views, an exploded view and cross-sectional views of one embodiment of a slap hammer assembly 3800. The slap hammer assembly is a tool that can be safely and easily manipulated by a user or surgeon for applying a desired amount of force to a targeted object by using a sliding weight that translates along an axis to create a “jerking” force without having to substantially change the user's physical efforts in manipulating the tool. More specifically, the slap hammer assembly can comprise a guide shaft 3720 and a sliding weight 3740. One end of the guide rod is affixed to an object or surface at a connection point 3710 (which may optionally include a retention detent 3750 or similar device), such as to a surgical implement. The sliding weight may be thrown towards a stop 3730, generating a jerking force when the sliding weight strikes the stop 3730 on the end of the guide rod in a known manner. The sliding weight may be repeatedly “thrown” to extract a surgical instrument and/or one or more implants. Alternatively, when the slap hammer assembly is affixed to a loose object or surface, the hammer may be dropped or thrown downward toward the object for a precision impact. The slap hammer assembly comprises guide rod, a weight or hammer, and a stop. The slap hammer assembly may further comprise a connection mechanism. The slap hammer assembly further comprising a striking component.

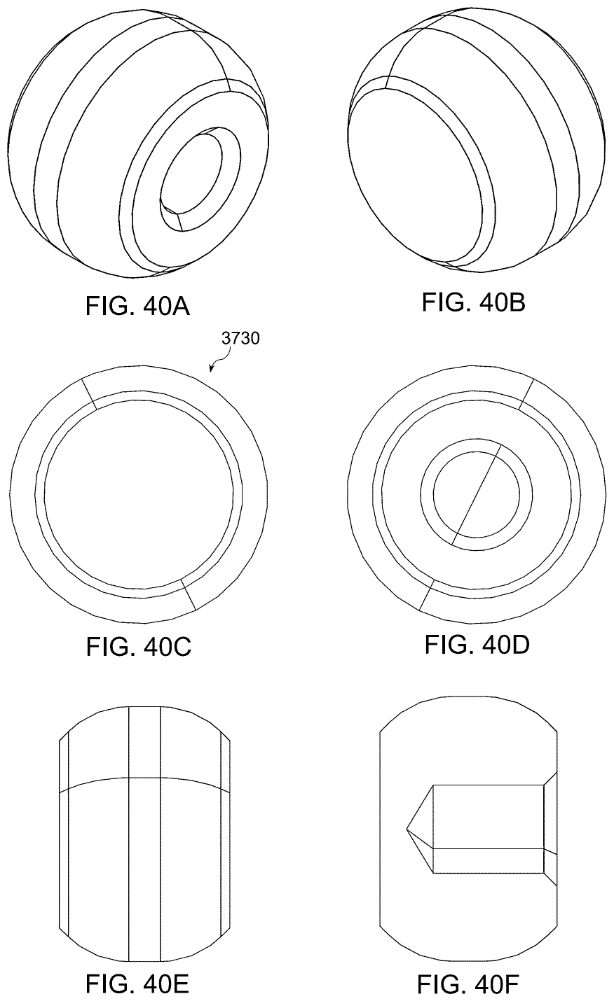

FIGS. 40A-40F depicts various plan views and a cross sectional view of one embodiment of a stop 3730 of the slap hammer assembly. The stop comprising an outer surface or outer diameter, an inner diameter, inner surface or bore, a top surface, and a bottom surface. The stop further comprising a bore axis and a shape. The bore extends from a bottom surface of the stop towards the top surface. The bore is sized and configured to receive a portion of the retention mechanism. The bore further comprising threads. The bore axis coaxially aligned with a longitudinal axis of a guide shaft. The shape comprises a sphere or ball. The outer diameter of the stop is larger than the outer diameter of the guide shaft. At least a portion of the bottom surface of the stop is flat or planar.

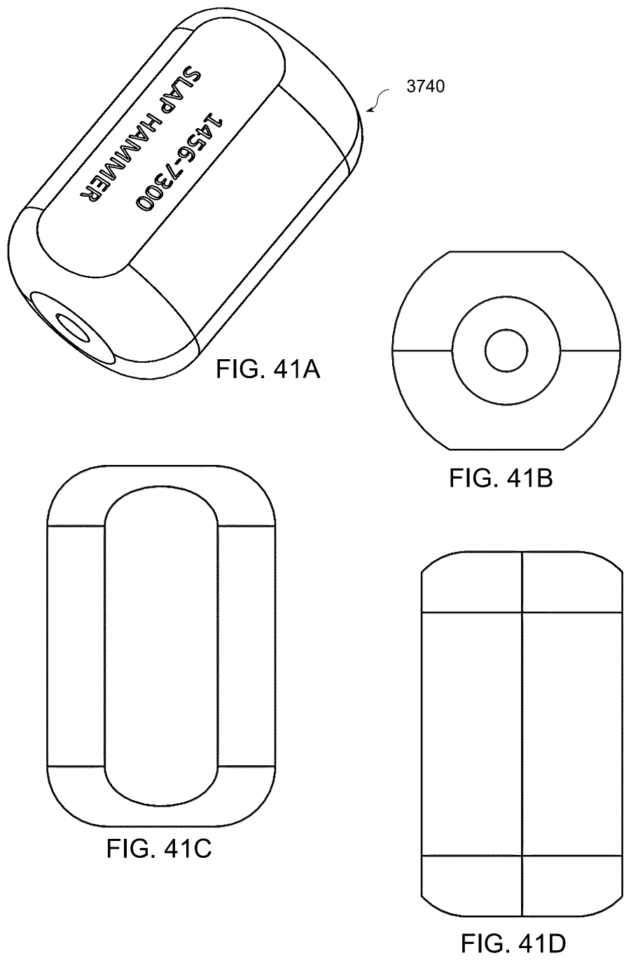

FIGS. 41A-41D depicts various plan views of one embodiment of a hammer 3740 or weight of the slap hammer assembly. The hammer or weight comprises a hammer longitudinal axis, a lumen, an outer diameter, a first surface and a second surface. The lumen extends from the first surface through the second surface. The lumen extends along at least a portion of a length of the hammer. The lumen extends along the longitudinal axis of the hammer. The lumen further comprises a lumen inner diameter. The lumen inner diameter is larger than the guide shaft outer diameter. At least a portion of the first surface and a second surface comprises a flat or planar surface. At least a portion of the first and/or second surface contacts or engages with the bottom surface of the stop during the sliding action of the hammer. The hammer further comprises an outer surface or outer diameter. The outer surface of the hammer further comprises one or more flat surfaces. Each of the one or more flat surfaces are radially spaced apart, the spacing may comprise 90 degrees or 180 degrees apart.

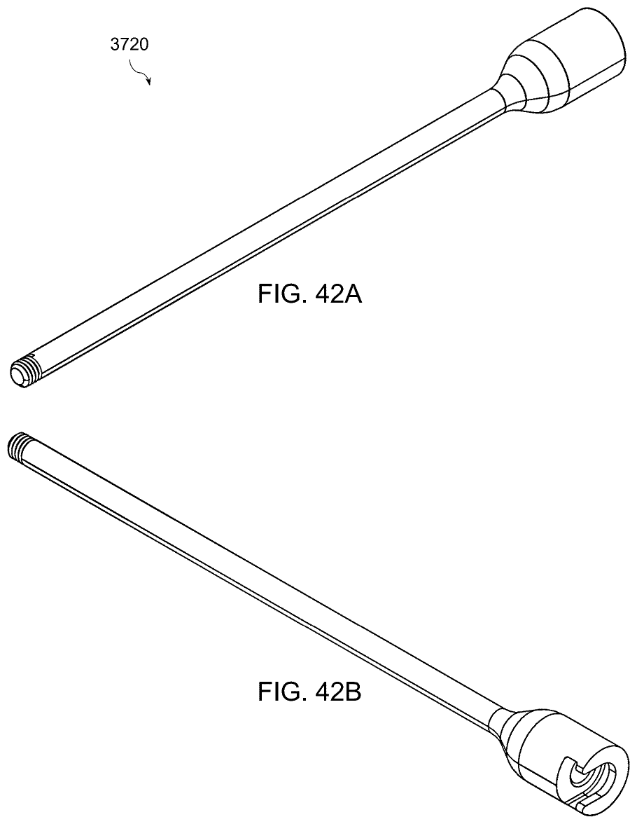

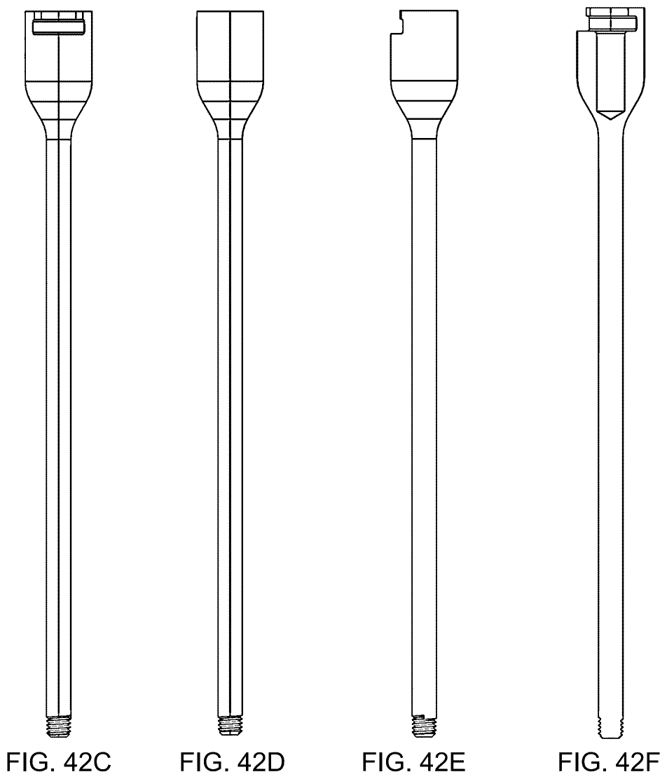

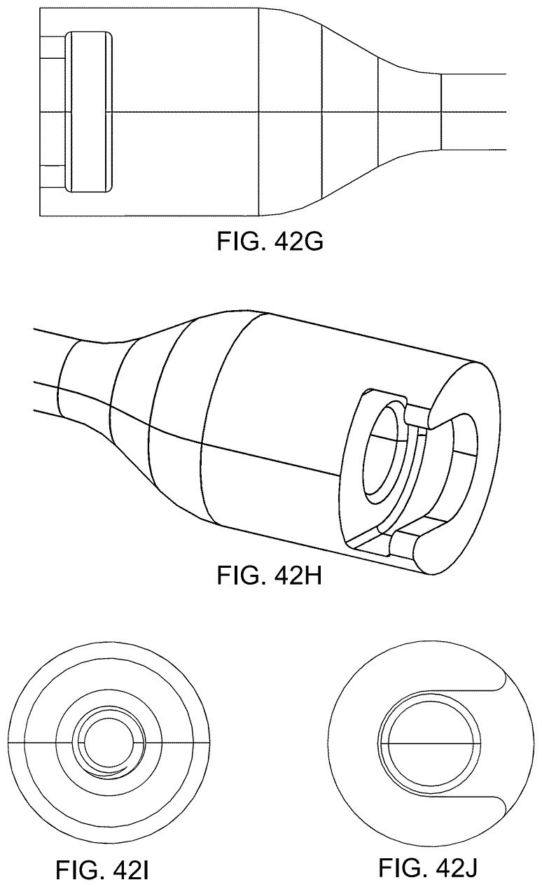

FIGS. 42A-42J depicts various plan views and a cross-sectional view of one embodiment of a guide shaft 3720. The guide shaft further comprises an outer diameter, a first end, a second end, and a longitudinal axis. The guide shaft further comprises a shaft, a striking component and a threads. The threads are disposed at least a portion on the second end of the guide shaft. The threads comprise external threads disposed on the outer diameter of the guide shaft. The threads are sized a configured to be disposed with the bore or threaded bore of the stop to couple the guide shaft to the stop. The external threads of the guide shaft contact and engage the threads of the bore to lock, couple or secure the guide shaft to the stop. The shaft comprising an outer diameter. At least a portion of the outer diameter is sized and configured to be disposed within the lumen of the hammer. At least a portion of the shaft is disposed within the lumen and extends through the lumen to be coupled to the stop. The shaft further comprises a shape, the shape is cylindrical or generally cylindrical. The shaft may be solid or hollow.

The striking component is disposed at a first end. The striking component comprises a groove, channel or recess and a bore. The bore comprising a bore axis. The bore extending from a first end or a top surface towards the second end or the threaded second end. The bore is sized and configured to receive a portion of the retention mechanism. The bore may comprise threads. The bore axis of the striking component may be aligned or co-axial with the longitudinal axis of the guide shaft. The recess, groove or channel may further comprise a top surface and a bottom surface, and recess or groove width. The recess, groove or channel width is sized and configured to receive a portion of an element. The element may comprise a portion of surgical instrument or a portion of an implant. The portion of the surgical instrument may further comprise a portion of a connector of a surgical instrument. The connector may comprise an AO connector or a Hudson connector.

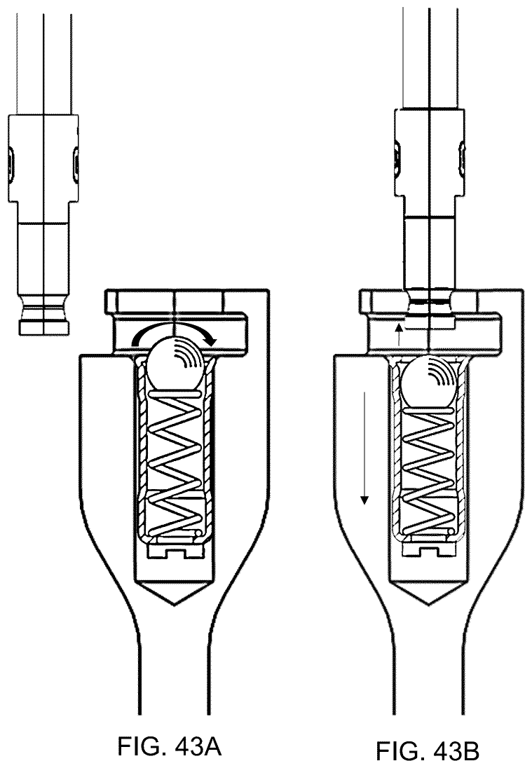

FIGS. 43A-43B depict a cross sectional view of one embodiment of a retention mechanism. The retention mechanism may comprise a spring, a ball plunger, and/or a fastener. The retention mechanism is removably connected from the striking component and/or to the element. In one embodiment, the retention mechanism comprises a ball plunger. This type of use for a ball plunger is to lock something into position and provide some dampening. The retention mechanism further comprising an outer diameter or outer surface, the outer diameter or outer surface being sized and configured to be disposed within the bore of the striking component.

The ball plunger comprises a detent ball, a threaded body, an compression spring. As at least a portion of the element is slid within the recess, groove or channel, the at least a portion of the element contacts the ball detent to place on the outer surface. The contact force on the ball detent outer surface puts pressure on the plunger or nose of the spring plunger, the compression spring depresses into the threaded body forcing the compression spring to compress allowing the contact force to position the nose into the desired notched out area. However, at the same time, the compression spring provides an upward force while the ball detent contacts a portion of a bottom surface of the element driving at least a portion of the element to contact the upper surface of the recess, groove or channel to lock into position. The threaded body is disposed with the threads of the bore of the striking component to releasably lock the retention mechanism to the guide shaft.

The removal driver, the removal hook tool, and each of the components of the slap hammer assembly further comprise a material. The material includes a metal, a polymer, and/or ceramic. The material may include a polymer, a metal and/or a ceramic. The polymer may include a thermoset or thermoplastic. The metal may include stainless steel, a stainless-steel alloy, titanium, titanium alloy, cobalt chrome, cobalt chrome alloy, nitinol, and/or tantalum. The removal driver, the removal hook tool, and each of the components of the slap hammer assembly (e.g., the stop, the shaft, the striking component, and/or the hammer, etc.) further comprises a surface texture. The surface texture comprises a rough or polished texture. The roughened surfaces or porous surfaces, including turned, blasting, sand blasting, acid etching, chemical etching, dual acid etched, plasma sprayed, anodized surfaces, and/or any combination thereof. The polished surface may be accomplished different techniques, mechanical polishing, chemical polishing, electrolytic polishing, and/or any combination thereof. The polished surfaces can be measured in “Ra” micrometers (μm) or microinches (μin.). The Ra may comprise a range of 0.025 to 1.60 μm; may comprise a range of 0.025 to 0.40 μm; and/or may comprise a range of 0.20 to 0.40 μm. Accordingly, the Ra may comprise

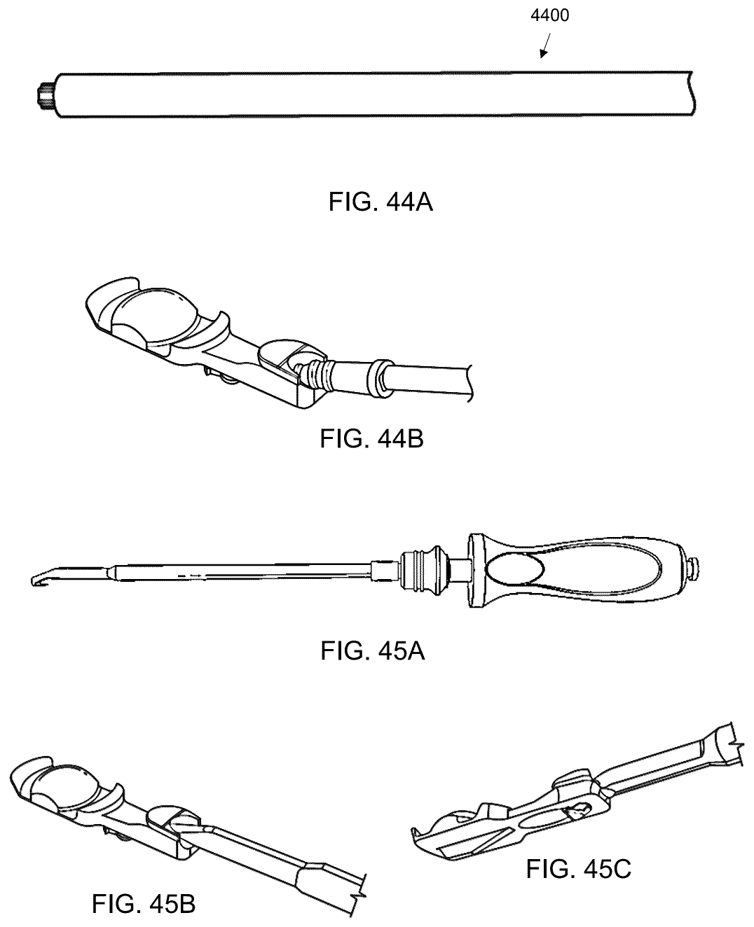

FIGS. 44A-44B and 45A-45C depict one embodiment of a method of using the removal system. The method of using the removal system comprises the steps of: inserting a removal driver 4400 into the intervertebral space or targeted anatomical region until the removal driver contacts the head of the fixation screw of an implant; removing the fixation screw from the implant and out of the intervertebral space or targeted anatomical area; inserting the removal hook tool into the intervertebral space or the targeted anatomical region until a portion of the hook tip of the removal hook tool contacts or engages a portion of the implant; removing at least a portion of the implant by pulling the removal hook tool and out of the intervertebral space or targeted anatomical area.

In another embodiment, the method of using the removal system comprises the steps of: inserting the removal driver into the intervertebral space or targeted anatomical region until the removal driver contacts the head of the fixation screw of an implant; removing the fixation screw from the implant and out of the intervertebral space or targeted anatomical area; inserting the removal hook tool into the intervertebral space or the targeted anatomical region until a portion of the hook tip of the removal hook tool contacts or engages a portion of the implant; coupling the first end of the slap hammer assembly to a portion of the removal hook tool second end, the slap hammer assembly comprising a stop, a guide shaft, and a striking component; sliding the hammer forcibly along a guide shaft between the stop and the striking component of the slap hammer assembly; and removing at least a portion of the implant out of the intervertebral space or targeted anatomical area.

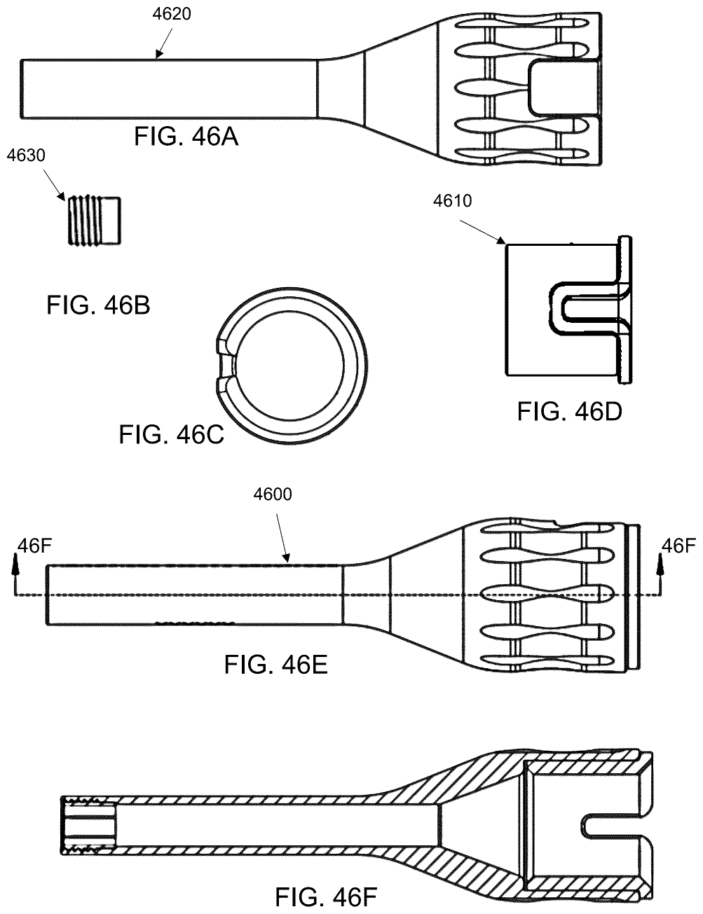

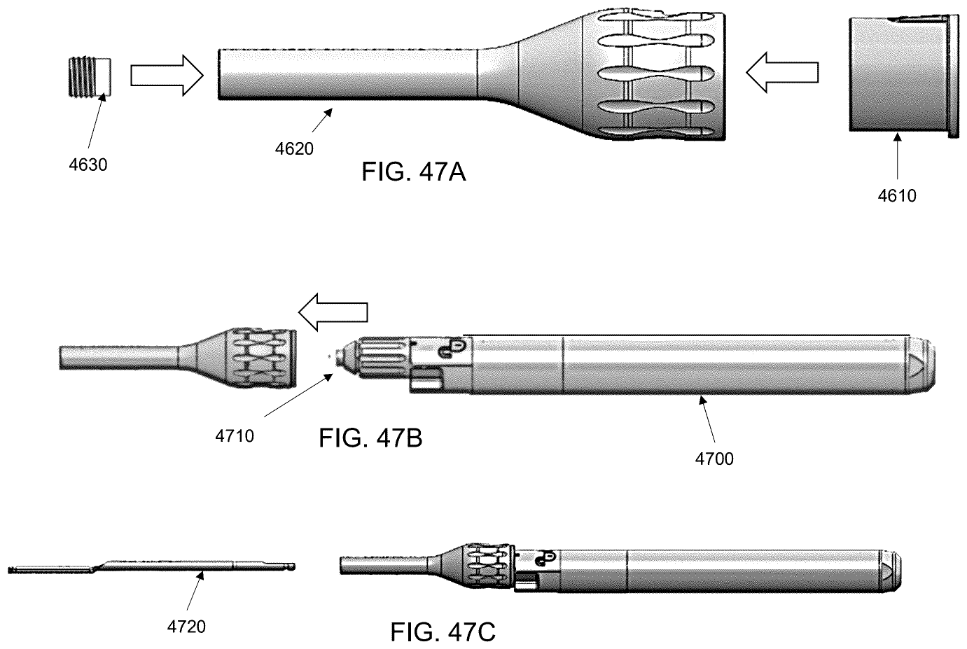

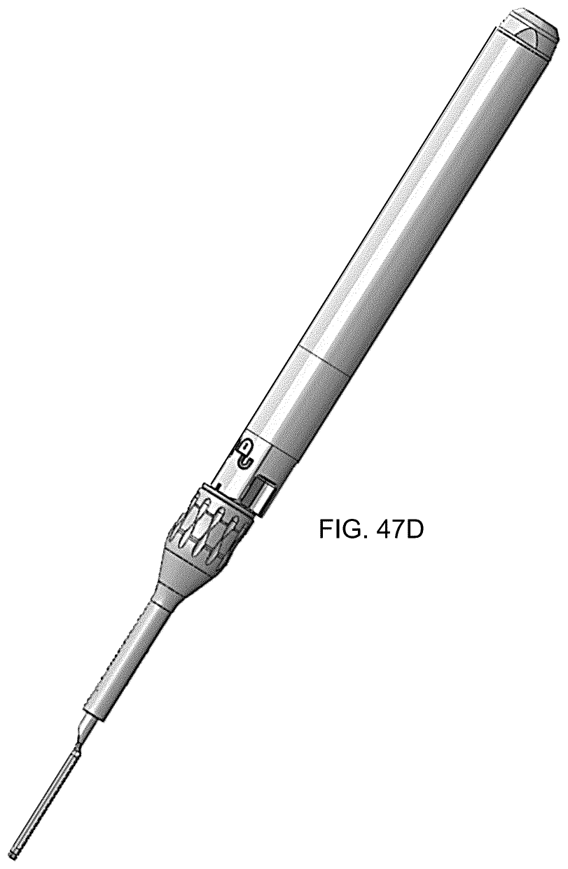

FIGS. 46A through 46F depict an exemplary embodiment of an optional protection sleeve 4600 for a powered rasping tool. Where a surgeon wishes to utilize a smaller and/or reduced size incision (or various minimally invasive techniques), it may be desirous to protect surrounding tissue structures from contact with the oscillating rasp shaft. The sleeve can comprise a base plug 4610, a cylindrical body 4620 and a tip plug 4630, which are assembled together as depicted in FIGS. 46E and 47A. The base plug of the sleeve 4600 is then inserted over and/or engages with a tip 4710 of a handpiece 4700 (See FIG. 47B), and a rasp 4720 is then inserted into the tip plug of the sleeve and then advanced into the handpiece in the normal manner (See 47D). In this way, the outer surface of the sleeve, which does not move during operation of the rasp, can contact and sit safely against tissues without causing injury or abrasion to such structures.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the descriptions provided herein.

Many of the aspects and advantages of the present invention may be more clearly understood and appreciated by reference to the accompanying drawings. The accompanying drawings are incorporated herein and form a part of the specification, illustrating embodiments of the present invention and together with the description, disclose the principles of the invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein. What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

I/We claim:

1. A keel alignment system comprising:
a handle;
a fixed keel rasp disposed substantially in a first orientation or plane, the fixed keel rasp comprising a keel rasp head, the keel rasp head comprising a first one or more protrusions, the first one or more protrusions extending towards the superior direction; and
a movable guide tool assembly disposed substantially in a second orientation or plane, the movable guide arm removably coupled to the handle, the movable guide tool assembly comprising a guide arm, the guide arm including a guide head and a hand grip, the guide head comprising a second one or more protrusions, the second one or more protrusions extending towards the inferior direction,
the movable guide tool assembly moves relative to the fixed keel rasp from the second orientation towards the first orientation until a portion of the hand grip contacts or engages at least a portion of the handle when compressing or squeezing a portion of hand grip of the movable guide tool assembly to create a spacing between the guide head and the keel rasp head.

2. The keel alignment system of claim 1, wherein the handle comprises a powered handle or a manual handle.

3. The keel alignment system of claim 1, wherein the first one or more protrusions of the fixed keel rasp is coaxially aligned with the second one or more protrusions of the movable guide tool.

4. The keel alignment system of claim 1, wherein keel rasp head is parallel relative to the guide head and the first one or more protrusions of the keel rasp head is coaxially aligned with the second one or more protrusions of the guide head.

5. The keel alignment system of claim 1, wherein the first one or more protrusions comprise a plurality of sharp teeth.

6. The keel alignment system of claim 1, wherein the first one or more protrusions comprise a plurality of sharp teeth and the second one or more protrusions comprise a plurality of flat and smooth surfaces.

7. The keel alignment system of claim 1, wherein the second one or more protrusions of the guide head of the movable guide assembly is sized and configured to be disposed into a keel channel.

8. The keel alignment system of claim 1, wherein the guide head comprises a first contacting surface and a second contact surface, at least a portion of the first contact surface may contact or engage a bone surface.

9. The keel alignment system of claim 1, wherein the guide head comprises a first contacting surface and a second contact surface, at least a portion of the first contact surface may contact or engage a bone surface.

10. A keel alignment system comprising:
a handle;
a keel rasp disposed in a first plane, the keel rasp comprising a keel rasp head, the keel rasp head comprising a first one or more protrusions, the first one or more protrusions extending towards the superior direction, the keel rasp is removably coupled to the handle; and
a guide tool assembly disposed in a second plane, the movable guide tool assembly comprising a guide arm, the guide arm comprising a guide head and a hand grip, the guide head comprising a second one or more protrusions, the second one or more protrusions extending towards the inferior direction,
at least a portion of the guide tool assembly removably and pivotally coupled to the handle allowing the first one or more protrusions of the keel rasp to be coaxially aligned to the second one or more protrusions of the guide tool assembly;
the guide tool assembly movable relative to the keel rasp from a first position, the first position comprising the keel rasp head is spaced apart at a first distance from the guide head, to a second position, the second position comprising the keel rasp head is spaced apart at a second distance, the second distance is greater than the first distance.

11. The keel alignment system of claim 10, wherein the handle comprises a powered handle or a manual handle.

12. The keel alignment system of claim 10, wherein keel rasp head is parallel relative to the guide head.

13. The keel alignment system of claim 10, wherein the first one or more protrusions of the keel rasp head of the keel rasp comprise a plurality of sharp teeth.

14. The keel alignment system of claim 10, wherein the second one or more protrusions comprise a plurality of flat and smooth surfaces.

15. The keel alignment system of claim 10, wherein the second one or more protrusions of the guide head of the movable guide assembly is sized and configured to be disposed into a keel channel.

16. An implant deployment system comprising:
an implant; and
a deployment assembly, the deployment assembly comprising a handle subassembly, a shaft subassembly, and a draw bar, the handle subassembly comprising a locking knob, the locking knob including a threaded bore and a channel,
the shaft subassembly comprising a grasping tip and a shaft, the shaft comprising a first end, a second end, and a first threads, the first threads disposed between a first end and a second end, the grasping tip coupled to the first end, the shaft being coupled to the locking knob by engaging the first threads with the threaded bore,
the draw bar comprising a first end and a second end, at least a portion of the draw bar being disposed over a portion of the shaft subassembly, a portion of the second end being disposed within the channel of the locking knob, at least a portion of the first end being disposed within the grasping tip of the shaft subassembly,
at least a portion of the implant being coupled to the grasping tip of the shaft subassembly, the shaft subassembly being movable from a first position having the implant locked in a planar orientation, to a second position having the implant unlocked by rotating the locking knob to push or slide the implant forward relative to the longitudinal axis of the deployment assembly.

17. The implant deployment system of claim 16, wherein the implant comprises a spinal implant.

18. The implant deployment system of claim 17, wherein the spinal implant comprises a superior component and inferior component, the superior component comprising a convex articulation element and the inferior component comprising a concave articulation element, the convex articulation element of the superior component contacts or engages the concave inferior articulation element to allow motion between the superior component and the inferior component.

19. The implant deployment system of claim 18, wherein the second end of the implant is coupled to a first end of the grasping tip of the actuating subassembly or at least a portion of the implant being coupled to the grasping tip of the shaft subassembly further comprises locking or immobilizing the superior component relative to the inferior component to prevent motion.

20. The implant deployment system of claim 18, wherein the second end of the implant is coupled to a first end of the grasping tip of the actuating subassembly or at least a portion of the implant being coupled to the grasping tip of the shaft subassembly further comprises locking or immobilizing the superior component relative to the inferior component to prevent motion.
